(19) United States
(12) Patent Application Publication
(10) Pub. No.: US 2024/0299376 A1
(43) Pub. Date: Sep. 12, 2024

(54) METHOD OF TREATING A CONDITION USING A THERAPEUTICALLY EFFECTIVE DOSE OF 1-(1-OXO-1,2-DIHYDROISOQUINOLIN-5-YL)-5-(TRIFLUOROMETHYL)-N-(2-(TRIFLUOROMETHYL)PYRIDIN-4-YL)-1H-PYRAZOLE-4-CARBOXAMIDE

(71) Applicant: Janssen Pharmaceutica NV, Beerse (BE)

(72) Inventors: Anthony T. GREWAY, Bound Brook, NJ (US); Ulrike PHILIPPAR, Antwerp (BE); Thomas C. WILDE, Philadelphia, PA (US); Marie E. WILLEMIN, Antwerpen (BE)

(21) Appl. No.: 18/279,556

(22) PCT Filed: Mar. 3, 2021
(86) PCT No.: PCT/IB2021/051776

§ 371 Date: Aug. 30, 2023

(51) Int. Cl. A61K 31/4725; A61P 35/00

(52) U.S. Cl. CPC A61K 31/4725; A61P 35/00

(57) ABSTRACT

The invention relates to a method of treating a disorder or condition that is affected by the inhibition of MALT1 in a subject in need of treatment, comprising administering a therapeutically effective dose of 1-(1-oxo-1,2-dihydroisoquinolin-5-yl)-5-(trifluoromethyl)-N-[2-(trifluoromethyl)pyridin-4-yl]-1H-pyrazole-4-carboxamide (Compound A):

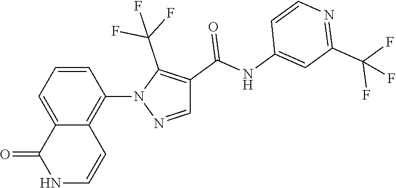 [SMILES: O=C(Nc1ccnc(C(F)(F)F)c1)c1cnn(-c2cccc3c(=O)[nH]ccc23)c1C(F)(F)F]

or a solvate or pharmaceutically acceptable salt form thereof to said subject, wherein said therapeutically effective dose is defined in the specification.

### FIELD OF THE INVENTION

The present invention relates to a method of treating a disease, syndrome, condition, or disorder in a subject, including a mammal and/or human in which the disease, syndrome, condition, or disorder is affected by the inhibition of MALT1, including but not limited to, cancer and/or immunological diseases, by administering such subject with a therapeutically effective dose of 1-(1-oxo-1,2-dihydroisoquinolin-5-yl)-5-(trifluoromethyl)-N-[2-(trifluoromethyl)pyridin-4-yl]-1H-pyrazole-4-carboxamide, or a solvate or pharmaceutically acceptable salt form thereof.

### BACKGROUND OF THE INVENTION

MALT1 (mucosa-associated lymphoid tissue lymphoma translocation 1) is a key mediator of the nuclear factor kappa-light-chain-enhancer of activated B cells (NF-κB) signaling pathway and has been shown to play a critical role in different types of lymphoma, including activated B cell-like (ABC) subtype of diffuse large B-cell lymphoma (DLBCL). MALT1 is the only human paracaspase that transduces signals from the B cell receptor (BCR) and T cell receptor (TCR). MALT1 is the active subunit of the CBM complex which is formed upon receptor activation. The “CBM complex” consists of multiple subunits of three proteins: CARD11 (caspase recruitment domain family member 11), BCL10 (B-cell CLL/Lymphoma 10) and MALT1.

MALT1 affects NF-κB signaling by two mechanisms: firstly, MALT1 functions as a scaffolding protein and recruits NF-κB signaling proteins such as TRAF6, TAB-TAK1 or NEMO-IKKα/β; and secondly, MALT1, as a cysteine protease, cleaves and thereby deactivates negative regulators of NF-κB signaling, such as RelB, A20 or CYLD. The ultimate endpoint of MALT1 activity is the nuclear translocation of the FKB transcription factor complex and activation of FKB signaling (Jaworski et al., Cell Mol Life Science 2016. 73, 459-473).

Non-Hodgkin lymphoma represents a diverse set of diseases, of which more than 60 subtypes have been identified (https://www.cancer.net/cancer-types/lymphoma-non-hodgkin/subtypes). Worldwide, DLBCL represents the most common subtype of NHL, accounting for 30% to 40% of all newly diagnosed cases (Sehn L H, Gascoyne R D. Diffuse large B-cell lymphoma: optimizing outcome in the context of clinical and biologic heterogeneity. Blood. 2015; 125(1):22-32). DLBCL typically presents as an aggressive lymphoma, evolving over months and resulting in symptomatic disease that is fatal without treatment (Ibid).

Constitutive activation of NF-κB signaling is the hallmark of ABC-DLBCL (Diffuse Large B Cell Lymphoma of the Activated B Cell-like subtype), the more aggressive form of DLBCL. DLBCL is the most common form of non-Hodgkin's lymphoma (NHL), accounting for approximately 25% of lymphoma cases while ABC-DLBCL comprises approximately 40% of DLBCL. NF-κB pathway activation is driven by mutations of signaling components, such as CD79A/B, CARD11, MYD88 or A20, in ABC-DLBCL patients (Staudt, Cold Spring Harb Perspect Biol 2010 June; 2(6); Lim et al., Immunol Rev 2012, 246, 359-378).

Outcomes in DLBCL have improved dramatically over the last decade with the addition of rituximab to cyclophosphamide, doxorubicin, vincristine, and prednisone (R CHOP). This regimen remains the current standard of care. However, R CHOP treatment fails in about 30% to 50% of patients with DLBCL (Coiffier B, Sarkozy C. Diffuse large B-cell lymphoma: R-CHOP failure-what to do? Hematology Am Soc Hematol Educ Program. 2016; 2016(1):366-378). Less than half of these patients can be cured with stem cell transplantation (Gisselbrecht C, Glass B, Mounier N, et al. Salvage regimens with autologous transplantation for relapsed large B-cell lymphoma in the rituximab era. J Clin Oncol. 2010; 28(27): 4184-4190), and those who are not cured will typically die from their disease (Crump M, Neelapu S S, Farooq U, et al. Outcomes in refractory diffuse large B-cell lymphoma: results from the international SCHOLAR-1 study. Blood. 2017; 130(16):1800-1808). Since the best chance for cure is front-line treatment, there have been many attempts to improve upon R CHOP but so far, these treatments have failed to significantly improve outcomes (Goy A. Succeeding in breaking the R-CHOP ceiling in DLBCL: Learning from negative trials. J Clin Oncol. 2017; 35(31): 3519-3522). Recently, several studies have explored the addition of targeted agents to R CHOP in front-line treatment. Promising signs of activity in some of these studies encourage the further exploration of combinations that may improve cure rate of targeted agents in select patients (Chiappella A, Witzig T E, Vitolo U, et al. ROBUST: Phase III randomized study of lenalidomide/R-CHOP vs placebo/R-CHOP in untreated ABC-type diffuse large B-cell lymphoma and feasibility of cell of origin subtyping. Hematological Oncology. 2017; 35(S2):419-428, Younes A, Thieblemont C, Morschhauser F, et al. Combination of ibrutinib with rituximab, cyclophosphamide, doxorubicin, vincristine, and prednisone (R-CHOP) for treatment-naive patients with CD20-positive B-cell non-Hodgkin lymphoma: a non-randomised, phase 1b study. Lancet Oncol. 2014; 15(9): 1019-1026). Thus, optimization of front-line therapy, as well as the development of more effective salvage strategies, remains an important objective.

Follicular lymphoma (FL), mucosa-associated lymphoid tissue (MALT) lymphoma, chronic lymphocytic leukemia (CLL), small lymphocytic lymphoma (SLL), mantle cell lymphoma (MCL), and Waldenström macroglobulinemia (WM) are considered largely incurable lymphomas that require therapies throughout the course of disease. Currently, there are limited lines of therapy available for these diseases, and treatments are needed that avoid the use of cytotoxic chemotherapy.

The use of BTK inhibitors, for example Ibrutinib, provides clinical proof-of-concept that inhibiting NF-κB signaling in ABC-DLBCL is efficacious. MALT1 is downstream of BTK in the NF-κB signaling pathway and a MALT1 inhibitor could target ABC-DLBCL patients not responding to Ibrutinib, mainly patients with CARD11 mutations, as well as treat patients that acquired resistance to Ibrutinib.

Small molecule tool compound inhibitors of MALT1 protease have demonstrated efficacy in preclinical models of ABC-DLBCL (Fontan et al., Cancer Cell 2012, 22, 812-824; Nagel et al., Cancer Cell 2012, 22, 825-837). Interestingly, covalent catalytic site and allosteric inhibitors of MALT1 protease function have been described, suggesting that inhibitors of this protease may be useful as pharmaceutical agents (Demeyer et al., Trends Mol Med 2016, 22, 135-150).

The chromosomal translocation creating the API2-MALT1 fusion oncoprotein is the most common mutation identified in MALT (mucosa-associated lymphoid tissue) lymphoma. API2-MALT1 is a potent activator of the NF-κB pathway (Rosebeck et al., World J Biol Chem 2016, 7, 128-137). API2-MALT1 mimics ligand-bound TNF receptor and promotes TRAF2-dependent ubiquitination of RIP1 which acts as a scaffold for activating canonical NF-κB signaling. Furthermore, API2-MALT1 has been shown to cleave and generate a stable, constitutively active fragment of NF-κB-inducing kinase (NIK) thereby activating the non-canonical NF-κB pathway (Rosebeck et al., Science, 2011, 331, 468-472).

In addition to lymphomas, MALT1 has been shown to play a critical role in innate and adaptive immunity (Jaworski M, et al., Cell Mol Life Sci. 2016). MALT1 protease inhibitor can attenuate disease onset and progression of mouse experimental allergic encephalomyelitis, a mouse model of multiple sclerosis (Mc Guire et al., J. Neuroinflammation 2014, 11, 124). Mice expressing catalytically inactive MALT1 mutant showed loss of marginal zone B cells and B1B cells and general immune deficiency characterized as decreased T and B cell activation and proliferation. However, those mice also developed spontaneous multi-organ autoimmune inflammation at the age of 9 to 10 weeks. It is still poorly understood why MALT1 protease dead knock-in mice show a break of tolerance while conventional MALT1 KO mice do not. One hypothesis suggests the unbalanced immune homeostasis in MALT1 protease dead knock-in mice may be caused by incomplete deficiency in T and B cell but severe deficiency of immunoregulatory cells (Jaworski et al., EMBO J. 2014; Gewies et al., Cell Reports 2014; Bornancin et al., J. Immunology 2015; Yu et al., PLOS One 2015). Similarly, MALT deficiency in humans has been associated with combined immunodeficiency disorder (Mckinnon et al., J. Allergy Clin. Immunol. 2014, 133, 1458-1462; Jabara et al., J. Allergy Clin. Immunol. 2013, 132, 151-158; Punwani et al., J. Clin. Immunol. 2015, 35, 135-146). Given the difference between genetic mutation and pharmacological inhibition, a phenotype of MALT1 protease dead knock-in mice might not resemble that of patients treated with MALT1 protease inhibitors. A reduction of immunosuppressive T cells by MALT1 protease inhibition may be beneficial to cancer patients by potentially increasing antitumor immunity.

Thus, MALT1 inhibitors may provide a therapeutic benefit to patients suffering from cancer and/or immunological diseases. MALT1 inhibition can be effective in the treatment of ABC DLBCL and other DLBCL subtypes, MALT lymphoma, as well as CLL, MCL, and WM tumors, including tumors that are resistant to a Bruton tyrosine kinase inhibitor (BTKi).

### SUMMARY OF THE INVENTION

The present invention relates to methods of treating a disorder or condition that is affected by the inhibition of MALT1 in a subject in need of treatment, comprising administering a therapeutically effective dose ranging from about 50 to about 1000 mg, alternatively from about 100 to about 1000 mg, alternatively about 300 mg of 1-(1-oxo-1,2-dihydroisoquinolin-5-yl)-5-(trifluoromethyl)-N-[2-(trifluoromethyl)pyridin-4-yl]-1H-pyrazole-4-carboxamide (Compound A):

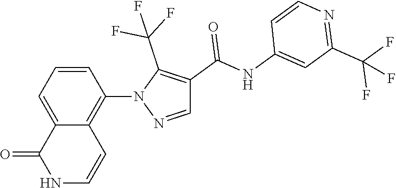 [SMILES: O=C(Nc1ccnc(C(F)(F)F)c1)c1cnn(-c2cccc3c(=O)[nH]ccc23)c1C(F)(F)F]

or an enantiomer, diastereomer, a solvate or a pharmaceutically acceptable salt form thereof to said subject.

Additionally, the invention relates to methods of reducing the T_{reg}/Teff ratio in a patient suffering from a disorder or condition that is affected by the inhibition of MALT1 comprising administering a therapeutically effective dose of 1-(1-oxo-1,2-dihydroisoquinolin-5-yl)-5-(trifluoromethyl)-N-[2-(trifluoromethyl)pyridin-4-yl]-1H-pyrazole-4-carboxamide (Compound A):

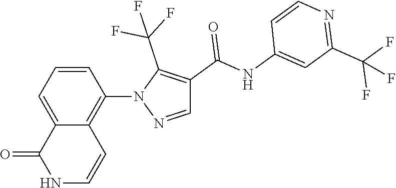 [SMILES: O=C(Nc1ccnc(C(F)(F)F)c1)c1cnn(-c2cccc3c(=O)[nH]ccc23)c1C(F)(F)F]

or an enantiomer, diastereomer, a solvate or a pharmaceutically acceptable salt form thereof to said patient.

The present invention also relates to a therapeutically effective dose ranging from about 25 to about 1000 mg, alternatively from about 50 to about 1000 mg, alternatively from about 100 to about 1000 mg of Compound A or pharmaceutically acceptable salt form thereof for use in treating a disorder or condition that is affected by the inhibition of MALT1. In addition, the present invention relates to use of a therapeutically effective dose ranging from about 50 to about 1000 mg, alternatively from about 100 to about 1000 mg of Compound A or pharmaceutically acceptable salt form thereof for treating a disorder or condition that is affected by the inhibition of MALT1. Additionally, the present invention relates to use of a therapeutically effective dose ranging from about 50 to about 1000 mg, alternatively from about 100 to about 1000 mg of Compound A or pharmaceutically acceptable salt form thereof in the manufacture of a medicament for treating a disorder or condition that is affected by the inhibition of MALT1.

In one embodiment, the disorder or condition is cancer and/or immunological disease. In another embodiment, the disorder or condition is lymphoma, such as, for example chronic lymphocytic leukemia (CLL) or small lymphocytic lymphoma (SLL). In yet another embodiment, disorder or condition is selected from the group consisting of diffuse large B-cell lymphoma (DLBCL), mantle cell lymphoma (MCL), follicular lymphoma (FL), and mucosa-associated lymphoid tissue (MALT) lymphoma. In another embodiment, the disorder or condition is the activated B cell like (ABC) subtype of diffuse large B-cell lymphoma (DLBCL).

### BRIEF DESCRIPTION OF THE DRAWINGS

The file of this patent or application contains at least one drawing/photograph executed in color. All drawings/photographs in colour also have corresponding versions in black and white. Copies of this patent or patent application publication with color drawing(s)/photograph(s) will be provided by the Office upon request and payment of the necessary fee.

The summary, as well as the following detailed description, is further understood when read in conjunction with the appended drawings. For the purpose of illustrating the invention, there are shown in the drawings exemplary embodiments of the invention; however, the invention is not limited to the specific disclosure of the drawings. In the drawings:

FIG. 1 is a plot of serum IL-10 and PK exposure after a single dose of Compound A in OCI-LY3 tumor-bearing male NSG mice.

FIG. 2 is a plot of uncleaved BCL10 after a single dose of Compound A in OCI-LY3 tumor-bearing male NSG mice.

FIG. 3 is a plot of serum IL-10 and PK exposure after a single dose of Compound A in OCI-LY10 tumor-bearing female NSG mice.

FIG. 4 is a plot of the effect of Compound A on the growth of established OCI-LY3 Human DLBCL xenografts in male NSC mice.

FIG. 5 is a plot of the effect of Compound A on the growth of established OCI-LY10 Human DLBCL xenografts in female NSC mice.

FIG. 6 is a Western blot showing RelB cleavage in OCI-LY3 cells upon treatment with Compound A.

FIG. 7 is a plot showing uncleaved BCL10 in OCI-LY3 cells upon treatment with Compound A.

FIG. 8 is a plot showing RelB cleavage in BJAB cells overexpressing API2-MALT1 upon treatment with Compound A.

FIG. 9 shows a Western Blot assessing MALT1 scaffolding function in OCI-LY3 cells upon treatment with Compound A.

FIG. 10 is a plot showing the anti-proliferative activity of Compound A in a NHL cell line panel.

FIG. 11A and FIG. 11B are plots showing the anti-proliferative activity of ibrutinib (FIG. 11A) or Compound A (FIG. 11B) in TMD8 cells and TMD8 cell lines engineered to mimic ibrutinib resistance.

FIG. 12 is a plot showing the fluorescence-activated cell sorting (FACS) analysis upon treatment with Compound A. In FIG. 12, NOSTIM means no stimulation; STIM means CD3/28 stimulation. The following T_{reg} population was used for the analysis: CD4⁺, CD25⁺, FoxP3^{hi}. FIG. 12 is in color and FIG. 21 is a corresponding black and white version.

FIG. 13 is a plot showing the T_{reg}/T_{eff} ratio upon treatment with Compound A.

FIG. 14 is a plot showing cytometry by time of flight (“CyTOF”) of T-cell populations identified in SPADE tree and MALT1 expression measured by CyTOF in T cells upon treatment with Compound A. FIG. 14 is in color and FIG. 22 is a corresponding black and white version.

FIG. 15A is a plot showing the T_{reg}/T_{eff} ratio upon treatment with Compound A as measured by CyTOF.

FIG. 15B is a plot showing the CD8+ population upon treatment with Compound A as measured by CyTOF.

FIG. 16A is a plot showing expression of exhaustion marker PD-1 on CD8⁺ T cells upon treatment with Compound A as measured by CyTOF.

FIG. 16B is a plot showing expression of exhaustion marker LAG3 on CD8⁺ T cells upon treatment with Compound A as measured by CyTOF.

FIG. 16C is a plot showing expression of exhaustion marker CTLA4 on CD8⁺ T cells upon treatment with Compound A as measured by CyTOF.

FIG. 17 shows Radviz plots generated from CyTOF panel analyzing multiple markers after CD3/28 simulation with or without Compound A treatment. FIG. 17 is in color and FIG. 23 is a corresponding black and white version.

FIG. 18 is a plot showing plasma concentrations (in ng/ml) following administration of different formulations of Compound A in dogs. PO, oral gavage; PEG400 solution for oral, PEG400/water 70:30 for IV; susp, 0.5% HPMC suspension.

FIG. 19A is a plot showing the plasma concentrations (in ng/ml) following administration of multiple doses of Compound A in male rats.

FIG. 19B is a plot showing the plasma concentrations (in ng/ml) following administration of multiple doses of Compound A in female rats.

FIG. 20 is a plot showing the plasma concentrations (in ng/ml) following administration of multiple doses of Compound A in female dogs.

FIG. 21 is a plot showing the fluorescence-activated cell sorting (FACS) analysis upon treatment with Compound A. In FIG. 21, NOSTIM means no stimulation; STIM means CD3/28 stimulation. The following T_{reg} population was used for the analysis: CD4⁺, CD25⁺, FoxP3^{hi}. FIG. 21 is a black and white version of FIG. 12.

FIG. 22 is a plot showing cytometry by time of flight (“CyTOF”) of T-cell populations identified in SPADE tree and MALT1 expression measured by CyTOF in T cells upon treatment with Compound A. FIG. 22 is a black and white version of FIG. 14.

FIG. 23 shows Radviz plots generated from CyTOF panel analysing multiple markers after CD3/28 simulation with or without Compound A treatment. FIG. 23 is a black and white version of FIG. 17.

### DETAILED DESCRIPTION OF THE INVENTION

Various publications, articles and patents are cited or described in the background and throughout the specification; each of these references is herein incorporated by reference in its entirety. Discussion of documents, acts, materials, devices, articles or the like which has been included in the present specification is for the purpose of providing context for the invention. Such discussion is not an admission that any or all of these matters form part of the prior art with respect to any inventions disclosed or claimed.

All patents, published patent applications, and publications cited herein are incorporated by reference as if set forth fully herein.

Unless defined otherwise, all technical and scientific terms used herein have the same meaning commonly understood to one of ordinary skill in the art to which this invention pertains. Otherwise, certain terms used herein have the meanings as set in the specification.

### Definitions

Some of the quantitative expressions given herein are not qualified with the term “about”. It is understood that whether the term “about” is used explicitly or not, every quantity given herein is meant to refer to the actual given value, and it is also meant to refer to the approximation to such given value that would reasonably be inferred based on the ordinary skill in the art, including approximations due to the experimental and/or measurement conditions for such given value.

Throughout the description and claims of this specification, the words “comprise” and “contain” and variations of the words, for example “comprising” and “comprises”, mean “including but not limited to”, and are not intended to (and do not) exclude other components. As used herein and in the appended claims, the singular forms “a,” “an,” and “the” include plural reference unless the context clearly dictates otherwise.

The term “Compound A” refers to 1-(1-oxo-1,2-dihydroisoquinolin-5-yl)-5-(trifluoromethyl)-N-[2-(trifluoromethyl)pyridin-4-yl]-1H-pyrazole-4-carboxamide having the following structure:

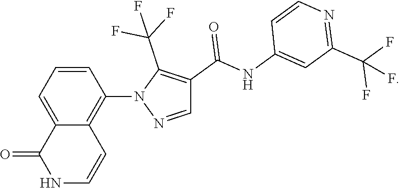 [SMILES: O=C(Nc1ccnc(C(F)(F)F)c1)c1cnn(-c2cccc3c(=O)[nH]ccc23)c1C(F)(F)F]

The invention also contemplates Compound A or an enantiomer, diastereomer, a solvate or a pharmaceutically acceptable salt thereof, and considers them to be within the scope of the invention.

Compound A may be prepared, for example, as described in Example 158 of WO 2018/119036 and WO 2020/169736, which are incorporated herein by reference. The procedure of Example 158 has been determined as providing Compound A hydrate.

Compound A may exist as a solvate. A “solvate” may be a solvate with water (i.e., a hydrate) or with a common organic solvent. The use of pharmaceutically acceptable solvates, said solvates including hydrates, and said hydrates including mono-hydrates, is considered to be within the scope of the invention.

Compound A may be formulated in an amorphous form or dissolved state; for example, and without limitation, Compound A may be formulated in an amorphous form with a polyethylene glycol (PEG) polymer.

A person of ordinary skill in the art would recognize that Compound A may exist as tautomers. It is understood that all tautomeric forms are encompassed by a structure where one possible tautomeric arrangement of the groups of the compound is described, even if not specifically indicated.

For example, it is understood that:

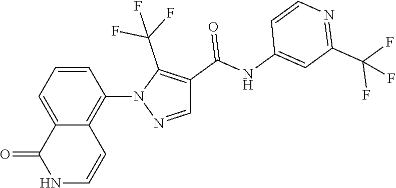 [SMILES: O=C(Nc1ccnc(C(F)(F)F)c1)c1cnn(-c2cccc3c(=O)[nH]ccc23)c1C(F)(F)F]

also encompasses by the following structure:

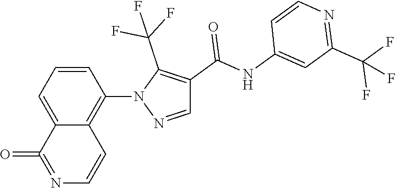

Any convenient tautomeric arrangement may be utilized in describing the compounds.

For use in medicine, salts of Compound A refer to non-toxic “pharmaceutically acceptable salts.”

“Pharmaceutically acceptable” means approved or approvable by a regulatory agency of the Federal or a state government or the corresponding agency in countries other than the United States, or that is listed in the U.S. Pharmacopoeia or other generally recognized pharmacopoeia for use in animals, and more particularly, in humans. Suitable pharmaceutically acceptable salts of Compound A include acid addition salts that can, for example, be formed by mixing a solution of the compound with a solution of a pharmaceutically acceptable acid such as, hydrochloric acid, sulfuric acid, fumaric acid, maleic acid, succinic acid, acetic acid, benzoic acid, citric acid, tartaric acid, carbonic acid, or phosphoric acid. Furthermore, where the compounds carry an acidic moiety, suitable pharmaceutically acceptable salts thereof may include alkali metal salts such as, sodium or potassium salts; alkaline earth metal salts such as, calcium or magnesium salts; and salts formed with suitable organic ligands such as, quaternary ammonium salts. Thus, representative pharmaceutically acceptable salts include acetate, benzenesulfonate, benzoate, bicarbonate, bisulfate, bitartrate, borate, bromide, calcium edetate, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isothionate, lactate, lactobionate, laurate, malate, maleate, mandelate, mesylate, methylbromide, methylnitrate, methylsulfate, mucate, napsylate, nitrate, N-methylglucamine ammonium salt, oleate, pamoate (embonate), palmitate, pantothenate, phosphate/diphosphate, polygalacturonate, salicylate, stearate, sulfate, subacetate, succinate, tannate, tartrate, teoclate, tosylate, triethiodide, and valerate.

The term “subject” means any animal, particularly a mammal, most particularly a human, who will be or has been treated by a method according to an embodiment of the invention. The term “mammal” as used herein, encompasses any mammal. Examples of mammals include, but are not limited to, cows, horses, sheep, pigs, cats, dogs, mice, rats, rabbits, guinea pigs, non-human primates (NHPs) such as monkeys or apes, humans, etc., more particularly a human.

The term “therapeutically effective dose” refers to an amount of an active compound or pharmaceutical agent, including a crystalline form of the present invention, which elicits the biological or medicinal response in a tissue system, animal or human that is being sought by a researcher, veterinarian, medical doctor or other clinician, including reduction or inhibition of an enzyme or a protein activity, or ameliorating symptoms, alleviating conditions, slowing or delaying disease progression, or preventing a disease.

Where doses of the present invention are expressed in relation to the weight of the subject, “mg/kg” is used to specify milligrams of the compound for each kilogram of the subject's body weight.

In one embodiment, the term “therapeutically effective dose” refers to the amount of Compound A enantiomer, diastereomer, solvate or an or a pharmaceutically acceptable salt form thereof, that when administered to a subject, is effective to (1) at least partially alleviate, inhibit, prevent, and/or ameliorate a condition, or a disorder or a disease (i) mediated by MALT1; or (ii) associated with MALT1 activity; or (iii) characterized by activity (normal or abnormal) of MALT1; or (2) reduce or inhibit the activity of MALT1; or (3) reduce or inhibit the expression of MALT1; or (4) modify the protein levels of MALT1.

The term “composition” refers to a product that includes the specified ingredients in therapeutically effective amounts, as well as any product that results, directly, or indirectly, from combinations of the specified ingredients in the specified amounts.

The term “administer” or “administered” or “administering” refers to the administration of Compound A or a solvate or pharmaceutically acceptable salt form thereof, or a pharmaceutical composition thereof to a subject by any method known to those skilled in the art in view of the present disclosure, such as by intramuscular, subcutaneous, oral, intravenous, cutaneous, intramucosal (e.g., gut), intranasal or intraperitoneal route of administration. In particular embodiments, a pharmaceutical composition of the invention is administered to a subject orally.

The term “affected by the inhibition of MALT1” in the context of a disorder or disease refers to any disease, syndrome, condition, or disorder that might occur in the absence of MALT1 but can occur in the presence of MALT1. Suitable examples of a disease, syndrome, condition, or disorder that is affected by the inhibition of MALT1 include, but are not limited to, lymphomas, leukemias, carcinomas, and sarcomas, e.g. non-Hodgkin's lymphoma (NHL (including B-cell NHL)), diffuse large B-cell lymphoma (DLBCL), mantle cell lymphoma (MCL), follicular lymphoma (FL), mucosa-associated lymphoid tissue (MALT) lymphoma, marginal zone lymphoma, T-cell lymphoma, Hodgkin's lymphoma, Burkitt's lymphoma, multiple myeloma, chronic lymphocytic leukemia (CLL), small lymphocytic lymphoma (SLL), Waldenström macroglobulinemia, lymphoblastic T cell leukemia, chronic myelogenous leukemia (CML), hairy-cell leukemia, acute lymphoblastic T cell leukemia, plasmacytoma, immunoblastic large cell leukemia, megakaryoblastic leukemia, acute megakaryocytic leukemia, promyelocytic leukemia, erythroleukemia, brain (gliomas), glioblastomas, breast cancer, colorectal/colon cancer, prostate cancer, lung cancer including non-small-cell, gastric cancer, endometrial cancer, melanoma, pancreatic cancer, liver cancer, kidney cancer, squamous cell carcinoma, ovarian cancer, sarcoma, osteosarcoma, thyroid cancer, bladder cancer, head and neck cancer, testicular cancer, Ewing's sarcoma, rhabdomyosarcoma, medulloblastoma, neuroblastoma, cervical cancer, renal cancer, urothelial cancer, vulval cancer, esophageal cancer, salivary gland cancer, nasopharyngeal cancer, buccal cancer, cancer of the mouth, primary and secondary central nervous system lymphoma, transformed follicular lymphoma, diseases/cancer caused by API2-MALT1 fusion, and GIST (gastrointestinal stromal tumor). Additional examples include, but is not limited to, autoimmune and inflammatory disorders, e.g. arthritis, inflammatory bowel disease, gastritis, ankylosing spondylitis, ulcerative colitis, pancreatitis, Crohn's disease, celiac disease, multiple sclerosis, systemic lupus erythematosus, lupus nephritis, rheumatic fever, gout, organ or transplant rejection, chronic allograft rejection, acute or chronic graft-versus-host disease, dermatitis including atopic, dermatomyositis, psoriasis, Behcet's diseases, uveitis, myasthenia gravis, Grave's disease, Hashimoto thyroiditis, Sjoergen's syndrome, blistering disorders, antibody-mediated vasculitis syndromes, immune-complex vasculitides, allergic disorders, asthma, bronchitis, chronic obstructive pulmonary disease (COPD), cystic fibrosis, pneumonia, pulmonary diseases including oedema, embolism, fibrosis, sarcoidosis, hypertension and emphysema, silicosis, respiratory failure, acute respiratory distress syndrome, BENTA disease, berylliosis, and polymyositis.

As used herein, the term “condition” refers to any disease, syndrome, or disorder detected or diagnosed by a researcher, veterinarian, medical doctor, or other clinician, wherein said researcher, veterinarian, medical doctor, or other clinician determines that it desirable to seek a biological or medicinal response in an animal tissue system, particularly a mammalian or human tissue system.

As used herein, the term “disorder” refers to any disease, syndrome, or condition detected or diagnosed by a researcher, veterinarian, medical doctor, or other clinician, wherein said researcher, veterinarian, medical doctor, or other clinician determines that it desirable to seek a biological or medicinal response in an animal tissue system, particularly a mammalian or human tissue system.

As used herein, the term “MALT1 inhibitor” refers to an agent that inhibits or reduces at least one condition, symptom, disorder, and/or disease of MALT1.

As used herein, unless otherwise noted, the term “affect” or “affected” (when referring to a disease, syndrome, condition or disorder that is affected by the inhibition of MALT1) includes a reduction in the frequency and/or severity of one or more symptoms or manifestations of said disease, syndrome, condition or disorder; and/or includes the prevention of the development of one or more symptoms or manifestations of said disease, syndrome, condition or disorder or the development of the disease, condition, syndrome or disorder.

As used herein, the term “treat”, “treating”, or “treatment” of any disease, condition, syndrome, or disorder refers, in one embodiment, to ameliorating the disease, condition, syndrome or disorder (i.e. slowing or arresting or reducing the development of the disease or at least one of the clinical symptoms thereof). In another embodiment, “treat”, “treating”, or “treatment” refers to alleviating or ameliorating at least one physical parameter including those which may not be discernible by the patient. In a further embodiment, “treat”, “treating”, or “treatment” refers to modulating the disease, condition, syndrome, or disorder either physically (e.g. stabilization of a discernible symptom), physiologically, (e.g. stabilization of a physical parameter), or both. In yet another embodiment, “treat”, “treating”, or “treatment” refers to preventing or delaying the onset or development or progression of the disease, condition, syndrome, or disorder.

A skilled person will understand that references to Compound A might also refer to an enantiomer, diastereomer, a solvate or a pharmaceutically acceptable salt form thereof, even if not explicitly referred to, and that they are also included in the scope of the present invention.

### Embodiments

### Compositions

Even though the compounds of embodiments of the present invention can be administered alone, they will generally be administered in admixture with a pharmaceutically acceptable carrier, a pharmaceutically acceptable excipient and/or a pharmaceutically acceptable diluent selected with regard to the intended route of administration and standard pharmaceutical or veterinary practice. Thus, particular embodiments of the present invention are directed to pharmaceutical and veterinary compositions comprising Compound A and at least one pharmaceutically acceptable carrier, pharmaceutically acceptable excipient, and/or pharmaceutically acceptable diluent.

By way of example, in the pharmaceutical compositions of embodiments of the present invention, Compound A may be admixed with any suitable binder(s), lubricant(s), suspending agent(s), coating agent(s), solubilizing agent(s), and combinations thereof.

Solid oral dosage forms such as, tablets or capsules, containing Compound A may be administered in at least one dosage form at a time, as appropriate. It is also possible to administer Compound A in sustained release formulations. Alternatively, Compound A may be administered as a sprinkle formulation.

Additional oral forms in which Compound A may be administered include elixirs, solutions, syrups, and suspensions; each optionally containing flavoring agents and coloring agents.

Alternatively, Compound A may be administered by inhalation (intratracheal or intranasal) or in the form of a suppository or pessary, or they may be applied topically in the form of a lotion, solution, cream, ointment, or dusting powder. For example, compounds can be incorporated into a cream comprising, consisting of, and/or consisting essentially of an aqueous emulsion of polyethylene glycols or liquid paraffin. They can also be incorporated, at a concentration of between about 1% and about 10% by weight of the cream, into an ointment comprising, consisting of, and/or consisting essentially of a wax or soft paraffin base together with any stabilizers and preservatives as may be required. An alternative means of administration includes transdermal administration using a skin or transdermal patch.

The pharmaceutical compositions of the present invention (as well as the compounds of the present invention alone) can also be injected parenterally, for example, intracavernosally, intravenously, intramuscularly, subcutaneously, intradermally, or intrathecally. In this case, the compositions will also include at least one of a suitable carrier, a suitable excipient, and/or a suitable diluent.

For parenteral administration, the pharmaceutical compositions of the present invention are best used in the form of a sterile aqueous solution that may contain other substances, for example, enough salts and monosaccharides to make the solution isotonic with blood.

For oral, buccal or sublingual administration, the pharmaceutical compositions of the present invention may be administered in the form of tablets, gelatin capsules, or lozenges, which can be formulated in a conventional manner.

By way of further example, pharmaceutical compositions containing Compound A as the active pharmaceutical ingredient can be prepared by mixing Compound A with a pharmaceutically acceptable carrier, a pharmaceutically acceptable diluent, and/or a pharmaceutically acceptable excipient according to conventional pharmaceutical compounding techniques. The carrier, excipient, and diluent may take a wide variety of forms depending upon the desired route of administration (e.g., oral, parenteral, intramuscular, subcutaneous, intravenous, cutaneous, intramucosal (e.g., gut), intranasal or intraperitoneal routes). Thus, for liquid oral preparations such as, suspensions, syrups, elixirs and solutions, suitable carriers, excipients and diluents include water, glycols, oils, alcohols, flavoring agents, preservatives, stabilizers, coloring agents and the like; for solid oral preparations such as, powders, capsules, and tablets, suitable carriers, excipients and diluents include starches, sugars, diluents, granulating agents, lubricants, binders, disintegrating agents, stabilizers (eg., copovidone), solubilizers (eg, PEG 400, PEG 1500), antioxidants (eg, alpha-tocopherol) and the like. Solid oral preparations also may be optionally coated with substances such as, sugars, or be enterically coated so as to modulate the major site of absorption and disintegration. For parenteral administration, the carrier, excipient, and diluent will usually include sterile water, and other ingredients may be added to increase solubility and preservation of the composition. Injectable suspensions or solutions may also be prepared utilizing aqueous carriers along with appropriate additives such as, solubilizers and preservatives.

For oral administration, a pharmaceutical composition containing Compound A may be provided in the form of tablets or gelatin capsules containing a therapeutically effective dose as disclosed below. In some embodiments, the tablet may be made from spray dried powder (SDP), wherein the SDP is a solid dispersion of Compound A in hypromellose acetate succinate (AS) (hydroxypropyl methylcellulose acetate succinate [HPMC]) polymer, in a 1:2 ratio. The tablet may contain about 100 mg of Compound A, alternatively about 150 mg of Compound A, alternatively about 200 mg of Compound A, alternatively about 250 mg of Compound A, alternatively about 300 mg of Compound A, alternatively about 350 mg of Compound A.

### Methods of Treatment

A skilled person will understand that references to Compound A in the section ‘Methods of Treatment’, might also refer to an enantiomer, diastereomer, a solvate or a pharmaceutically acceptable salt form thereof, even if not explicitly referred to, and that they are also included in the scope of the present invention.

One aspect of the invention is directed to methods of treating a disorder or condition that is affected by the inhibition of MALT1 in a subject in need of treatment, comprising administering a therapeutically effective dose of Compound A or a pharmaceutically acceptable salt form thereof. In some embodiments, the invention is directed to methods of treating a cancer or an immunological disease disclosed herein in a subject in need of treatment, comprising administering a therapeutically effective dose of Compound A or a pharmaceutically acceptable salt form thereof.

As noted, Compound A or pharmaceutically acceptable salt form thereof may be used for treating a disorder or condition that is affected by the inhibition of MALT1. In certain embodiments of the invention, the disorder or condition is cancer and/or an immunological disease. Accordingly, in one embodiment, the disorder or condition is cancer. Alternatively, in another embodiment, the disorder or condition is an immunological disease.

In yet another embodiment, the disorder or condition includes, but is not limited to cancers, such as lymphomas, leukemias, carcinomas, and sarcomas, e.g. non-Hodgkin's lymphoma (NHL (including B-cell NHL)), diffuse large B-cell lymphoma (DLBCL), mantle cell lymphoma (MCL), follicular lymphoma (FL), mucosa-associated lymphoid tissue (MALT) lymphoma, marginal zone lymphoma, T-cell lymphoma, Hodgkin's lymphoma, Burkitt's lymphoma, multiple myeloma, chronic lymphocytic leukemia (CLL), small lymphocytic lymphoma (SLL), Waldenström macroglobulinemia, lymphoblastic T cell leukemia, chronic myelogenous leukemia (CML), hairy-cell leukemia, acute lymphoblastic T cell leukemia, plasmacytoma, immunoblastic large cell leukemia, megakaryoblastic leukemia, acute megakaryocyte leukemia, promyelocytic leukemia, erythroleukemia, brain (gliomas), glioblastomas, breast cancer, colorectal/colon cancer, prostate cancer, lung cancer including non-small-cell, gastric cancer, endometrial cancer, melanoma, pancreatic cancer, liver cancer, kidney cancer, squamous cell carcinoma, ovarian cancer, sarcoma, osteosarcoma, thyroid cancer, bladder cancer, head & neck cancer, testicular cancer, Ewing's sarcoma, rhabdomyosarcoma, medulloblastoma, neuroblastoma, cervical cancer, renal cancer, urothelial cancer, vulval cancer, esophageal cancer, salivary gland cancer, nasopharyngeal cancer, buccal cancer, cancer of the mouth, primary and secondary central nervous system lymphoma, transformed follicular lymphoma, diseases/cancer caused by API2-MALT1 fusion, and GIST (gastrointestinal stromal tumor).

In an alternate embodiment, the disorder or condition is selected from non-Hodgkin's lymphoma (NHL), diffuse large B-cell lymphoma (DLBCL), marginal zone lymphoma, mantle cell lymphoma (MCL), follicular lymphoma (FL), transformed follicular lymphoma, chronic lymphocytic leukemia, and Waldenström macroglobulinemia.

In yet another embodiment of the invention, the disorder or condition is lymphoma. In another embodiment of the invention, the disorder or condition is the activated B cell like (ABC) subtype of diffuse large B-cell lymphoma (DLBCL). In another embodiment of the invention, the disorder or condition is germinal center B cell like (GCB) subtype of diffuse large B-cell lymphoma (DLBCL). In another embodiment of the invention, the disorder or condition is non-germinal center B cell like (non-GCB) subtype of diffuse large B-cell lymphoma (DLBCL).

In an additional embodiment of the invention, the disorder or condition is chronic lymphocytic leukemia (CLL). In another embodiment, the disorder or condition small lymphocytic lymphoma (SLL).

In another embodiment of the invention, the lymphoma is MALT lymphoma.

In another embodiment of the invention, the disorder or condition is Waldenström macroglobulinemia (WM).

In yet another embodiment, the disorder or condition is selected from the group consisting of diffuse large B-cell lymphoma (DLBCL), mantle cell lymphoma (MCL), follicular lymphoma (FL), and mucosa-associated lymphoid tissue (MALT) lymphoma.

In an alternate embodiment, the disorder or condition is non-Hodgkin's lymphoma (NHL). In a further embodiment, the non-Hodgkin's lymphoma (NHL) is B-cell NHL.

In yet another embodiment, the disorder or condition is primary and secondary central nervous system lymphoma, transformed follicular lymphoma, or API2-MALT1 fusion dependent disease.

In another embodiment of the invention, the disorder or condition (cancer or immunological disease (such as any of the cancers listed above)) is relapsed or refractory to prior treatment.

In another embodiment of the invention, the disorder or condition is cancer (such as any of the cancers mentioned above) and the subject has received prior treatment with a Bruton tyrosine kinase inhibitor (BTKi).

In an alternate embodiment of the invention, the disorder or condition is cancer (such as any of the cancers mentioned above) and the subject is relapsed or refractory to prior treatment with a Bruton tyrosine kinase inhibitor (BTKi).

In other embodiments, the disorder or condition is an immunological disease. Accordingly, in an embodiment, the disorder or condition is an immunological disease, syndrome, disorder, or condition selected from the group consisting of autoimmune and inflammatory disorders, e.g. arthritis, rheumatoid arthritis (RA), psoriatic arthritis (PsA), inflammatory bowel disease, gastritis, ankylosing spondylitis, ulcerative colitis, pancreatitis, Crohn's disease, celiac disease, multiple sclerosis, systemic lupus erythematosus, lupus nephritis, rheumatic fever, gout, organ or transplant rejection, chronic allograft rejection, acute or chronic graft-versus-host disease, dermatitis including atopic, dermatomyositis, psoriasis, Behcet's diseases, uveitis, myasthenia gravis, Grave's disease, Hashimoto thyroiditis, Sjoergen's syndrome, blistering disorders, antibody-mediated vasculitis syndromes, immune-complex vasculitides, allergic disorders, asthma, bronchitis, chronic obstructive pulmonary disease (COPD), cystic fibrosis, pneumonia, pulmonary diseases including oedema, embolism, fibrosis, sarcoidosis, hypertension and emphysema, silicosis, respiratory failure, acute respiratory distress syndrome, BENTA disease, berylliosis, and polymyositis.

A therapeutically effective dose of Compound A or a pharmaceutical composition thereof includes a dose range from about 100 mg to about 1000 mg, or any particular amount or range therein, in particular, from about 100 mg to about 400 mg, or any particular amount or range therein, of active pharmaceutical ingredient in a regimen of about 1 to about (4×) per day for an average (70 kg) human.

In an alternate embodiment, a therapeutically effective dose of Compound A or a pharmaceutical composition thereof includes a dose range from about 25 mg to about 1000 mg, or any particular amount or range therein, in particular, from about 25 mg to about 400 mg, or any particular amount or range therein, of active pharmaceutical ingredient in a regimen of about 1 to about (4×) per day for an average (70 kg) human.

Compound A may be administered in a single daily dose, or the total daily dosage may be administered in divided doses of two, three and 4× daily.

In one embodiment, the invention comprises a method of treating a disorder or condition that is affected by the inhibition of MALT1 in a subject in need of treatment, comprising administering a therapeutically effective dose ranging from about 25 to about 1000 mg, alternatively from about 25 to about 750 mg, alternatively from about 25 to about 500 mg, alternatively from about 50 to about 1000 mg, alternatively from about 100 to about 1000 mg, alternatively from about 150 to about 350 mg, alternatively from about 200 to about 350 mg, alternatively from about 275 to about 375 mg, alternatively about 300 mg, alternatively at least about 300 mg of 1-(1-oxo-1,2-dihydroisoquinolin-5-yl)-5-(trifluoromethyl)-N-[2-(trifluoromethyl)pyridin-4-yl]-1H-pyrazole-4-carboxamide (Compound A):

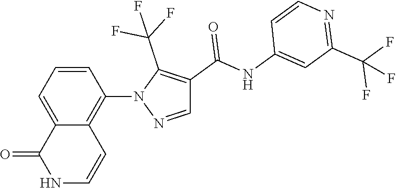 [SMILES: O=C(Nc1ccnc(C(F)(F)F)c1)c1cnn(-c2cccc3c(=O)[nH]ccc23)c1C(F)(F)F]

or pharmaceutically acceptable salt form thereof to said subject.

In one embodiment, the invention comprises 1-(1-oxo-1,2-dihydroisoquinolin-5-yl)-5-(trifluoromethyl)-N-[2-(trifluoromethyl)pyridin-4-yl]-1H-pyrazole-4-carboxamide (Compound A):

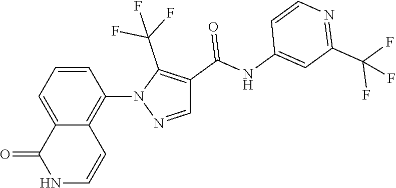 [SMILES: O=C(Nc1ccnc(C(F)(F)F)c1)c1cnn(-c2cccc3c(=O)[nH]ccc23)c1C(F)(F)F]

or a pharmaceutically acceptable salt form thereof, for use in treating a disorder or condition that is affected by the inhibition of MALT1 in a subject, by administration of Compound A or pharmaceutically acceptable salt form thereof, to said subject in an amount of from about 25 to about 1000 mg, alternatively from about 50 to about 1000 mg, alternatively from about 100 to about 1000 mg, alternatively about 50 to about 500 mg, alternatively from about 100 to about 500 mg, alternatively from about 100 to about 400 mg, alternatively from about 150 to about 350 mg, alternatively from about 200 to about 350 mg, alternatively from about 275 to about 375 mg, alternatively about 300 mg, alternatively at least about 300 mg.

In one embodiment, the invention comprises 1-(1-oxo-1,2-dihydroisoquinolin-5-yl)-5-(trifluoromethyl)-N-[2-(trifluoromethyl)pyridin-4-yl]-1H-pyrazole-4-carboxamide (Compound A):

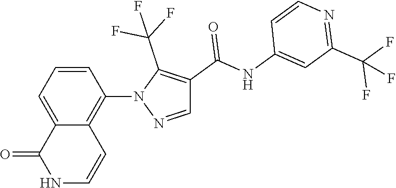 [SMILES: O=C(Nc1ccnc(C(F)(F)F)c1)c1cnn(-c2cccc3c(=O)[nH]ccc23)c1C(F)(F)F]

or a pharmaceutically acceptable salt form thereof, for use in a method of treating a disorder or condition that is affected by the inhibition of MALT1 in a subject, wherein the method comprises administration of Compound A or pharmaceutically acceptable salt form thereof, to said subject in an amount of from about 25 to about 1000 mg, alternatively from about 50 to about 1000 mg, alternatively from about 100 to about 1000 mg, alternatively about 50 to about 500 mg, alternatively from about 100 to about 500 mg, alternatively from about 100 to about 400 mg, alternatively from about 150 to about 350 mg, alternatively from about 200 to about 350 mg, alternatively from about 275 to about 375 mg, alternatively about 300 mg, alternatively at least about 300 mg.

In one embodiment, the invention comprises Compound A, or a pharmaceutically acceptable salt form thereof, for use in treating a disorder or condition that is affected by the inhibition of MALT1 in a subject, wherein Compound A is 1-(1-oxo-1,2-dihydroisoquinolin-5-yl)-5-(trifluoromethyl)-N-[2-(trifluoromethyl)pyridin-4-yl]-1H-pyrazole-4-carboxamide:

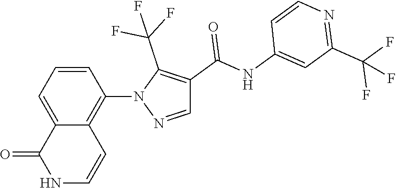 [SMILES: O=C(Nc1ccnc(C(F)(F)F)c1)c1cnn(-c2cccc3c(=O)[nH]ccc23)c1C(F)(F)F]

or a pharmaceutically acceptable salt form thereof, is administered to said subject in an amount of from about 25 to about 1000 mg, alternatively from about 50 to about 1000 mg, alternatively from about 100 to about 1000 mg, alternatively about 50 to about 500 mg, alternatively from about 100 to about 500 mg, alternatively from about 100 to about 400 mg, alternatively from about 150 to about 350 mg, alternatively from about 200 to about 350 mg, alternatively from about 275 to about 375 mg, alternatively about 300 mg, alternatively at least about 300 mg.

In one embodiment, the invention comprises a method of treating a cancer or an immunological disease in a subject in need of treatment, comprising administering a therapeutically effective dose ranging from about 25 to about 1000 mg, alternatively from about 50 to about 1000 mg, alternatively from about 100 to about 1000 mg, alternatively about 50 to about 500 mg, alternatively from about 100 to about 500 mg, alternatively from about 100 to about 400 mg, alternatively from about 150 to about 350 mg, alternatively from about 200 to about 350 mg, alternatively from about 275 to about 375 mg, alternatively about 300 mg, alternatively at least about 300 mg of 1-(1-oxo-1,2-dihydroisoquinolin-5-yl)-5-(trifluoromethyl)-N-[2-(trifluoromethyl)pyridin-4-yl]-1H-pyrazole-4-carboxamide (Compound A):

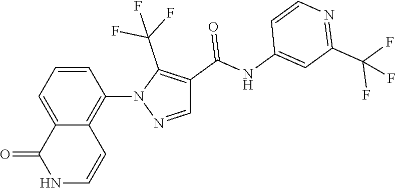 [SMILES: O=C(Nc1ccnc(C(F)(F)F)c1)c1cnn(-c2cccc3c(=O)[nH]ccc23)c1C(F)(F)F]

or a hydrate or a pharmaceutically acceptable salt form thereof to said subject.

In one embodiment, the invention comprises 1-(1-oxo-1,2-dihydroisoquinolin-5-yl)-5-(trifluoromethyl)-N-[2-(trifluoromethyl)pyridin-4-yl]-1H-pyrazole-4-carboxamide (Compound A):

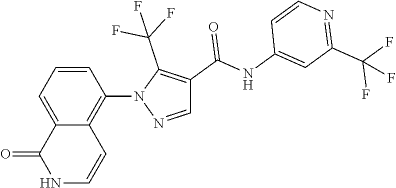 [SMILES: O=C(Nc1ccnc(C(F)(F)F)c1)c1cnn(-c2cccc3c(=O)[nH]ccc23)c1C(F)(F)F]

or pharmaceutically acceptable salt form thereof, for use in treating a cancer or an immunological disease in a subject, by administration of Compound A, or a hydrate or pharmaceutically acceptable salt form thereof, to said subject in an amount of from about 25 to about 1000 mg, alternatively from about 50 to about 1000 mg, alternatively from about 100 to about 1000 mg, alternatively about 50 to about 500 mg, alternatively from about 100 to about 500 mg, alternatively from about 100 to about 400 mg, alternatively from about 150 to about 350 mg, alternatively from about 200 to about 350 mg, alternatively from about 275 to about 375 mg, alternatively about 300 mg, alternatively at least about 300 mg.

In one embodiment, the invention comprises 1-(1-oxo-1,2-dihydroisoquinolin-5-yl)-5-(trifluoromethyl)-N-[2-(trifluoromethyl)pyridin-4-yl]-1H-pyrazole-4-carboxamide (Compound A):

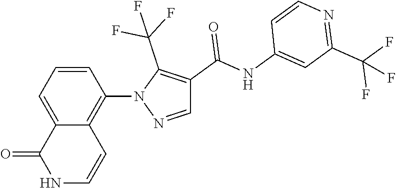 [SMILES: O=C(Nc1ccnc(C(F)(F)F)c1)c1cnn(-c2cccc3c(=O)[nH]ccc23)c1C(F)(F)F]

or pharmaceutically acceptable salt form thereof, for use in a method of treating a cancer or an immunological disease in a subject, wherein the method comprises administration of Compound A, or a hydrate or pharmaceutically acceptable salt form thereof, to said subject in an amount of from about 25 to about 1000 mg, alternatively from about 50 to about 1000 mg, alternatively from about 100 to about 1000 mg, alternatively about 50 to about 500 mg, alternatively from about 100 to about 500 mg, alternatively from about 100 to about 400 mg, alternatively from about 150 to about 350 mg, alternatively from about 200 to about 350 mg, alternatively from about 275 to about 375 mg, alternatively about 300 mg, alternatively at least about 300 mg.

In one embodiment, the invention comprises Compound A, or pharmaceutically acceptable salt form thereof, for use in treating a cancer or an immunological disease in a subject, wherein Compound A is 1-(1-oxo-1,2-dihydroisoquinolin-5-yl)-5-(trifluoromethyl)-N-[2-(trifluoromethyl)pyridin-4-yl]-1H-pyrazole-4-carboxamide:

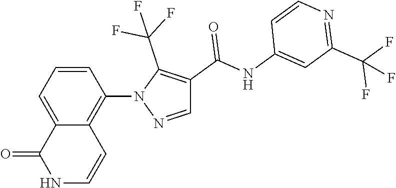 [SMILES: O=C(Nc1ccnc(C(F)(F)F)c1)c1cnn(-c2cccc3c(=O)[nH]ccc23)c1C(F)(F)F]

or a hydrate or pharmaceutically acceptable salt form thereof, is administered to said subject in an amount of from about 25 to about 1000 mg, alternatively from about 50 to about 1000 mg, alternatively from about 100 to about 1000 mg, alternatively about 50 to about 500 mg, alternatively from about 100 to about 500 mg, alternatively from about 100 to about 400 mg, alternatively from about 150 to about 350 mg, alternatively from about 200 to about 350 mg, alternatively from about 275 to about 375 mg, alternatively about 300 mg, alternatively at least about 300 mg.

In another embodiment, the invention is directed to a method of treating a disorder or condition that is affected by the inhibition of MALT1 in a patient in need thereof by administering a daily dose from about 50 to about 500 mg, alternatively from about 100 to about 500 mg, alternatively from about 100 to about 400 mg, alternatively from about 150 to about 350 mg, alternatively from about 200 to about 350 mg, alternatively from about 275 to about 375 mg, alternatively about 300 mg, alternatively at least about 300 mg of 1-(1-oxo-1,2-dihydroisoquinolin-5-yl)-5-(trifluoromethyl)-N-[2-(trifluoromethyl)pyridin-4-yl]-1H-pyrazole-4-carboxamide (Compound A):

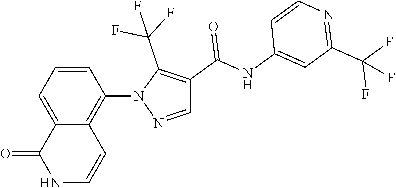 [SMILES: O=C(Nc1ccnc(C(F)(F)F)c1)c1cnn(-c2cccc3c(=O)[nH]ccc23)c1C(F)(F)F]

or a pharmaceutically acceptable salt form thereof to the patient for about 7-21 days. The method may also be repeated, i.e. include one or more repeat cycle. In certain embodiments, the method includes repeat cycles and achieves the following parameters for Compound A: first day of initial administration: Cₘₐₓ about 8.81 μg/mL, AUC about 152 μg·h/mL, and Tₘₐₓ about 4 hours; and first day of repeat cycle: Cₘₐₓ about 55.2 μg/mL, AUC about 1144 μg·h/mL, and Tₘₐₓ about 4 hours.

Yet another embodiment of the invention is a method of treating a disorder or condition that is affected by the inhibition of MALT1 in a subject in need of treatment by administering a dose of from about 50 to about 500 mg, alternatively from about 100 to about 500 mg, alternatively from about 100 to about 400 mg, alternatively from about 150 to about 350 mg, alternatively from about 200 to about 350 mg, alternatively from about 275 to about 375 mg, alternatively about 300 mg, alternatively at least about 300 mg of 1-(1-oxo-1,2-dihydroisoquinolin-5-yl)-5-(trifluoromethyl)-N-[2-(trifluoromethyl)pyridin-4-yl]-1H-pyrazole-4-carboxamide (Compound A):

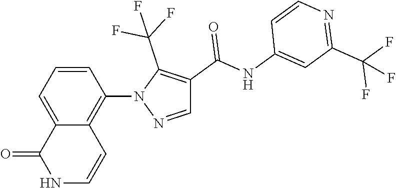 [SMILES: O=C(Nc1ccnc(C(F)(F)F)c1)c1cnn(-c2cccc3c(=O)[nH]ccc23)c1C(F)(F)F]

or pharmaceutically acceptable salt form thereof to said subject, wherein the method comprises administering a dose twice daily for about seven days followed by daily administration of the dose for about 14 days. The method may be repeated, i.e. include a repeat cycle. In some embodiments, the method includes a repeat cycle and achieves the following parameters for Compound A: first day of initial administration: Cₘₐₓ 4.31 μg/mL, Tₘₐₓ about 6 hours; first day of repeat cycle: Cₘₐₓ about 29.2-67 μg/mL, alternatively Cₘₐₓ about 29.2 μg/mL, alternatively Cₘₐₓ about 67 μg/mL, Tₘₐₓ about 3 hours, AUC about 651-1406 μg·h/mL, alternatively AUC about 651 μg·h/mL, alternatively AUC about 1406 μg·h/mL. Alternatively, the method may achieve the following parameters for Compound A on the first day of the repeat cycle: Cₘₐₓ about 29.2 μg/mL, Tₘₐₓ about 3 hours, AUC about 651 μg·h/mL; or Cₘₐₓ about 67 μg/mL, Tₘₐₓ about 3 hours, AUC about 1406 μg·h/mL.

In some embodiments, the invention is a pharmaceutical composition comprising a therapeutically effective amount of Compound A, wherein the pharmaceutical composition achieves a Cₘₐₓ of from about 2 μg/ml to about 120 μg/ml, about 2 μg/ml to about 100 μg/ml, about 2 μg/ml to about 80 μg/ml, about 2 μg/ml to about 60 μg/ml, or about 2 μg/ml to about 20 μg/ml. In some embodiments, the pharmaceutical composition achieves an AUC from about 50 μg·h/ml to about 2500 μg·h/ml, about 50 μg·h/ml to about 2000 μg·h/ml, about 50 μg·h/ml to about 1500 μg·h/ml, about 50 μg·h/ml to about 1000 μg·h/ml, or about 50 μg·h/ml to about 600 μg·h/ml.

In some embodiments, the method comprises administering Compound A at of 300 mg/day QD for continuous 7-21 days

In yet another embodiment, the invention is comprises a method of reducing the T_{reg}/T_{eff} ratio in a patient suffering from a disorder or condition that is affected by the inhibition of MALT1 comprising administering a therapeutically effective amount of 1-(1-oxo-1,2-dihydroisoquinolin-5-yl)-5-(trifluoromethyl)-N-[2-(trifluoromethyl)pyridin-4-yl]-1H-pyrazole-4-carboxamide (Compound A):

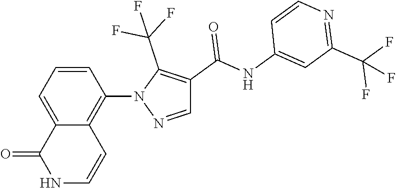 [SMILES: O=C(Nc1ccnc(C(F)(F)F)c1)c1cnn(-c2cccc3c(=O)[nH]ccc23)c1C(F)(F)F]

or pharmaceutically acceptable salt form thereof to said patient. The method may also include the determining the proportions of CD8⁺ T_{eff} and CD4⁺CD25^{hi}FOXP3^{hi} T_{reg} cells. The therapeutically effective amount may be as specified below. In certain embodiments, the therapeutically effective amount is about 50 to about 500 mg, alternatively from about 100 to about 500 mg, alternatively from about 100 to about 400 mg, alternatively from about 150 to about 350 mg, alternatively from about 200 to about 350 mg, alternatively from about 275 to about 375 mg, alternatively about 300 mg, alternatively at least about 300 mg.

### Additional Exemplary Therapeutically Effective Doses and Administration Routes

As noted above, generally, Compound A or a pharmaceutically acceptable salt form thereof may be used in a therapeutically effective dose such as, for example, an amount of from about 25 to about 1000 mg. Additional exemplary suitable therapeutically effective doses and administration routes are described below.

In one embodiment of the invention, the therapeutically effective dose is from about 25 to about 500 mg. In another embodiment of the invention, the therapeutically effective dose is from about 25 to about 200 mg. In yet another embodiment of the invention, the therapeutically effective dose is from about 25 to about 150 mg. In an alternate embodiment of the invention, the therapeutically effective dose is from about 25 to about 250 mg. In an additional embodiment of the invention, the therapeutically effective dose is from about 25 to about 350 mg.

In another embodiment of the invention, the therapeutically effective dose is from about 50 to about 500 mg. In yet another embodiment, the therapeutically effective dose is from about 50 to about 200 mg. In an alternate embodiment the therapeutically effective dose is from about 50 to about 150 mg. In yet another embodiment of the invention, the therapeutically effective dose is from about 100 to about 200 mg.

In another embodiment of the invention, the therapeutically effective dose is about 110 mg. In another embodiment, the therapeutically effective dose is from about 100 to about 400 mg. In yet another embodiment of the invention, the therapeutically effective dose is from about 150 to about 300 mg. In an alternate embodiment of the invention, the therapeutically effective dose is about 200 mg. In another embodiment of the invention, the therapeutically effective dose is from about 100 to about 150 mg.

In another embodiment of the invention, the therapeutically effective dose is from about 150 to about 200 mg. In yet another, the therapeutically effective dose is from about 200 to about 250 mg. In an alternate embodiment, the therapeutically effective dose is from about 250 to about 300 mg. In an additional embodiment, the therapeutically effective dose is from about 300 to about 350 mg. In another embodiment of the invention, the therapeutically effective dose is from about 350 to 400 mg. In another embodiment, the therapeutically effective dose is about 300 mg. In yet another embodiment, the therapeutically effective dose is at least about 300 mg.

In certain embodiments, the therapeutically effective dose of Compound A or a pharmaceutically acceptable salt form thereof may be defined in terms of plasma levels. Accordingly, in an embodiment of the invention, the therapeutically effective dose is an amount sufficient to maintain a plasma level of Compound A from about 2,300 ng/ml to about 9,300 ng/mL. In yet another embodiment of the invention, the therapeutically effective dose is an amount sufficient to maintain a plasma level of Compound A from about 2,320 ng/mL to about 9,280 ng/mL. In another embodiment, the therapeutically effective dose is an amount sufficient to maintain a plasma level of Compound A from about 3,000 ng/ml to about 9,000 ng/mL. In an additional embodiment of the invention, the therapeutically effective dose is an amount sufficient to maintain a plasma level of Compound A from about 3,500 ng/mL to about 8,500 ng/mL.

In another embodiment of the invention, the therapeutically effective dose is an amount sufficient to maintain a plasma level of Compound A from about 4,000 ng/mL to about 8,000 ng/mL. In an alternate embodiment, the therapeutically effective dose is an amount sufficient to maintain a plasma level of Compound A from about 4,000 ng/ml to about 6,000 ng/mL. In yet another embodiment of the invention, the therapeutically effective dose is an amount sufficient to maintain a plasma level of Compound A from about 4,500 ng/ml to about 4,750 ng/mL.

In an embodiment, the therapeutically effective dose is an amount sufficient to maintain a plasma level of Compound A of about 4,640 ng/ml. In another embodiment of the invention, the therapeutically effective dose is an amount sufficient to maintain a plasma level of Compound A from about 4,550 to about 4,700 ng/ml. In yet another embodiment of the invention, the therapeutically effective dose is an amount sufficient to maintain a plasma level of Compound A from about 4,600 to about 4,700 ng/ml. In an alternate embodiment, the therapeutically effective dose is an amount sufficient to maintain a plasma level of Compound A from about 4,550 to about 4,680 ng/ml.

Various dosage cycles may be used to administer the therapeutically effective dose. These dosage cycles may be combined with the various routes of administration, such as those described above.

Compound A may be administered twice a day, daily, every second day. Compound A may also be administered continually for 2 days, alternatively 3 days, alternatively 4 days, alternatively 5 days, alternatively 6 days, alternatively 7 days, alternatively 8 days, alternatively 9 days, alternatively 10 days, alternatively 11 days, alternatively 12 days, alternatively 13 days, alternatively 14 days, alternatively 15 days, alternatively 16 days, alternatively 17 days, alternatively 18 days, alternatively 19 days, alternatively 20 days, alternatively 21 days, alternatively 22 days, alternatively 23 days, alternatively 24 days, alternatively 25 days, alternatively 26 days, alternatively 27 days, or alternatively 28 days.

For instance, in one embodiment of the invention, the therapeutically effective dose is administered twice (two times) a day. In another embodiment, the therapeutically effective dose is administered one time a day. In yet another embodiment of the invention, the therapeutically effective dose is administered on a continuous 28-day cycle. Alternatively, in another embodiment of the invention, the therapeutically effective dose is administered on a continuous 21-day cycle.

In certain embodiments, the cycles of administration may be repeated once, twice, three times, four times, five times, six times, seven times, eight times, or more as necessary. The cycles may also include a rest period during which Compound A is not administered.

In one embodiment, the therapeutically effective dose is from about 150 to about 350 mg, alternatively about 100 to about 300 mg, alternatively about 200 to about 300 mg, or alternatively about 300 mg. This therapeutically effective dose may be administered daily continually for a cycle from 7-21 days. Alternatively, this effective amount may be administered twice daily for a period of 7 days. In certain embodiments, the cycle may be repeated.

In one embodiment of the invention, the therapeutically effective dose is about 300 mg which may be administered daily (QD) continually for 21 day cycle with one or more optional repeat cycles. In some embodiments, the 300 mg QD administration achieves a Cₘₐₓ from about 4 μg/ml to about 12 μg/ml, about 4 μg/ml to about 10 μg/ml, about 4 μg/ml to about 8 μg/ml, about 4 μg/ml to about 6 μg/ml, or about 2 μg/ml to about 12 μg/ml, on day 1 of cycle 1 of administration. In some embodiments, the 300 mg QD administration achieves a Cₘₐₓ of about 40 μg/ml to about 100 μg/ml, about 40 μg/ml to about 80 μg/ml, about 40 μg/ml to about 60 μg/ml, about 40 μg/ml to about 50 g/ml, or about 20 μg/ml to about 100 μg/ml, on day 1 of cycle 2 of administration. In some embodiments, the 300 mg QD administration achieves an AUC from about 50 μg·h/ml to about 250 μg·h/ml, about 50 μg·h/ml to about 200 μg·h/ml, about 50 μg·h/ml to about 150 μg·h/ml, about 50 μg·h/ml to about 100 μg·h/ml, or about 140 μg·h/ml to about 160 μg·h/ml on day 1 of cycle 1 of administration. In some embodiments, the 300 mg QD administration achieves an AUC from about 500 μg·h/ml to about 2500 μg·h/ml, about 500 μg·h/ml to about 2000 μg·h/ml, about 500 μg·h/ml to about 1500 μg·h/ml, about 500 μg·h/ml to about 1100 μg·h/ml, or about 1000 μg·h/ml to about 1200 μg·h/ml on day 1 of cycle 2 of administration.

In other embodiments, the two different administration routes may be combined. For instance, in one embodiment, a dosage regimen which includes administering Compound A daily is combined with subsequent administration twice daily. In another embodiment, a dosage regimen which includes administering Compound A twice daily is combined with subsequent administration daily. Accordingly, in one embodiment, a therapeutically effective dose of about 300 mg is administered twice each day (BID) for about 7 days, followed by administering a therapeutically effective dose of about 300 mg/day daily (QD) for 14 day, with optional repeat cycles of 21 days. In some embodiments, the administration achieves a Cₘₐₓ from about 1 μg/ml to about 10 μg/ml, about 1 μg/ml to about 8 μg/ml, about 1 μg/ml to about 6 μg/ml, about 1 μg/ml to about 4 μg/ml, or about 2 μg/ml to about 4 μg/ml, on day 1 of cycle 1 of administration. In some embodiments, the administration achieves a Cₘₐₓ of about 20 μg/ml to about 100 g/ml, about 20 μg/ml to about 80 μg/ml, about 20 g/ml to about 60 μg/ml, about 20 μg/ml to about 50 μg/ml, or about 30 μg/ml to about 70 μg/ml, on day 1 of cycle 2 of administration. In some embodiments, the administration achieves an AUC from about 500 μg·h/ml to about 2500 μg·h/ml, about 500 μg·h/ml to about 2000 μg·h/ml, about 500 μg·h/ml to about 1500 μg·h/ml, about 500 μg·h/ml to about 1100 μg·h/ml, or about 1000 μg·h/ml to about 1200 μg·h/ml on day 1 of cycle 2 of administration.

Accordingly, in one embodiment, a therapeutically effective dose of about 300 mg is administered twice each day (BID) for about 7 days, followed by administering a therapeutically effective dose of about 300 mg/day daily (QD) until remission.

In one embodiment, the therapeutically effective dose is from about 150 to about 350 mg/day, alternatively from about 100 to about 300 mg/day, alternatively from about 200 to about 300 mg/day, alternatively about 300 mg/day, or alternatively at least about 300 mg/day. This amount may be administered continually for 7-21 days. Alternatively, the therapeutically effective dose is divided in half, said half dose being administered twice (two times) a day and optionally for a period of 7 days. In certain embodiments, the cycle may be repeated.

Thus, in one embodiment of the invention, the therapeutically effective dose is about 300 mg/day which may be administered daily (QD) continually for 7-21 days with one or more optional repeat cycles.

In another embodiment, the therapeutically effective dose is about 300 mg/day and the therapeutically effective dose is divided in half, said half dose being administered twice (two times) a day for seven days followed by daily administration of about 300 mg/day for 14 days, with one or more optional repeat cycle.

In other embodiments, any of the combinations of therapeutically effective dose, administration interval and dosage cycle shown in the Table 1 below may be used:

TABLE 1

| | Adminis- | |
|---|---|---|
| | tration | Dosage |
| Therapeutically effective dose | interval | Cycle |
| about 25 to about 500 mg, alternatively about 50 | one time | |
| to about 500 mg; alternatively about 100 to about | a day | |
| 500 mg; alternatively about 100 to 400 mg; | | |
| alternatively, about 150 to about 300 mg; | | |
| alternatively, about 200 mg; alternatively, about | | |
| 100 to about 150 mg; alternatively, about 150 to | | |
| about 200 mg; alternatively, about 200 to about | | |
| 250 mg; alternatively, about 250 to about 300 | | |
| mg; alternatively, about 300 mg; alternatively, | | |
| about 300 to about 350 mg; alternatively, about | | |
| 350 to about 400 mg | | |
| about 25 to about 500 mg, alternatively about 50 | one time | continuous |
| to about 500 mg; alternatively about 100 to about | a day | 28-day |
| 500 mg; alternatively about 100 to about 400 mg; | | cycle |
| alternatively, about 150 to about 300 mg; | | |
| alternatively, about 200 mg; alternatively, about | | |
| 100 to about 150 mg; alternatively, about 150 to | | |
| about 200 mg; alternatively, about 200 to about | | |
| 250 mg; alternatively, about 250 to about 300 | | |
| mg; alternatively, about 300 mg; alternatively, | | |
| about 300 to about 350 mg; alternatively, about | | |
| 350 to about 400 mg | | |
| about 25 to about 500 mg, alternatively about 50 | one time | continuous |
| to about 500 mg; alternatively about 100 to about | a day | 21-day |
| 500 mg; alternatively about 100 to about 400 mg; | | cycle |
| alternatively, about 150 to about 300 mg; | | |
| alternatively, about 200 mg; alternatively, about | | |
| 100 to about 150 mg; alternatively, about 150 to | | |
| about 200 mg; alternatively, about 200 to about | | |
| 250 mg; alternatively, about 250 to about 300 | | |
| mg; alternatively, about 300 mg; alternatively, | | |
| about 300 to about 350 mg; alternatively, about | | |
| 350 to about 400 mg | | |
| about 25 to about 500 mg, alternatively about 50 | twice (two | |
| to about 500 mg; alternatively about 100 to about | times) | |
| 500 mg; alternatively about 100 to about 400 mg; | a day | |
| alternatively, about 150 to about 300 mg; | | |
| alternatively, about 200 mg; alternatively, about | | |
| 100 to about 150 mg; alternatively, about 150 to | | |
| about 200 mg; alternatively, about 200 to about | | |
| 250 mg; alternatively, about 250 to about 300 | | |
| mg; alternatively, about 300 mg; alternatively, | | |
| about 300 to about 350 mg; alternatively, about | | |
| 350 to about 400 mg | | |
| about 25 to about 500 mg, alternatively about 50 | twice (two | continuous |
| to about 500 mg; alternatively about 100 to about | times) | 28-day |
| 500 mg; alternatively about 100 to about 400 mg; | a day | cycle |
| alternatively, about 150 to about 300 mg; | | |
| alternatively, about 200 mg; alternatively, about | | |
| 100 to about 150 mg; alternatively, about 150 to | | |
| about 200 mg; alternatively, about 200 to about | | |
| 250 mg; alternatively, about 250 to about 300 | | |
| mg; alternatively, about 300 mg; alternatively, | | |
| about 300 to about 350 mg; alternatively, about | | |
| 350 to about 400 mg | | |
| about 25 to about 500 mg, alternatively about 50 | twice (two | continuous |
| to about 500 mg; alternatively about 100 to about | times) | 21-day |
| 500 mg; alternatively about 100 to about 400 mg; | a day | cycle |
| alternatively, about 150 to about 300 mg; | | |
| alternatively, about 200 mg; alternatively, about | | |
| 100 to about 150 mg; alternatively, about 150 to | | |
| about 200 mg; alternatively, about 200 to about | | |
| 250 mg; alternatively, about 250 to about 300 | | |
| mg; alternatively, about 300 mg; alternatively, | | |
| about 300 to about 350 mg; alternatively, about | | |
| 350 to about 400 mg | | |
| about 25 to about 500 mg, alternatively about 50 | | continuous |
| to about 500 mg; alternatively about 100 to about | | 28-day |
| 500 mg; alternatively about 100 to about 400 mg; | | cycle |
| alternatively, about 150 to about 300 mg; | | |
| alternatively, about 200 mg; alternatively, about | | |
| 100 to about 150 mg; alternatively, about 150 to | | |
| 200 mg; alternatively, about 200 to 250 mg; | | |
| alternatively, about 250 to about 300 mg; | | |
| alternatively, about 300 mg; alternatively, about | | |
| 300 to about 350 mg; alternatively, about 350 to | | |
| about 400 mg | | |
| about 25 about to about 500 mg, alternatively | | continuous |
| about 50 to about 500 mg; alternatively about | | 21-day |
| 100 to about 500 mg; alternatively about 100 to | | cycle |
| about 400 mg; alternatively, about 150 to about | | |
| 300 mg; alternatively, about 200 mg; | | |
| alternatively, about 100 to about 150 mg; | | |
| alternatively, about 150 to about 200 mg; | | |
| alternatively, about 200 to about 250 mg; | | |
| alternatively, about 250 to about 300 mg; | | |
| alternatively, about 300 mg; alternatively, about | | |
| 300 to about 350 mg; alternatively, about 350 to | | |
| about 400 mg | | |

The therapeutically effective dose as disclosed herein may be administered in a single daily dose, or the total daily dosage may be administered in divided doses of two, three and 4× daily.

Another embodiment of the invention is a therapeutically effective dose ranging from about 50 to about 1000 mg, alternatively from about 100 to about 1000 mg, alternatively from about 100 to about 400 mg alternatively from about 150 to 300 mg, alternatively about 200 mg, alternatively from about 100 to about 150 mg, alternatively from about 150 to about 200 mg, alternatively from about 200 to about 250 mg, alternatively from about 250 to about 300 mg, alternatively from about 300 to about 350 mg, alternatively from about 350 to about 400 mg of Compound A or a pharmaceutically acceptable salt form thereof for use in treating a disorder or condition that is affected by the inhibition of MALT1.

Yet another embodiment of the invention is use of a therapeutically effective dose ranging from about 50 to about 1000 mg, alternatively from about 100 to about 1000 mg, alternatively from about 100 to about 400 mg alternatively from about 150 to about 300 mg, alternatively about 200 mg, alternatively from about 100 to about 150 mg, alternatively from about 150 to about 200 mg, alternatively from about 200 to about 250 mg, alternatively from about 250 to about 300 mg, alternatively from about 300 to about 350 mg, alternatively from about 350 to about 400 mg of Compound A or a pharmaceutically acceptable salt form thereof for treating a disorder or condition that is affected by the inhibition of MALT1.

An alternate embodiment of the invention is use of a therapeutically effective dose ranging from about 50 to about 1000 mg, alternatively from about 100 to about 1000 mg, alternatively from about 100 to about 400 mg alternatively from about 150 to about 300 mg, alternatively about 200 mg, alternatively from about 100 to about 150 mg, alternatively from about 150 to about 200 mg, alternatively from about 200 to about 250 mg, alternatively from about 250 to about 300 mg, alternatively from about 300 to about 350 mg, alternatively from about 350 to about 400 mg of Compound A or a pharmaceutically acceptable salt form thereof in the manufacture of a medicament for treating a disorder or condition that is affected by the inhibition of MALT1.

An alternate embodiment of the invention is use of a therapeutically effective dose ranging from about 25 to about 1000 mg, alternatively from about 25 to about 500 mg, alternatively from about 25 to about 250 mg, alternatively from about 25 to about 400 mg alternatively from about 25 to about 300 mg, alternatively from about 25 to about 150 mg, alternatively from about 25 to about 200 mg, alternatively from about 25 to about 300 mg, alternatively from about 25 to about 350 mg, alternatively from about 35 to 400 mg, alternatively from about 35 to about 500 mg of Compound A or a pharmaceutically acceptable salt form thereof in the manufacture of a medicament for treating a disorder or condition that is affected by the inhibition of MALT1.

In some embodiments, a method of treating non-Hodgkin's lymphoma (NHL) in a subject comprising administering a therapeutically effective dose of about 100 mg to about 300 mg of Compound A once daily for 7 to 21 continuous day cycle. In some embodiments, the once daily administration cycle is repeated 3-10 times. In some embodiments, the method comprises administering about 100 mg to about 400 mg of Compound A twice daily for 7 days followed by administration of about 100 mg to about 300 mg of Compound A once a day for 14 days. In some embodiments, this cycle is repeated 3-10 times. In some embodiments, the method comprises administering about 100 mg to about 300 mg of Compound A twice daily for 7 days followed by administration of about 100 mg to about 300 mg of Compound A once a day, and the 21 day cycle is repeated until remission.

In some embodiments, a method of treating non-Hodgkin's lymphoma (NHL) in a subject comprises administering a therapeutically effective dose of Compound A, wherein the subject achieves a blood plasma Cₘₐₓ of about 2 μg/ml to about 12 μg/ml on day 1 of administration, and about 40 μg/ml to about 80 g/ml on day 22 of administration. In some embodiments, a method of treating non-Hodgkin's lymphoma (NHL) in a subject comprises administering a therapeutically effective dose of Compound A, wherein the subject achieves an AUC of about 100 μg·h/ml to about 1500 μg·h/ml on day 1 of administration, and about 500 μg·h/ml to about 2000 μg·h/ml on day 22 of administration.

In some embodiments, a method of treating diffuse large B-cell lymphoma (DLBCL) in a subject comprising administering a therapeutically effective dose of about 100 mg to about 300 mg of Compound A once daily for 7 to 21 continuous day cycle. In some embodiments, the once daily administration cycle is repeated 3-10 times. In some embodiments, the method comprises administering about 100 mg to about 400 mg of Compound A twice daily for 7 days followed by administration of about 100 mg to about 300 mg of Compound A once a day for 14 days. In some embodiments, this cycle is repeated 3-10 times. In some embodiments, the method comprises administering about 100 mg to about 300 mg of Compound A twice daily for 7 days followed by administration of about 100 mg to about 300 mg of Compound A once a day, and the 21 day cycle is repeated until remission. In some embodiments, the DLBCL is the activated B cell like (ABC) subtype of diffuse large B-cell lymphoma (DLBCL). In some embodiments, the DLBCL is germinal center B cell like (GCB) subtype of diffuse large B-cell lymphoma (DLBCL). In some embodiments, the DLBCL is non-germinal center B cell like (non-GCB) subtype of diffuse large B-cell lymphoma (DLBCL).

In some embodiments, a method of treating diffuse large B-cell lymphoma (DLBCL) in a subject comprises administering a therapeutically effective dose of Compound A, wherein the subject achieves a blood plasma Cₘₐₓ of about 2 μg/ml to about 12 μg/ml on day 1 of administration, and about 40 g/ml to about 80 μg/ml on day 22 of administration. In some embodiments, a method of treating non-Hodgkin's lymphoma (NHL) in a subject comprises administering a therapeutically effective dose of Compound A, wherein the subject achieves an AUC of about 100 μg·h/ml to about 1500 μg·h/ml on day 1 of administration, and about 500 μg·h/ml to about 2000 μg·h/ml on day 22 of administration. In some embodiments, the DLBCL is the activated B cell like (ABC) subtype of diffuse large B-cell lymphoma (DLBCL). In some embodiments, the DLBCL is germinal center B cell like (GCB) subtype of diffuse large B-cell lymphoma (DLBCL). In some embodiments, the DLBCL is non-germinal center B cell like (non-GCB) subtype of diffuse large B-cell lymphoma (DLBCL).

In some embodiments, a method of treating Waldenström Macroglobulinemia (WM) in a subject comprising administering a therapeutically effective dose of about 100 mg to about 300 mg of Compound A once daily for 7 to 21 continuous day cycle. In some embodiments, the once daily administration cycle is repeated 3-10 times. In some embodiments, the method comprises administering about 100 mg to about 400 mg of Compound A twice daily for 7 days followed by administration of about 100 mg to about 300 mg of Compound A once a day for 14 days. In some embodiments, this cycle is repeated 3-10 times. In some embodiments, the method comprises administering about 100 mg to about 300 mg of Compound A twice daily for 7 days followed by administration of about 100 mg to about 300 mg of Compound A once a day, and the 21 day cycle is repeated until remission.

In some embodiments, a method of treating Waldenström Macroglobulinemia (WM) in a subject comprises administering a therapeutically effective dose of Compound A, wherein the subject achieves a blood plasma Cₘₐₓ of about 2 μg/ml to about 12 μg/ml on day 1 of administration, and about 40 μg/ml to about 80 μg/ml on day 22 of administration. In some embodiments, a method of treating non-Hodgkin's lymphoma (NHL) in a subject comprises administering a therapeutically effective dose of Compound A, wherein the subject achieves an AUC of about 100 μg·h/ml to about 1500 μg·h/ml on day 1 of administration, and about 500 μg·h/ml to about 2000 μg·h/ml on day 22 of administration.

In some embodiments, a method of treating mantle cell lymphoma (MCL)) in a subject comprising administering a therapeutically effective dose of about 100 mg to about 300 mg of Compound A once daily for 7 to 21 continuous day cycle. In some embodiments, the once daily administration cycle is repeated 3-10 times. In some embodiments, the method comprises administering about 100 mg to about 400 mg of Compound A twice daily for 7 days followed by administration of about 100 mg to about 300 mg of Compound A once a day for 14 days. In some embodiments, this cycle is repeated 3-10 times. In some embodiments, the method comprises administering about 100 mg to about 300 mg of Compound A twice daily for 7 days followed by administration of about 100 mg to about 300 mg of Compound A once a day, and the 21 day cycle is repeated until remission.

In some embodiments, a method of treating mantle cell lymphoma (MCL) in a subject comprises administering a therapeutically effective dose of Compound A, wherein the subject achieves a blood plasma Cₘₐₓ of about 2 μg/ml to about 12 μg/ml on day 1 of administration, and about 40 μg/ml to about 80 μg/ml on day 22 of administration. In some embodiments, a method of treating non-Hodgkin's lymphoma (NHL) in a subject comprises administering a therapeutically effective dose of Compound A, wherein the subject achieves an AUC of about 100 μg·h/ml to about 1500 μg·h/ml on day 1 of administration, and about 500 μg·h/ml to about 2000 μg·h/ml on day 22 of administration.

In some embodiments, a method of treating marginal zone lymphoma (MZL) in a subject comprising administering a therapeutically effective dose of about 100 mg to about 300 mg of Compound A once daily for 7 to 21 continuous day cycle. In some embodiments, the once daily administration cycle is repeated 3-10 times. In some embodiments, the method comprises administering about 100 mg to about 400 mg of Compound A twice daily for 7 days followed by administration of about 100 mg to about 300 mg of Compound A once a day for 14 days. In some embodiments, this cycle is repeated 3-10 times. In some embodiments, the method comprises administering about 100 mg to about 300 mg of Compound A twice daily for 7 days followed by administration of about 100 mg to about 300 mg of Compound A once a day, and the 21 day cycle is repeated until remission.

In some embodiments, a method of treating marginal zone lymphoma (MZL) in a subject comprises administering a therapeutically effective dose of Compound A, wherein the subject achieves a blood plasma Cₘₐₓ of about 2 μg/ml to about 12 μg/ml on day 1 of administration, and about 40 μg/ml to about 80 μg/ml on day 22 of administration. In some embodiments, a method of treating non-Hodgkin's lymphoma (NHL) in a subject comprises administering a therapeutically effective dose of Compound A, wherein the subject achieves an AUC of about 100 μg·h/ml to about 1500 μg·h/ml on day 1 of administration, and about 500 μg·h/ml to about 2000 μg·h/ml on day 22 of administration.

In some embodiments, a method of treating follicular lymphoma or transformed follicular lymphoma in a subject comprising administering a therapeutically effective dose of about 100 mg to about 300 mg of Compound A once daily for 7 to 21 continuous day cycle. In some embodiments, the once daily administration cycle is repeated 3-10 times. In some embodiments, the method comprises administering about 100 mg to about 400 mg of Compound A twice daily for 7 days followed by administration of about 100 mg to about 300 mg of Compound A once a day for 14 days. In some embodiments, this cycle is repeated 3-10 times. In some embodiments, the method comprises administering about 100 mg to about 300 mg of Compound A twice daily for 7 days followed by administration of about 100 mg to about 300 mg of Compound A once a day, and the 21 day cycle is repeated until remission.

In some embodiments, a method of treating follicular lymphoma or transformed follicular lymphoma in a subject comprises administering a therapeutically effective dose of Compound A, wherein the subject achieves a blood plasma Cₘₐₓ of about 2 μg/ml to about 12 μg/ml on day 1 of administration, and about 40 g/ml to about 80 μg/ml on day 22 of administration. In some embodiments, a method of treating non-Hodgkin's lymphoma (NHL) in a subject comprises administering a therapeutically effective dose of Compound A, wherein the subject achieves an AUC of about 100 μg·h/ml to about 1500 μg·h/ml on day 1 of administration, and about 500 μg·h/ml to about 2000 μg·h/ml on day 22 of administration.

In some embodiments, a method of treating chronic lymphocytic leukemia (CLL) in a subject comprising administering a therapeutically effective dose of about 100 mg to about 300 mg of Compound A once daily for 7 to 21 continuous day cycle. In some embodiments, the once daily administration cycle is repeated 3-10 times. In some embodiments, the method comprises administering about 100 mg to about 400 mg of Compound A twice daily for 7 days followed by administration of about 100 mg to about 300 mg of Compound A once a day for 14 days. In some embodiments, this cycle is repeated 3-10 times. In some embodiments, the method comprises administering about 100 mg to about 300 mg of Compound A twice daily for 7 days followed by administration of about 100 mg to about 300 mg of Compound A once a day, and the 21 day cycle is repeated until remission.

In some embodiments, a method of treating chronic lymphocytic leukemia (CLL) in a subject comprises administering a therapeutically effective dose of Compound A, wherein the subject achieves a blood plasma Cₘₐₓ of about 2 μg/ml to about 12 μg/ml on day 1 of administration, and about 40 μg/ml to about 80 μg/ml on day 22 of administration. In some embodiments, a method of treating non-Hodgkin's lymphoma (NHL) in a subject comprises administering a therapeutically effective dose of Compound A, wherein the subject achieves an AUC of about 100 μg·h/ml to about 1500 μg·h/ml on day 1 of administration, and about 500 μg·h/ml to about 2000 μg·h/ml on day 22 of administration.

In any of these embodiments above, the subject (patient) may be a human. Additionally, in any of the embodiments above, Compound A is used as a monohydrate form thereof. In another embodiment of the invention, Compound A is used as a hydrate form thereof. In yet an alternate embodiment of the invention, the subject is administered a pharmaceutical composition of Compound A or a pharmaceutically acceptable salt form thereof comprising a pharmaceutically acceptable carrier, a pharmaceutically acceptable excipient, and/or a pharmaceutically acceptable diluent.

In some embodiments, the subject may have received at least 2 prior lines of therapy, including a BTK inhibitor, prior to administration of Compound A. In some embodiments, the subject may have received Ibrutinib prior to administration of Compound A. In some embodiments, the subject may have received first line chemotherapy and at least 1 subsequent line of systemic therapy, including autologous stem cell transplantation (autoSCT), prior to administration of Compound A. In some embodiments, the subject may have received at least 2 prior lines of systemic therapy, including a standard anti CD20 antibody, prior to administration of Compound A. In some embodiments, the subject may have received at least 2 prior lines of systemic therapy, prior to administration of Compound A.

In any of the embodiments of the methods of treatment, Compound A or a pharmaceutically acceptable salt form thereof may be administered via a suitable route of administration. Examples of such suitable routes include but are not limited to oral, parenteral, intramuscular, subcutaneous, intravenous, cutaneous, intramucosal (e.g., gut), intranasal or intraperitoneal routes).

In another embodiment of the present invention, Compound A may be employed in combination with one or more other medicinal agents, more particularly with other anti-cancer agents, e.g. chemotherapeutic, anti-proliferative or immunomodulating agents, or with adjuvants in cancer therapy, e.g. immunosuppressive or anti-inflammatory agents.

Possible combinations of Compound A may include, but are not limited to, BTK (Bruton's tyrosine kinase) inhibitors such as ibrutinib, SYK inhibitors, PKC inhibitors, PI3K pathway inhibitors, BCL family inhibitors, JAK inhibitors, PIM kinase inhibitors, rituximab or other B cell antigen-binding antibodies, as well as immune cell redirection agents (e.g. blinatumomab or CAR T-cells) and immunomodulatory agents such as daratumumab, anti-PD1 antibodies, and anti-PD-L1 antibodies.

All possible combinations of the above-indicated embodiments are considered to be embraced within the scope of this invention.

It will be appreciated that variations to the foregoing embodiments of the invention can be made while still falling within the scope of the invention. Each feature disclosed in this specification, unless stated otherwise, may be replaced by alternative features serving the same, equivalent, or similar purpose. Thus, unless stated otherwise, each feature disclosed is one example only of a generic series of equivalent or similar features.

Reference is now made to the following examples, which illustrate the invention in a non-limiting fashion.

### EXAMPLES

The following examples of the invention are to further illustrate the nature of the invention. It is believed that one of ordinary skill in the art can, using the preceding description and the following illustrative examples, make and utilize the present invention and practice the claimed methods. It should be understood that the following examples do not limit the invention and that the scope of the invention is to be determined by the appended claims.

### Efficacy of Compound A in Lymphomas (Examples 1-5)

The in vivo pharmacodynamic effects and anti-tumor efficacy of Compound A in xenograft mouse models of ABC-DLBCL are shown in the following examples using two xenograft lymphoma models in NSG mice (OCI-LY3 and OCI-LY10 cell lines). Both cell lines are characterized by the constitutive activation of the canonical NF-κB signaling pathway, driven by CARD11 mutation (OCI-LY3) or CD79b mutation (OCI-LY10). In order to reach optimal serum exposures in mouse tumor models, the compound was administered BID in the current studies.

### Materials and Methods

### Mice

NSG mice were obtained from Charles River, France or Jackson Laboratory, USA. All experiments were performed in accordance with The Guide for the Care and Use of Laboratory Animals, the European Communities Council Directives 2010/63/EU, and the USA Animal Welfare Act and were approved by the local ethics committee of Janssen Pharmaceutica N.V., Beerse, Belgium or by the Institutional Animal Care and Use Committee of Janssen R&D, Spring House, PA, USA.

### Cells

The human ABC-DLBCL cell line OCI-LY3 was obtained from Dr. Miguel A Piris, Hospital Universitario Marques de Valdecilla, Santander, Spain. The human ABC-DLBCL cell line OCI-LY-10 was obtained from University Hospital Network, Ontario Cancer Institute. OCY-LY3 cells were maintained at 37° C. in a humidified atmosphere (5% CO₂, 95% air), in RPMI-1640 medium with GlutaMAX™, supplemented with 10% Fetal Bovine Serum (Heat Inactivated at 57° C., and 1% Penicillin-Streptomycin). Each mouse received 1×10⁶ cells in serum-free RPMI-1640 medium or PBS with Matrigel basement matrix in a ratio in a total volume of 0.2 mL. Cells were implanted SC in the right flank using a 1 ml syringe and a 26-gauge needle. The day of tumor implantation was designated as Day 0.

### Compound A

For Examples 1-5, Compound A was formulated as a solution for oral (PO) administration in PEG400 or PEG400 with 10%6:4 linear random copolymer of N-vinylpyrrolidone and vinyl acetate (PVP-VA64). Compound was formulated every 2 weeks, by adding the required volume of PEG400 or PEG400/PVP-VA64 to pre-weighed compound and stirring until dissolved. Formulated compound was stored at room temperature. The free base of Compound A was used for all studies.

### PD Methods

NF-κB signaling regulates the secretion of multiple cytokines, including interleukin-10 (IL-10). MALT1 inhibition results in a decrease of IL-10 transcription and translation as well as secretion. Levels of the human cytokine IL-10 were measured in the serum of OCI-LY3 or OCI-LY10 ABC-DLBCL tumor bearing mice using a Mesoscale Discovery assay (MSD). 25 μL of mouse serum was transferred to an MSD plate (V-Plex Proinflammation Panel I (human) kit) and incubated together with 25 μL diluent 2 (MSD; R51BB-3) for 2 hours at RT followed by a 2-hour incubation with IL-10 antibody solution. Plates were read on a SECTOR imager. Serum human IL-10 levels were correlated to serum compound concentration.

MALT1 protease activity results in the cleavage of negative regulators of the classical NF-κB pathway such as A20, CYLD, RelB, and BCL10. Cleavage of the MALT1 substrate BCL10 was evaluated in tumor samples upon treatment with Compound A. OCI-LY3 or OCI-LY10 tumor samples were analyzed by a BCL10 Mesoscale assay. The assay measures un-cleaved BCL10 which is increased upon MALT1 inhibition. OCI-LY3 or OCI-LY10 tumors were crushed before lysis in Mammalian Protein Extraction Reagent buffer for 30 minutes at 4° C. Centrifugation of the lysate was done and supernatant kept for analysis in the MSD assay. MSD plates (small spot, goat anti-rabbit coated, MSD L45RA-1) were blocked for 1 hour with 3% Bovine serum Albumin (in Tris-buffered saline, 0.1% Tween 20) and labeled with BCL10 antibody (Abcam #33905) to capture the uncleaved BCL10. 25 μg of tumor lysates were transferred to the BCL 10-labeled MSD plate and incubated for 24 hours at 4° C. followed by 2-hour incubation with BCL10 antibody detecting cleaved/uncleaved BCL10 (ab93022) and 2-hour antibody detection. Plates were read on a SECTOR imager.

Tumor volume was calculated using the formula: tumor volume (mm³)=(Dxd²/2); where ‘D’ represents the larger diameter, and ‘d’ the smaller diameter of the tumor as determined by caliper measurements. Tumor volume data was graphically represented as the mean tumor volume±SEM.

Percent tumor growth inhibition (TGI) was defined as the difference between mean tumor volumes of the treated and control group, calculated as

$$\% TGI = [ ( {TV}_{c} - {TV}_{t} ) / {TV}_{c} ] \times 100 ,$$

where TV_{c} is the mean tumor volume of a given control group and TVₜ is the mean tumor volume of the treatment group. As defined by National Cancer Institute criteria, ≥60% TGI is considered biologically significant.

### Data Analysis

Tumor volume or body weight data were graphically represented using Prism software (GraphPad, version 7). Statistical significance for most studies was evaluated for treated groups compared with controls on the last day of treatment. Differences between groups were considered significant when p≤0.05.

Statistical significance was calculated using the linear mixed-effects analysis in R software version 3.4.2 (using Janssen's internally developed Shiny application version 3.3), with treatment and time as fixed effects and animal as random effect. Logarithmic transformation (base 10) was performed if individual longitudinal response trajectories were not linear. The information derived from this model was used to make pairwise treatment comparisons to the control group or between all the treatment groups.

### Example 1

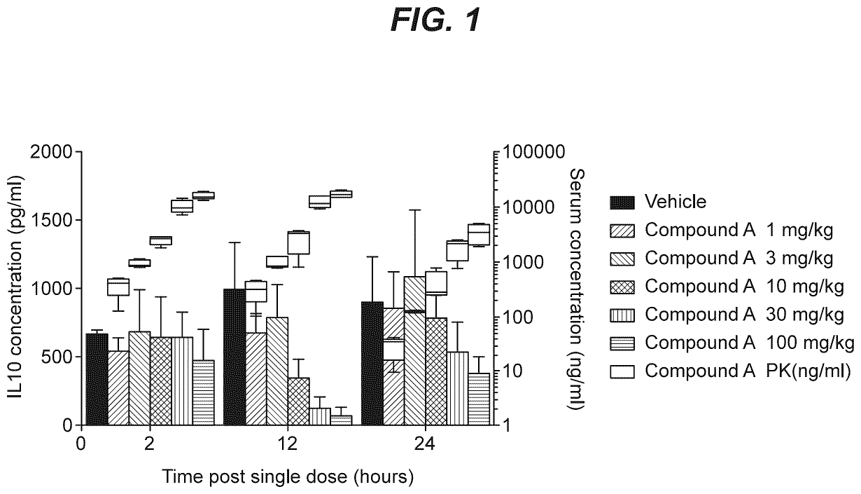

The in vivo pharmacodynamic (PD) activity of Compound A was evaluated in CARD11-mutant OCI-LY3 ABC-DLBCL SC xenografts. Doses of 30 and 100 mg/kg of Compound A completely inhibited or reduced serum IL-10 at 12 hours post dose, while an intermediate inhibition or reduction was observed with 10 mg/kg (FIG. 1). The inhibition or reduction of IL-10 in serum was lost at 24 hours post dose, correlating with declining Compound A serum exposures in mice from 12 to 24 hours.

Serum IL-10 levels are graphed as the mean+/−SD. Male NSG mice were given a single dose of PEG400 vehicle or Compound A (n=5/group). IL-10 levels were correlated to serum Compound A exposures (FIG. 1, plotted as box and whisker plots).

### Example 2

As a more direct PD readout, OCI-LY3 tumor samples were analyzed by a BCL 10 Mesoscale assay, looking for cleavage inhibition of the MALT1 substrate BCL10. The assay measures uncleaved BCL10 in tumor.

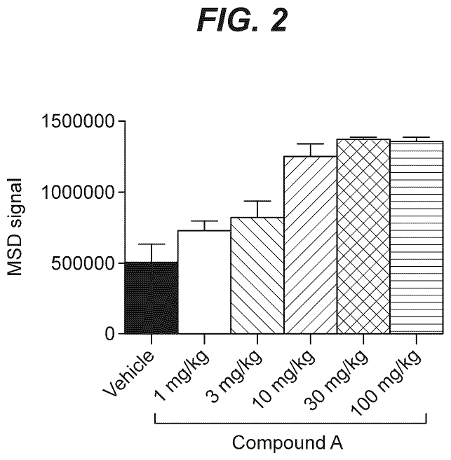

Treatment with Compound A dose-dependently increased the fraction of uncleaved BCL10 and maximum levels were obtained with ≥10 mg/kg of Compound A treatment (FIG. 2).

Tumor uncleaved BCL10 levels are graphed as the mean+SD. Male NSG mice were given a single dose of PEG400 vehicle or Compound A (n=5/group).

### Example 3

Similarly, the in vivo activity of Compound A was evaluated in SC xenografts of CD79b-mutant OCI-LY10 ABC-DLBCL cells. NSG mice bearing OCI-LY10 tumors were treated with a single oral dose of vehicle or Compound A at 3, 10, 30, or 100 mg/kg. In addition, one group of mice was treated with 100 mg/kg of Compound A plus precipitation inhibitor PVP/VA64. Serum samples were collected at 2, 12, and 24 hours post dose and tumor samples were collected 24 hours post dose. Human IL-10 was measured in serum samples using a Mesoscale assay.

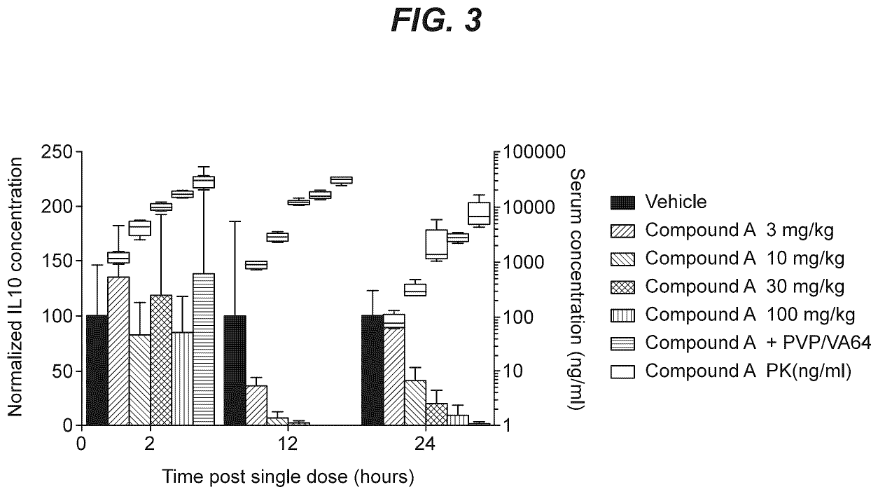

Doses of 30 and 100 mg/kg of Compound A completely inhibited or reduced serum IL-10 levels at 12 hours post dose, while strong inhibition or reduction was observed with 10 mg/kg and an intermediate inhibition or reduction was observed with 3 mg/kg (FIG. 3). Due to larger baseline variability in IL-10 concentration, each time point was normalized to the vehicle group at the indicated time point. The inhibition or reduction of IL-10 in serum rebounded at 24 hours post dose, most likely due to the short t_{1/2} of Compound A in mice as suggested by the lower compound concentrations in serum at 24 hours plotted in FIG. 3. The addition of the precipitation inhibitor PVP/PA64 resulted in increased exposures correlating to increased PD shutdown at 24 hours.

Serum IL-10 levels are graphed as the mean+/−standard deviation. Female NSG mice were given a single dose of PEG400 vehicle or Compound A (n=5/group). IL-10 levels were correlated to serum Compound A exposures (plotted as box and whisker plots).

### Example 4

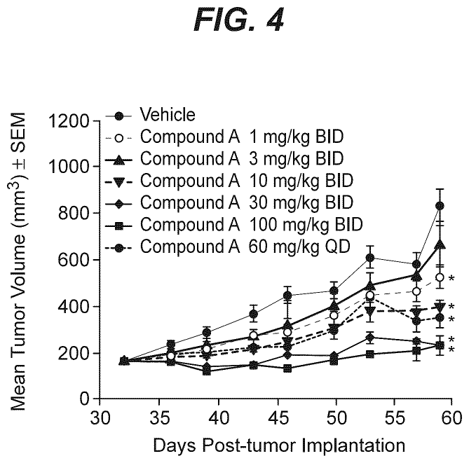

Compound A induced statistically significant antitumor efficacy in the OCI-LY3 DLBCL model. Treatment with 1 or 3 mg/kg BID of Compound A produced very slight antitumor activity with 37% and 19% TGI observed, respectively, as compare with vehicle treated control mice. At 10 mg/kg BID, Compound A was efficacious, with TGI reaching 53% and p=0.0015 (compared to the control group; In vivo Longitudinal Data Analysis 3.3) (FIG. 4). Efficacy of 30 and 100 mg/kg was comparable and resulted in 72% TGI for both doses, when compared to the PEG400-treated control group.

In order to assess the importance of steady drug administration every 12 hours during efficacy studies, there was an additional group treated with 10 mg/kg of Compound A included. This group was however dosed using a 8/16 hr split schedule, instead of 12/12 hr. Despite different dosing schedules, both groups treated with 10 mg/kg reached similar TGI (57%, data not shown)

In order to assess the impact on dosing frequency, Compound A was tested at 60 mg/kg QD, with statistically significant 57% TGI being reached as compare to the vehicle-treated control group. This was slightly lower than that achieved with 30 mg/kg BID (72% TGI). This indicates that trough drug levels are important for efficacy.

Group tumor volumes are graphed as the mean+/−SEM. Mice were implanted SC on the right flank on Day 0. After 32 days post implantation, when tumors were established (mean rumor volume 164 mm³), mice were randomized into experimental groups and dosed orally twice or once daily for 4 weeks with PEG400 vehicle or Compound A Statistical analyses of treated vs. Vehicle groups calculated on Day 59 using LME analysis in R software version 3.4.2 (Janssen Shiny application version 3.3), and were considered significant when *p<0.05.

SEM, standard error of the mean; BID, twice daily; QD, once daily; LME, linear mixed-effects.

### Example 5

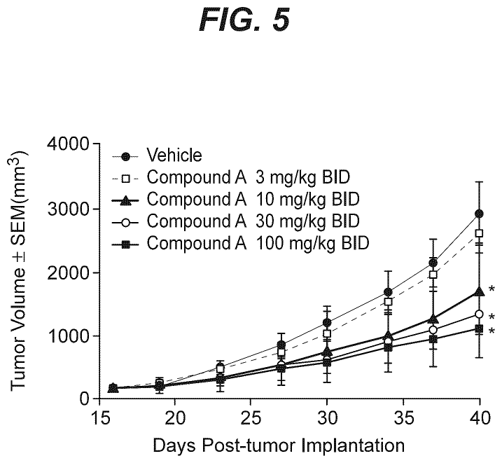

Compound A induced statistically significant antitumor efficacy in the OCI-LY10 DLBCL model. Treatment with 3 mg/kg BID Compound A produced only 11% TGI that was not statistically significant, whereas dose levels of 10, 30, and 100 mg/kg BID elicited 41%, 54%, and 62% TGI, respectively as compared with vehicle treated control animals (p<0.01 and p<0.001, p<0.001) (FIG. 5).

Group tumor volumes are graphed as the mean+SEM. Mice were implanted SC on the right flank on Day 0. After 15 days post implantation, when tumors were established (mean tumor volume 169 mm³), mice were randomized into experimental groups and dosed orally twice daily beginning Day 16 for 3.5 weeks with PEG400 vehicle or Compound A (n=10/group) Statistical analyses of treated vs. Vehicle groups were calculated on Day 40 using LME analysis in R software version 3.4.2 (Janssen Shiny application version 3.3) and were considered significant when *p<0.05.

### Discussion of Examples 1-5

The goal of these studies was to assess the pharmacodynamic and antitumor activity of orally administered Compound A, an allosteric inhibitor of MALT1 protease, in CD79b- and a CARD11-mutant ABC-DLBCL xenograft models with constitutive activation of the canonical NF-κB signaling. Compound A was assessed at dose levels from 1 to 100 mg/kg (BID or QD) in established OCI-LY3 and OCI-LY10 xenograft models in NSG, mice, and was well tolerated.

In the OCI-LY3 tumor model, a single dose of 30 and 100 mg/kg of Compound A completely inhibited or reduced serum IL-10 at 12 hours post dose, while an intermediate inhibition or reduction was observed with 10 mg/kg. The inhibition or reduction of IL-10 in serum was lost at 24 hours post dose, correlating with declining Compound A serum exposures in mice from 12 to 24 hours. Compound A also inhibited the ability of MALT1 to cleave BCL 10 substrate in tumors, with maximal increased fraction of uncleaved BCL10 observed at dose levels ≥10 mg/kg.

In the OCI-LY10 tumor model, single doses of 30 and 100 mg/kg of Compound A completely inhibited or reduced serum IL-10 at 12 hours post dose, while strong inhibition or reduction was observed with 10 mg/kg and an intermediate inhibition or reduction was observed with 3 mg/kg.

In the established OCI-LY3 DLBCL model, Compound A induced statistically significant antitumor efficacy at 10, 30, and 100 mg/kg dose levels given twice daily, with 53%, 72%, and 72% TGI observed, respectively, as compared with vehicle control-treated mice. These data were comparable to previous efficacy studies in the OCI-LY3 model, where efficacy with 10 mg/kg BID of Compound A elicited 51-76% TGI, 30 mg/kg BID 60-86% TGI, and 100 mg/kg 62-89% TGI. Overall, in 3 independent OCI-LY3 antitumor efficacy studies, a dose of 10 mg/kg BID of Compound A produced a mean of 60% TGI and is therefore considered the minimum efficacious dose.

In the established OCI-LY10 model, statistically significant antitumor activity was elicited with Compound A treatment, with 41%, 54%, and 62% TGI observed, respectively with 10, 30, and 100 mg/kg of Compound A as compared to vehicle treated control animals.

### Dosing Rationale

Human dosing parameters for Compound A were selected in accordance with the S9 guidance for anticancer pharmaceuticals using nonclinical data (Guidance for Industry). Pharmacokinetic modeling and simulations were performed to predict the dose in humans that would give an observed plasma concentration immediately prior to next dose administration (C_{trough}) of 4640 ng/ml at steady state. This C_{trough} corresponds to the observed trough concentration (total) of 2,202 ng/mL following the lowest efficacious dose of 10 mg/kg BID in a tumor-bearing mouse efficacy study (after accounting for the difference in protein binding between mice and human). According to the tested scenario, the minimum predicted efficacious dose is approximately 110 mg given once daily.

The pharmacokinetic modeling was used based on available non-clinical pharmacokinetic data and allometric scaling. In addition, modeling using GastroPlus and SimCYP was also used to guide PK parameter prediction.

### Example 6

### IL-6/10 Secretion Assay Using DLBCL Cell Line

NF-κB signaling regulates the secretion of multiple cytokines, including interleukin (IL)-6 and IL-10. Libermann T A et al. Mol Cell Biol. 1990; 10(6):3155-3162; and Cao S, et al. J Biol Chem. 2006; 281(36):26041-26050. Secretion of the cytokines IL-6 and IL-10 by OCI-LY3 ABC-DLBCL cells was measured using a MesoScale Discovery (MSD) assay. Using the mesoscale assay from supernatants of OCI-LY3 cells, the IC₅₀ value of Compound A across 16 independent experiments was 114±41 nM for IL-6 and 77±24 nM for IL-10 (see Table 2 below).

### Cellular Cleavage of MALT1 Substrates

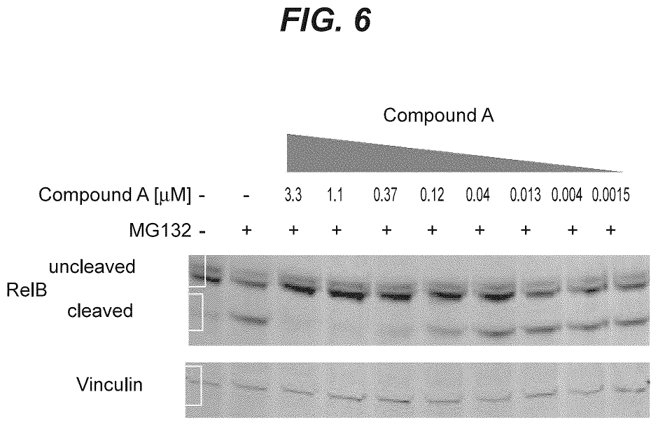

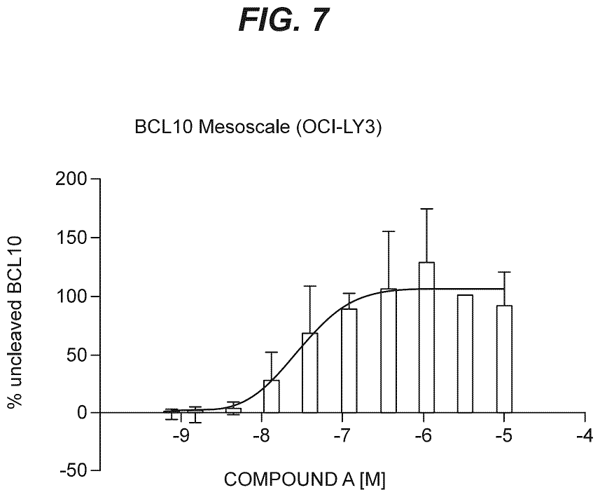

MALT1 protease activity results in the cleavage of inhibitors of the canonical NF-κB pathway such as A20, CYLD, RelB, and BCL10. Cleavage of 2 MALT1 substrates, BCL 10 and RelB, was examined after MALT1 inhibitor treatment. Hachmann J, et al. Biochimie. 2016; 122:324-338. OCI-LY3 cells were treated with various doses of Compound A for 5 hours. The cell-permeable proteasome inhibitor MG132 was added for 4 hours to stabilize cleaved RelB. MALT1 inhibition with Compound A resulted in inhibition of RelB cleavage in OCI-LY3 cells in vitro (FIG. 6). The IC₅₀ value of Compound A across three independent experiments determined with capillary Western (automated Western blot machine Peggy Sue by Protein Simple) was 69.31±9.6 nM. MALT1 inhibition by Compound A in OCI-LY3 cells resulted in a decrease of BCL10 cleavage and, therefore, an increase of uncleaved BCL10 (FIG. 7). The calculated IC₅₀ value for BCL10 (B-cell chronic lymphocytic leukemia/lymphoma 10) cleavage inhibition of Compound A across four independent experiments was 49.6±30.7 nM analyzed with capillary Western (automated Western blot machine Peggy Sue by Protein Simple) and 27.8±13.1 nM analyzed with Mesoscale (FIG. 7).

Table 2 below summarizes in vitro cellular activity of Compound A in OCI-LY3 cells.

TABLE 2
In vitro cellular activity

| | | Compound A IC₅₀ (nM) |
|---|---|---|
| MALT1 enzyme | | 74 ± 23 |
| OCI-LY3 | IL-10 | 77 ± 24 |
| | Proliferation | 496 ± 262 |
| | RelB | 69.31 ± 9.6 |
| | BCL10 | 27.8 ± 13.1 |
| | IL-6 | 114 ± 41 |

### Example 7

### Whole-Gene Transcriptomics in DLBL Cell Lines

Treatment with Compound A resulted in clear dose-dependent gene expression changes in OCI-LY3 cells harboring Myd88 and CARD11 mutations. Similar changes (although to a lesser extent) were seen in TMD8 cells harboring Myd88 and CD79b mutations. TMD8 cell lines overexpressing BTK C481S or CARD11 L244P mutations showed similar gene expression changes to parental TMD8 but at slightly higher concentrations of Compound A. The C481S mutation in BTK prevents the covalent binding of ibrutinib to BTK, leading to resistance. Mutations in the coiled-coil domain of CARD11, e.g. L244P, lead to CBM complex activation in the absence of extracellular stimulus downstream of BTK and therefore result in resistance to ibrutinib treatment. Wilson W H, et al. Nat Med. 2015, 21, 922-926. In contrast, treatment with ibrutinib only resulted in gene expression changes in TMD8 cell lines, while OCI-LY3 cells and TMD8 cell lines expressing the known ibrutinib resistance mutations (BTK C481S or CARD11 L244P) did not show any gene expression changes.

### Inhibition of Translocated API2-MALT1

In 43% of the MALT lymphoma cases, a genetic abnormality is identified that arises by a translocation of API2 and MALT1 genes resulting in the API2-MALT1 fusion oncoprotein. API2-MALT1 expression alone can stimulate IκB kinase (IKK) complex activation and induce NF-κB signaling. See Rosebeck S, et al. Future Oncol. 2011, 7, 613-617.

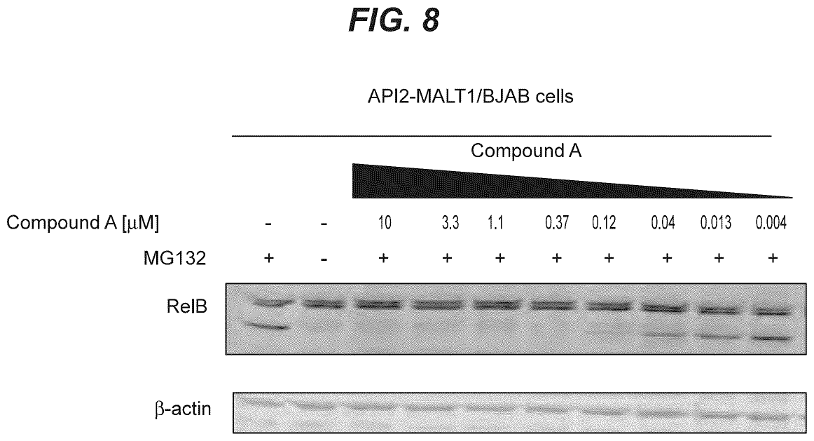

MALT lymphoma may be a secondary indication of a MALT1 inhibitor. BJAB cells overexpressing API2-MALT1 were used to assess whether Compound A inhibits API2-MALT1. Compound A dose-dependently inhibited RelB cleavage by overexpressed API2-MALT1 in BJAB cells (FIG. 8).

### Example 8: Scaffolding Function of MALT1

Besides the protease function, MALT1 has a scaffolding function in NF-κB signaling by recruiting signaling proteins. The downstream effect of the scaffolding function of MALT1 can be assessed by looking at phosphorylation of IκBα. In resting conditions, IκBα forms a complex with NF-κB which prevents its nuclear translocation and therefore its function as a transcription factor. Upon stimulation, IκBα is phosphorylated and signalled for degradation by the proteasome, leading to the release of NF-κB. It has been shown that the CBM complex and scaffolding function of MALT1 is required for phosphorylation of IκBα. See Turvey S E et al. J Allergy Clin Immunol. 2014, 134, 276-284.

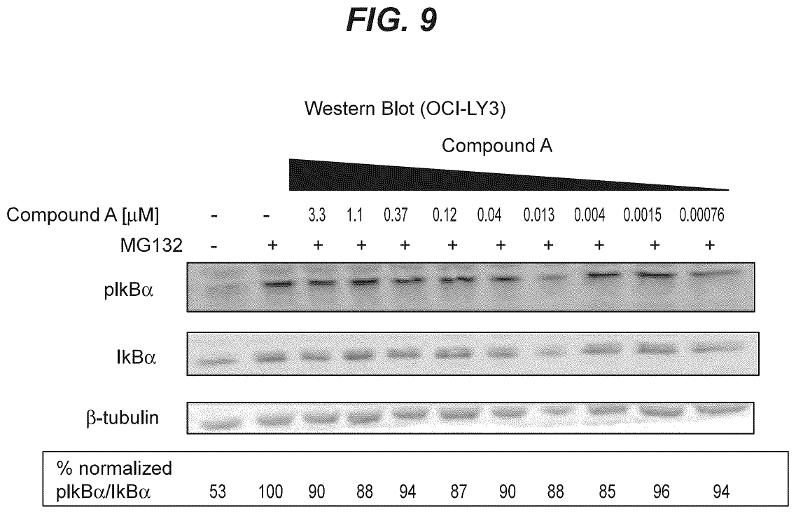

Treatment of OCI-LY3 cells with Compound A did not inhibit phospho-IκBα leading to the conclusion that Compound A did not affect the scaffolding function of MALT1 under unstimulated conditions (FIG. 9). The phospho-IκBα (pIκBα) signal was quantified as percent of DMSO control and normalized against β-tubulin. No changes in IκBα phosphorylation were detected upon treatment with Compound A.

### Example 9: Inhibition of Cancer Cell Proliferation

### DLBCL Cell Lines

To determine the anti-proliferative activity of Compound A, the following panel of 10 B-cell lymphoma lines was treated with different doses of Compound A: ABC-DLBCL cell lines (OCI-LY3, OCI-LY10, TMD8, HBL-1, HLY-1, and U-2932), GCB-DLBCL cell lines (OCI-LY1, OCI-LY7, and SU-DHL-4). Four ABC-DLBCL cell lines with activating mutations in the canonical NFκB pathway were evaluated (OCI-LY3 (CARD11, MYD88 & A20 mutations), TMD8, HBL1, and OCI-LY10 (CD79B & MYD88 mutations)), which are generally sensitive to NF-κB pathway inhibition. None of the GCB-DLBCL cell lines harbor mutations in the NF-κB pathway. The GCB-DLBCL cell lines served as a negative control to exclude compounds with general cytotoxic effects. In addition, the MCL cell line REC-1 was evaluated which is known to be dependent on the NF-κB pathway and was shown to be sensitive to ibrutinib.

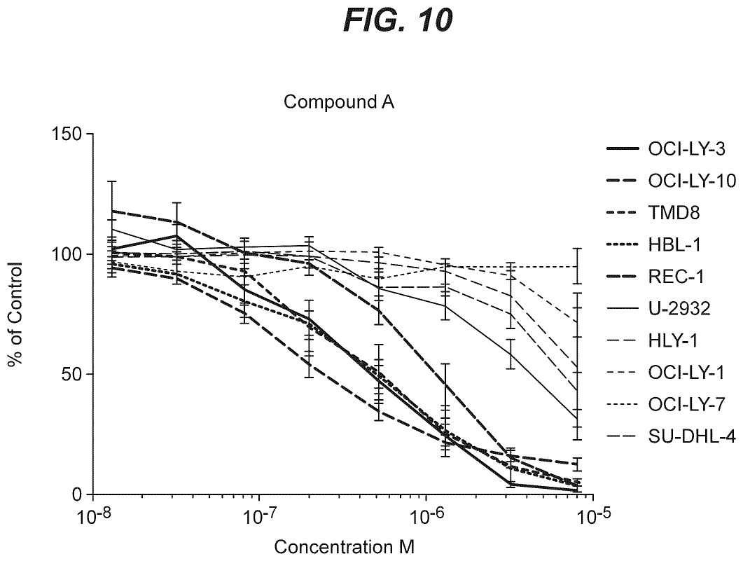

CD79b- and CARD11-mutant ABC-DLBCL cell lines displayed anti-proliferative activity with sub-micromolar IC₅₀ values after treatment with Compound A as shown in FIG. 10. Table 3 shows the antiproliferation (IC₅₀) after 8 days of treatment with Compound A. Furthermore, antiproliferative activity was observed in the MCL cell line REC1. GCB-DLBCL cell lines or ABC-DLBCL lines with A20 homozygous mutations or A20/TAK1 double mutations showed much higher IC₅₀ values or were completely insensitive to Compound A up to 10 μM. At concentrations ≥20 μM general cytotoxicity was observed. Anti-proliferative effects were stronger after 8 days incubation compared to 4 days which is consistent with data obtained for ibrutinib.

TABLE 3
Anti-proliferative activity of Compound
A in a DLBCL cell line panel

| Cell Line | IC₅₀ μM |
|---|---|
| OCI-LY3 (Myd88/CARD11 A20 mut) | 0.496 |
| OCI-LY10 (CD79b/Myd99/A20 mut) | 0.332 |
| TMD8 (CD79b/Myd88/A20 mut) | 0.635 |
| HBL1 (CD79b/Myd88/A20 mut) | 0.547 |
| Rec1 (Ibrutinib sensitive MCL) | 1.17 |
| HLY-1 (Myd88/CARD11/homoz A20 mut) | 8.8 |
| U2932 (A20/TAK1 mut) | 3.9 |
| OCI-Ly1 (no NF-κB mut) | >10 |
| OCT-Ly7 (no NF-κB mut) | >10 |
| SU-DHL4 (no NF-κB mut) | 6.0 |

### Engineered Cell Line Models

TMD8 cell lines harbouring CD79b and Myd88 mutations are sensitive to BTK and MALT1 inhibition. Acquired BTK C481S mutations have been associated with ibrutinib resistance in CLL patients. Ahn I E, et al. Blood. 2017, 129, 1469-1479. Overexpression of the BTK C481S mutant in TMD8 cells results in resistance to ibrutinib up to 100 nM, which mimics findings in mimicking the findings in ibrutinib-resistant chronic lymphocytic leukemia patients. Similarly, overexpression of the CARD11 L244P mutant in TMD8 cells results in resistance to ibrutinib up to 100 nM. The antiproliferative activity of Compound A was similar in BTK C481S-mutant and CARD11 L244P-mutant TMD8 cells, and only a minor IC₅₀ shift was observed compared with wild-type TMD8 cell lines, supporting the hypothesis that a MALT1 inhibitor is a potential treatment option for ibrutinib-resistant malignancies. Concentrations higher than 100 nM ibrutinib can lead to off-target activity and unspecific cell killing.

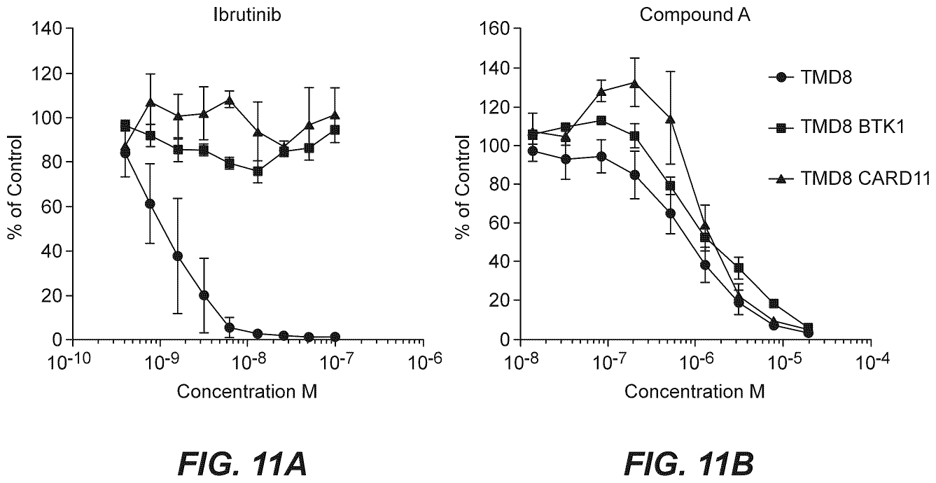

Compound A activity remained similar in BTK C481S and CARD11 L244P mutant TMD8 cells and only a minor IC₅₀ shift compared to wild-type TMD8 cell lines was observed supporting the hypothesis that a MALT1 inhibitor is a valuable treatment option for ibrutinib-resistant tumors (FIGS. 11A and 11B).

### Growth Inhibition Across Various Cancer Indications

To assess the activity of Compound A on broad tumor cell viability, Compound A was tested in a panel of 91 tumor cell lines from multiple indications with different growth properties and genetic backgrounds. The panel represents cancer cell lines from more than 18 different tumor indications, including breast, colon, lung, ovarian, and blood cancers. None of the blood cancer cell lines are derived from ABC-DLBCL patients and do not harbor the known NF-κB-activating mutations. There were no single cell lines showing sensitivity to Compound A up to 20 μM.

### CARD11 Mutant DLBCL Model

The antitumor efficacy of Compound A was evaluated in established subcutaneous (SC) OCI-LY3 human CARD11-mutant xenografts in male NSG mice and in established SC OCI-LY10 human CD79b-mutant xenografts in female NSG mice. To determine the anti-proliferative activity of Compound A, the mice were treated with 1 mg/kg, 3 mg/kg, 10 mg/kg, 30 mg/kg, 60 mg/kg, and 100 mg/kg for 28 days. The efficacy of Compound A treatment in OCI-LY3 cells was assessed by comparing changes in the mean tumour volume as a function of time (see FIG. 4 and Table 4 below).

TABLE 4
Efficacy of Compound A in OCI-LY3 cells

| | TGI (%) at day 28 |
|---|---|
| Compound A (1 mg/kg, bid) | 46 |
| Compound A (3 mg/kg, bid) | 23 |
| Compound A (10 mg/kg, bid) | 66 |
| Compound A (30 mg/kg, bid) | 90 |
| Compound A (100 mg/kg, bid) | 90 |
| Compound A (60 mg/kg, qd) | 71 |

To assess the effect on dosing frequency, Compound A was tested at 60 mg/kg once daily with statistically significant 57% TGI reached, compared with the vehicle-treated control group. The TGI at 60 mg/kg once daily was slightly lower than that achieved with 30 mg/kg twice daily (72% TGI).

In addition, IL-10 serum levels were measured as function of time post single dose administration of various dosages of Compound A. Furthermore, uncleaved BCL10 levels in the tumors were measured at twenty-four hours after administration of the various dosages of Compound A. These data show administration of Compound A leads to potent in vivo pharmacodynamic shutdown and tumor growth inhibition in CARD11 mut DLBCL model.

### Example 10: Assessment of Effectiveness of Compound a in Diffuse Large B-Cell Lymphoma Xenografts in NSG Mice

### Cell Culture

As in Examples 1-5 above, the human ABC-DLBCL cell line OCI-LY3 was obtained from Dr. Miguel A Piris, Hospital Universitario Marques de Valdecilla, Santander, Spain. The human ABC-DLBCL cell line OCI-LY10 was obtained from University Hospital Network, Ontario Cancer Institute. OCY-LY3 cells were maintained at 37° C. in a humidified atmosphere (5% CO₂, 95% air), in RPMI-1640 medium with GlutaMAX™, supplemented with 10% Fetal Bovine Serum (Heat Inactivated at 57° C.), and 1% Penicillin-Streptomycin. Each mouse received 1×10⁷ OCI-LY3 cells in serum-free RPMI-1640 medium or PBS with Matrigel basement matrix in a 1:1 ratio in a total volume of 0.2 mL or 0.1 mL. Cells were implanted SC in the right flank using a 1 mL syringe and a 26-gauge needle. The day of tumor implantation was designated as Day 0.

OCY-LY10 cells were maintained at 37° C. in a humidified atmosphere (5% CO₂, 95% air), in RPMI-1640 medium with GlutaMAX™, supplemented with 10% Fetal Bovine Serum (Heat Inactivated at 57° C.) and 1% Penicillin-Streptomycin. Each mouse received 1×10⁶ OCI-LY10 cells in serum-free RPMI-1640 medium with Matrigel in 1:1 ratio in a total volume of 0.2 mL. Cells were implanted SC in the right flank using a 1 mL syringe and a 26-gauge needle. The day of tumor implantation was designated as Day 0.

### Study Design

The doses selected for Compound A anti-tumor efficacy studies were based on single-dose PK/PD data. Study designs are summarized in Table 5 and described below.

TABLE 5
Study design and treatments

| | | | Mean tumor | | |
|---|---|---|---|---|---|
| | | | volume at | Treatment | Treatment groups |
| Study | Tumor Model | Study Type | randomization | Duration | with Compound A |
| 1 | OCI-LY3 in NSG | PK/PD | 690 mm³ | Single dose | 0, 1, 3, 10, 30, 100 |
| | male mice | | | | mg/kg in PEG400; |
| | | | | | 10 mL/Kg (n = 5) |
| 2 | OCI-LY10 in NSG | PK/PD | 568 mm³ | Single dose | 0, 3, 10, 30, 100 |
| | female mice | | | | mg/kg in PEG400; |
| | | | | | 10 mL/Kg (n = 5) |
| 3 | OCI-LY3 in NSG | Efficacy | 164 mm³ | 4 weeks | 0, 1, 3, 10, 30, 100 |
| | male mice | | (Day 32) | | mg/kg BID, 60 |
| | | | | | mg/kg QD in |
| | | | | | PEG400, 5 mL/Kg |
| | | | | | BID, 5 mL/Kg QD; |
| | | | | | (n = 10) |
| 4 | OCI-LY10 in NSG | Efficacy | 164 mm³ | 3.5 weeks | 100 mg/kg BID in |
| | female mice | | (Day 15) | | PEG400 + PVP VA, |
| | | | | | 5 mL/Kg; (n = 5) |

PK, pharmacokinetic; PD, pharmacodynamic; BID, bis in die (twice daily), QD “quaque die” (once daily).

For PD, when tumors reached 550-750 mm³ animals were assigned randomly into the treatment groups and treated as presented in Table 5. Dose volumes were adjusted to individual body weights at 10 mL/kg. For both PD studies, serum was collected at 2 and 12 hours post treatment from the retro-orbital sinus. At 24 hours post treatment, animals were bled via decapitation and tumors were excised, weighed, and divided for bioanalysis and ex vivo PD analysis. Blood was centrifuged at 10,000 rpm and serum divided for bioanalysis and PD analysis.

For efficacy studies, the dose volumes of Compound A or vehicle controls were adjusted to individual body weights at 5 mL/kg BID, for a total of 10 mL/kg/day, or 5 mL/kg QD.

For study 3, animals were randomly assigned into treatment groups, when the OCI-LY3 mean tumor volume was 164 mm³ (Day 32 post-tumor cell implantation). Treatment was initiated on the day of randomization (Day 32). After 28 days of treatment on Day 60, serum was collected at 2, 4, and 12 hours post last dose for the bid treated groups or 2, 4 and 24 hours post last dose for qd treated groups in cohorts of 5 mice. Blood was collected from the retro-orbital sinus and submitted for bioanalytical analysis.

For study 4, study animals were randomly assigned into treatment groups, when the mean OCI-LY10 tumor volume reached 169 mm³ (Day 15 post-tumor cell implantation). Treatment was initiated the day after randomization (Day 16). Treatment for the antitumor efficacy study lasted for 24 days until Day 40, when most animals in the Vehicle control group reached ethical tumor volume limits. Remaining mice in Compound A treated groups continued treatment until Day 44 when serum was collected at 2, 4, 12, and 24 hours post last dose in cohorts of 5 mice. Blood was collected from the retro-orbital sinus and submitted for bioanalytical analysis.

### In Vivo Serum and Tumor Levels of Compound A

The exposure of Compound A was evaluated in SC xenografts of OCI-LY3 in NSG mice. Exposure after a single oral administration of vehicle or Compound A at 1, 3, 10, 30, or 100 mg/kg was evaluated. Serum samples were collected at 2, 12, and 24 hours post dose and tumor samples were collected 24 hours post dose. Tumor and serum exposures are presented in Table 6.

TABLE 6
Mean (SD) serum and tumor levels in male OCI-LY3 tumor-bearing NSG
mice following a single oral dose of Compound A

| | | Compound A Concentration | | | | Tumor: serum |
|---|---|---|---|---|---|---|
| | | Serum (ng/ml) | | | Tumor (ng/g) | ratio |
| Dose | | Time post dose (h) | | | | |
| (mg/kg) | N | 2 | 12 | 24 | 24 | 24 |
| 100 | 5 | 3,734 (1,633) | 4,118 (1,322 | 1,062 (514) | 1,180 (223)^{b} | 0.98 (0.19)^{b} |

N, number of mice; SD, standard deviation.
Note:
Compound A dosed as PEG400 solution
^{a}Lower level of quantification (LLOQ) serum = 25.0 ng/ml; LLOQ tumor = 44.4-57.5 ng/g.
^{b}N = 4; no tumor sample for one mouse 24 hours post dose.

Exposure after multiple oral administrations of vehicle or Compound A at 10, 30, and 100 mg/kg twice daily or 60 and 100 mg/kg once daily was evaluated. Serum samples were collected at 7 and 12 hours post dose and tumor samples were collected 12 hours post last dose from the twice-daily-treated animals. From once-daily-treated animals, serum samples were collected at 4, 12, and 24 hours post dose and tumor samples were collected at 24 hours post last dose. Serum and tumor Compound A levels are shown in Table 7 (twice-daily dosing) and Table 8 (once-daily dosing).

TABLE 7
Mean (SD) serum and tumor levels in male OCI-LY3 tumor-bearing NSG
mice following twice-daily oral doses of Compound A

| | Total | | | | | Tumor | Tumor: |
|---|---|---|---|---|---|---|---|
| | daily | | | | | Concentration | serum |
| Dose | | | | Serum concentrationª (ng/ml) | | (ng/g) | ratio |
| (mg/ | dose | | | Total time post dose (h) | | | |
| kg) | (mg) | N | Day | 7 | 12 | 12 | 12 |
| 10 | 20 | 6 | 1^{b} | 3,260 (708) | 2,442 (390) | — | — |
| 10 | 20 | 6 | 4^{c} | 3,760 (573) | — | — | — |
| 10 | 20 | 6 | 5^{d} | — | 2,202 (313) | 2,161 (962) | 1.0 (0.4) |
| 30 | 60 | 6 | 1^{b} | 9,967 (1,739) | 9,000 (2,655) | — | — |
| 30 | 60 | 6 | 4^{c} | 7,425 (1,880) | — | — | — |
| 30 | 60 | 6 | 5^{d} | — | 3,892 (1,271) | 5,052 (1,641) | 1.3 (0.2) |
| 100 | 200 | 6 | 1^{b} | 16,250 (2,400) | 13,317 (3,358) | — | — |
| 100 | 200 | 6 | 4^{c} | | — | — | — |
| 100 | 200 | 6 | 5^{d} | — | 6,910 (1,923) | 8,827 (2,713) | 1.3 (0.4) |

—, not assessed/not calculated;
N, number of mice;
SD, standard deviation.
NOTE:
Compound A dosed as a PEG400 solution.
^{a}Lower level of quantification (LLOQ) serum = 10.0 ng/ml; LLOQ tumor = 10.4-27.5 ng/g
^{b}Following first dose.
^{c}Seven doses given.
^{d}Eight doses given.

TABLE 8
Mean (SD) serum and tumor levels in male OCI-LY3 tumor-bearing NSG
mice following once-daily oral doses of Compound A

| | | Compound A concentration^{a} | | | | Tumor:serum |
|---|---|---|---|---|---|---|
| | | Serum (ng/ml) | | | Tumor (ng/g) | ratio |
| | | Time post dose (h) | | | | |
| N | Day | 4 | 12 | 24 | 24 | 24 |
| 6 | 1 | 14,967 (1,999) | — | 2,063 (540) | — | — |
| 6 | 4 | 9,850 (700) | 8,683 (343) | 1,380 (325) | 2,463 (625) | 1.9 (1.0) |
| 6 | 1 | 18,100 (4,239) | — | 1,967 (712) | — | — |
| 6 | 4 | 12,558 (2,829) | 11,033 (2,792) | 2,802 (1,177) | 4,303 (1,370) | 1.6 (0.4) |

—, not assessed/not calculated;
N, number of mice;
SD, standard deviation.
NOTE:
Compound A dosed as a PEG400 solution.
^{a}Lower level of quantification (LLOQ) serum = 10.0 ng/ml; LLOQ tumor = 10.4-27.5 ng/g.

Serum exposure after 28 days of oral administration of vehicle or Compound A at 1, 3, 10, 30, and 100 mg/kg twice daily or 60 mg/kg once daily was evaluated. Serum samples were collected at 1, 2, 4, 8, and 12 hours post dose (for twice-daily-treated animals) or 24 hours (for once-daily-treated animals). Serum exposures results are shown in Table 9 below.

TABLE 9
Mean (SD) serum concentration in male OCI-LY3 tumor-bearing NSG
Mice following 28 days of once- or twice-daily oral doses of Compound A

| | Total | | | | | | | | |
|---|---|---|---|---|---|---|---|---|---|
| | daily | | Serum concentration (ng/ml | | | | | | |
| Dose | dose | | Time post last dose (h) | | | | | | |
| (mg/kg) | (mg/kg) | N | 0.25 | 1 | 2 | 4 | 8 | 12 | 24 |
| 1 BID^{a} | 2 | 3 | 323 | 550 | 449 | 526 | 721 | 307 | — |
| | | | (147) | (123) | (87) | (176) | (321) | (85)^{c} | — |
| 3 BIDª | 6 | 3 | 1,019 | 1,935^{b} | 1,560 | 1,147 | 2,675^{b} | 1,025 | — |
| | | | (151) | | (399) | (200) | | (115) | — |
| 10 BID^{a} | 20 | 3 | 4,047 | 5,080 | 4,603 | 6,247 | 5,497 | 4,168 | — |
| | | | (1,290) | (943) | (436) | (3,449) | (2,307) | (461)^{c} | — |
| 30 BID^{a} | 60 | 3 | 5,967 | 11,400 | 12,283 | 10,367 | 14,700 | 9,000 | — |
| | | | (813) | (721) | (3,029) | (1,595) | (954) | (737) | — |
| 60 QD | 60 | 3 | 6,993 | 9,267 | 15,967 | 11,400 | 10,633 | — | 2,507 |
| | | | (3,281) | (3,800) | (2,442) | (3,360) | (3,907) | | (1,404) |
| 100 BID^{a} | 200 | 3 | 16,500 | 21,233 | 21,633 | 12,833 | 20,500 | 10,338 | — |
| | | | (6,974) | (5,108) | (3,564) | (3.564) | (6,151) | (2,319)^{c} | |

—, not assessed/not calculated;
BID, twice-daily;
QD, once daily;
N, number of mice;
SD, standard deviation.
NOTE:
lower level of quantification (LLOQ) serum = 1 ng/mL for 1, 3, and 10 mg/kg twice daily;
LLOQ = 5 ng/mL for 30 and 100 mg/kg twice daily and 60 mg/kg once daily.
^{a}12-hour twice daily.
^{b}N = 2
^{c}N = 4

### DISCUSSION

The goal of these studies was to assess the pharmacodynamic and antitumor activity of orally administered Compound A, an allosteric inhibitor of MALT1 protease, in CD79b- and a CARD11-mutant ABC-DLBCL xenograft models with constitutive activation of NF-κB signaling. Compound A was assessed at dose levels from 1 to 100 mg/kg (BID or QD) in established OCI-LY3 and OCI-LY10 xenograft models in NSG mice and was well tolerated.

In the OCI-LY3 tumor model, a single dose of 30 and 100 mg/kg of Compound A completely inhibited or reduced serum IL-10 at 12 hours post dose, while an intermediate inhibition or reduction was observed with 10 mg/kg. The inhibition or reduction of IL-10 in serum was lost at 24 hours post dose, correlating with declining Compound A serum exposures in mice from 12 to 24 hours. Compound A also inhibited the ability of MALT1 to cleave BCL 10 substrate in tumors, with maximal increased fraction of uncleaved BCL 10 observed at dose levels ≥10 mg/kg.

In the OCI-LY10 tumor model, single doses of 30 and 100 mg/kg of Compound A completely inhibited or reduced serum IL-10 at 12 hours post dose, while strong inhibition or reduction was observed with 10 mg/kg and an intermediate inhibition was observed with 3 mg/kg.

In the established OCI-LY3 DLBCL model, Compound A induced statistically significant antitumor efficacy at 10, 30, and 100 mg/kg dose levels given twice daily, with 53%, 72%, and 72% TGI observed, respectively, as compared with vehicle control-treated mice. These data were comparable to previous efficacy studies in the OCI-LY3 model, where efficacy with 10 mg/kg BID of Compound A elicited 51-76% TGI, 30 mg/kg BID 60-86% TGI, and 100 mg/kg 62-89% TGI. Overall, in 3 independent OCI-LY3 antitumor efficacy studies, a dose of 10 mg/kg BID of Compound A produced a mean of 60% TGI and is therefore considered the minimum efficacious dose.

In the established OCI-LY10 DLBCL model, statistically significant antitumor activity was elicited with Compound A treatment, with 41%, 54%, and 62% TGI observed, respectively with 10, 30, and 100 mg/kg of Compound A as compared to vehicle treated control animals.

When exposures are dose-proportional, dose fractionation can be used to assess the PK parameters driving antitumor efficacy. Compound A was tested at 60 mg/kg QD in the OCI-LY3 model, where 57% TGI was achieved vs. 72% TGI for 30 mg/kg BID.

### Example 11: Immune Function of MALT 1—Effect of T_{reg}/T_{eff} Ratio Post Stimulation

The role of MALT1 in the immunologic activity of T cells was investigated by looking at the proportions of T_{reg} and effector T cells (T_{eff}). These cells are in a dynamic balance and their ratio correlates with the effectiveness of protective immunity. An accumulation of T_{regs}, resulting in a higher T_{reg}/T_{eff} ratio within tumor tissue, is associated with worse prognosis in cancer. Whiteside T L. What are regulatory T cells (T_{reg}) regulating in cancer and why? Semin Cancer Biol. 2012, 22, 327-34. In vitro, activation of T cells through TCR stimulation increases the T_{reg}/T_{eff} ratio by increasing the T_{reg} population, as defined by CD4⁺CD25^{hi}FOXP3^{hi}.

To assess the effect of Compound A on the T_{reg}/Ter ratio, primary T cells freshly isolated through negative selection from three normal healthy volunteer (NHV) donors, were activated through CD3/28 TCR stimulation. After 24 hours of pre-stimulation, the compound was added for 72 hours. Alternatively, the compound was added together with the CD3/28 stimulus for 96 hours. Cells were stained for flow cytometric analysis (BD Facs Verse). After gating on the living singlet population, the proportions of CD8⁺ T_{eff} and CD4⁺CD25^{hi}FOXP3^{hi} T_{reg} subpopulations were analysed.

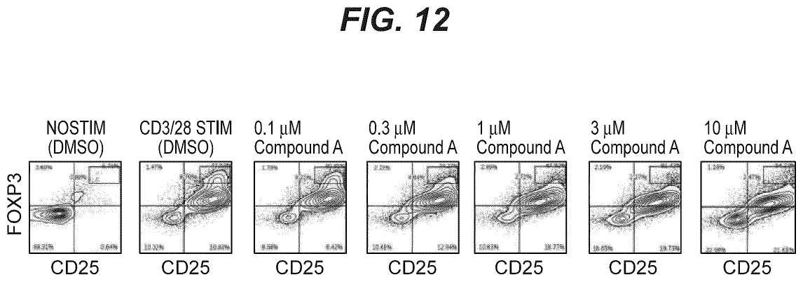

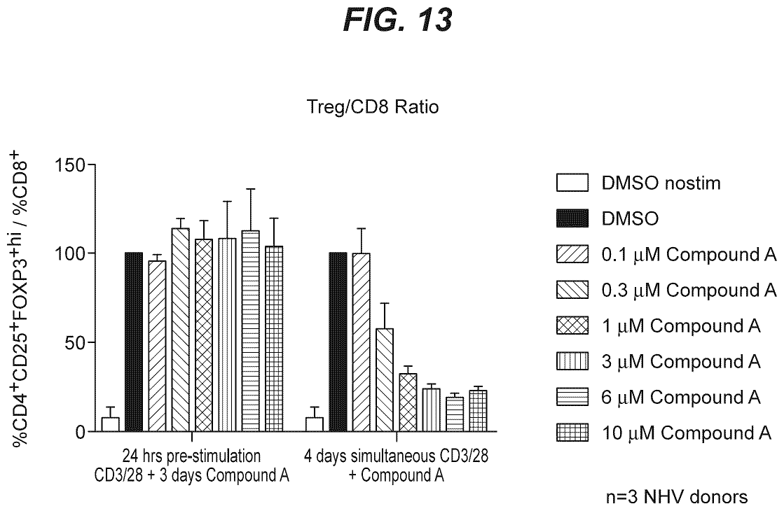

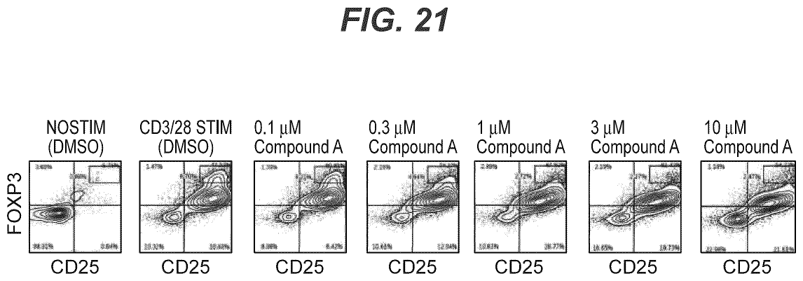

Adding Compound A twenty-four (24) hours after CD3/28 stimulation did not influence the T_{reg} and Ter populations (see FIG. 12, FIG. 21, and FIG. 13). However, if the T cells were stimulated in the presence of Compound A, an increase in the T_{reg}/T_{eff} ratio was prevented in a dose-dependent manner.

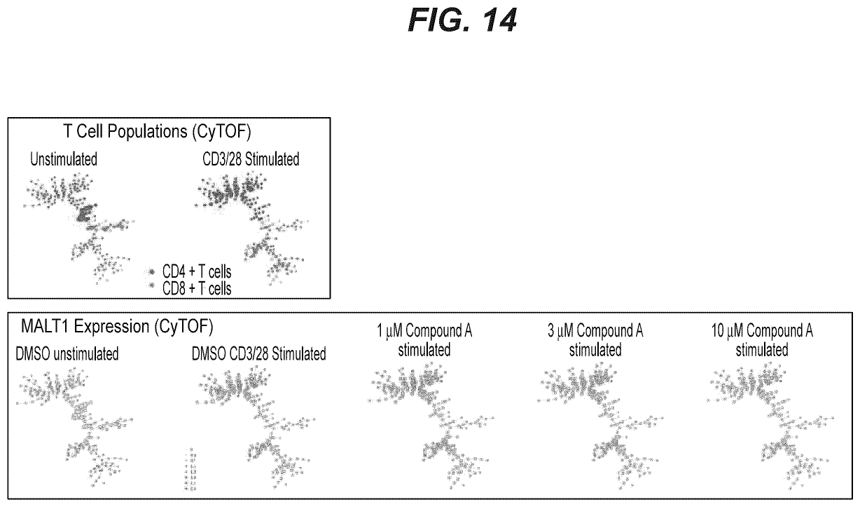

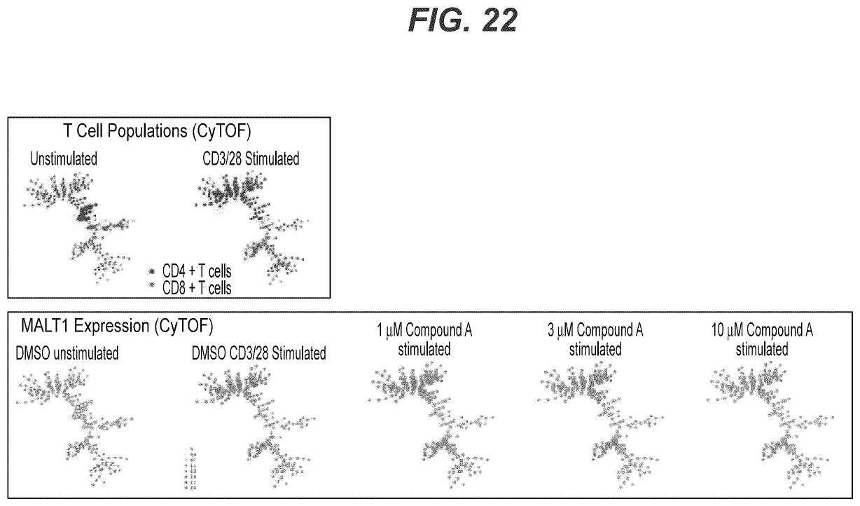

Similarly, T cells derived from four different healthy donors stimulated with CD3/28 for 4 days in the presence of Compound A were analysed using cytometry by time of flight (CyTOF). After fixation and barcoding, samples were pooled per donor and stained using a panel of 32 metal-labelled antibodies for identification of phenotype and function of immune cell populations and acquired on a CyTOF® C5 system. After debarcoding and normalization, cell populations were either manually gated or clustered by spanning-tree progression of density-normalized events (SPADE) analysis using Cytobank® software. Notably, CD3/28 stimulation resulted in an increase in the percentage of MALT1-expressing cells in different populations as well as increased MALT1 protein expression in most populations (see FIG. 14, FIG. 22).

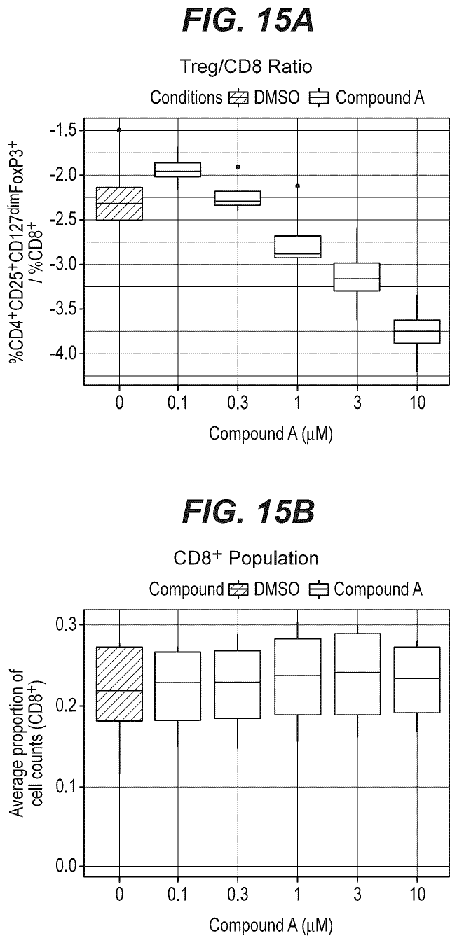

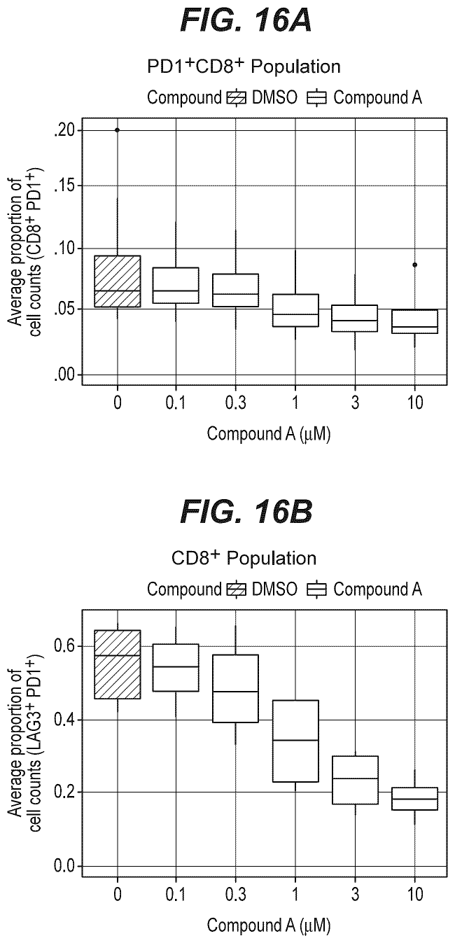

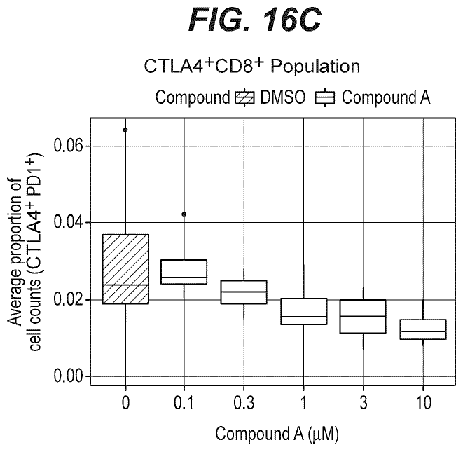

Modelling the manually gated cell populations revealed that treatment with Compound A dose-dependently inhibited the generation of CD4⁺CD25⁺CD1271^{low}FoxP3^{hi} cells following CD3/28 stimulation, while no strong effect on the number of CD8⁺ T cells was observed (FIG. 15A and FIG. 15B). These observations are consistent with the fluorescence-activated cell sorting (FACS) analysis data. Activation of CD8+ T cells was reduced upon treatment with Compound A, in particular, the percentage of double positive CD69⁺CD25⁺ cells following CD3/28 stimulation was decreased while double negative CD69⁻CD25⁻ cells were increased (data not shown). Furthermore, the expression of exhaustion markers, such as PD-1, cytotoxic T-lymphocyte-associated protein 4 (CTLA4), or lymphocyte-activation gene 3 (LAG3), was decreased on CD8⁺ T cells in the presence of Compound A (FIGS. 16A-C). Similar dose-related changes in LAG3 and PD-1 expression were seen in CD4⁺ T cells, whereas CTLA4 expression was not reduced (data not shown). No decrease in the expression of T-cell immunoglobulin and mucin-domain containing-3 (TIM3) was observed in both CD4⁺ and CD8⁺ T cells.

To visualize trends at the single-cell level, Radviz was used. Radviz is a method whereby cells are projected in two dimensions in a manner that preserves their original dimensions and enables rapid interpretation of changes within a population. Treatment effects on specific subsets of cells were visualized using relevant channels representing different activation and functional markers. Radviz shifts were used to guide manual gating and downstream statistical analysis.

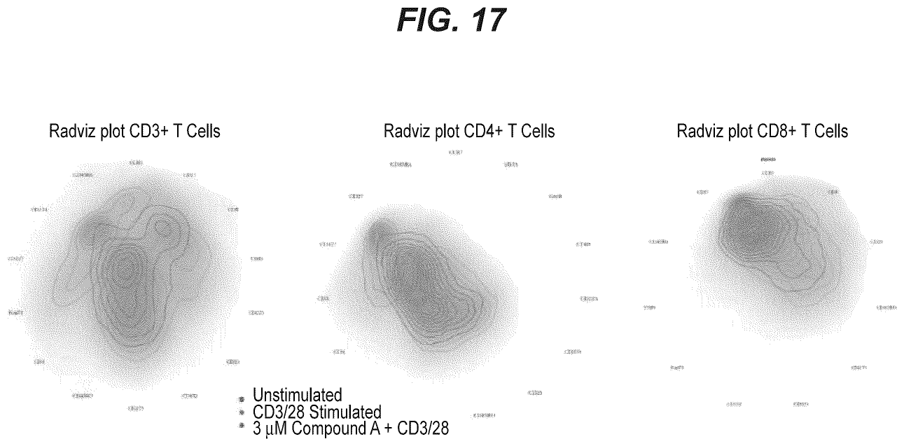

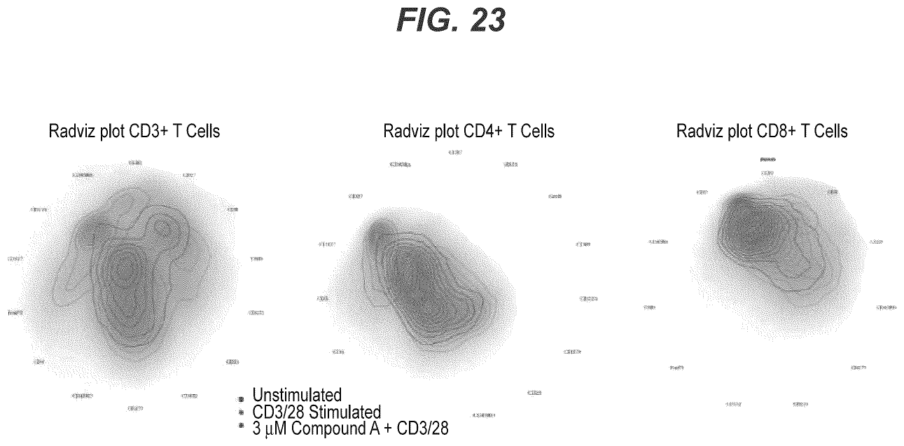

Radviz plots show that T cells altered the expression of multiple markers post CD3/28 stimulation and that treatment with Compound A partially reverted the expression of these markers to the unstimulated condition (FIG. 17, FIG. 23).

### Example 12: Single Dose Pharmacokinetics In Vivo in Mice, Rats, Monkeys, and Dogs

### Intravenous Administration

Single intravenous (IV) dose PK was conducted in mice (two strains), rats, monkeys, and dogs at a dose of 1 mg/kg in 70% PEG400 (PEG400/water 70:30) by bolus injection. The time course for plasma sample was optimized to fully characterize the disposition profile in each species. PK analysis was by non-compartmental method.

The derived PK parameters are shown in Table 10. Following bolus administration, the drug was slowly cleared from all species. The systemic clearance (CL) was much less than liver blood flow (LBF). The fractions of LBF the CL values represented were 1.4, 1.5, 1.7, and 3.0% of the LBF values of 90, 55.2, 43.6, and 30.9 mL/min/kg for mouse, rat, monkey, and dog, respectively. The apparent volume of distribution at steady state (Vass) was similar across all species (˜1 L/kg) and is approximately equivalent to total body water of the animals. The t_{1/2} ranged from 5.28 hours in NSG mice to 16.9 hours in dogs (beagles).

TABLE 10
Mean (SD) plasma PK parameters of Compound A following a single IV
administration in 70% PEG400 solution.

| Species, | N/ | Dose | C₀ | t_{1/2} | V_{dss} | CL | AUC_{inf} |
|---|---|---|---|---|---|---|---|
| strain | sex | (mg/kg) | (ng/mL) | (h) | (L/kg) | (mL/min/kg) | (ng · h/mL) |
| Mouse, CD-1 | 3M | 1 | 940 | — | 1.402 | 1.3 | 12,216 |
| | | | (61) | | (0.175) | (0.2) | (1,763) |
| Mouse, NSG | 3M | 1 | 1,130 | 5.28 | 1.23 | 3.04 | 5,490 |
| | | | (60.1) | (0.087) | (0.053) | (0.116) | (206) |
| Rat, S-D | 3M | 1 | 2,202 | 10.0 | 0.860 | 2.15 | 7,946 |
| | | | (430) | (4.1) | (0.095) | (0.40) | (1,550) |
| Rat, S-D | 6Mª | 1^{b} | 1,450 | 5.33 | 0.622 | 1.70 | 9,780 |
| | | | (120) | | | | |
| Dog, beagle | 3M | 1 | 2,070 | 16.9 | 1.01 | 0.772 | 23,100 |
| | | | (153) | (1.15) | (0.07) | (0.258) | (6,910) |
| Monkey, | 3M | 1 | 1,840 | 11.9 | 0.748 | 1.12 | 16,400 |
| cynomolgus | | | (202) | (3.14) | (0.117) | (0.404) | (6,560) |

—, not assessable/not done;
N, number of animals;
C₀, plasma concentration at time = 0;
AUC_{inf}, area under the plasma concentration-time curve from time = 0 to infinity with extrapolation of the terminal phase;
S-D, Sprague-Dawley;
M, male.
ªN = 3 for first four time points; N = 6 for last (24 hours) time point.
^{b}Dosed as the -ZAF salt; all others -AAA.

### Oral Administration

Following an oral dose of 5 mg/kg administered as a solution in PEG400, the bioavailability was determined in mice, rats, dogs, and monkeys. The bioavailability was >50% in all species and ranged from 51.2% in rats to 100% in dogs. (Data for mice, rats, and monkeys are not shown.)

### Dogs

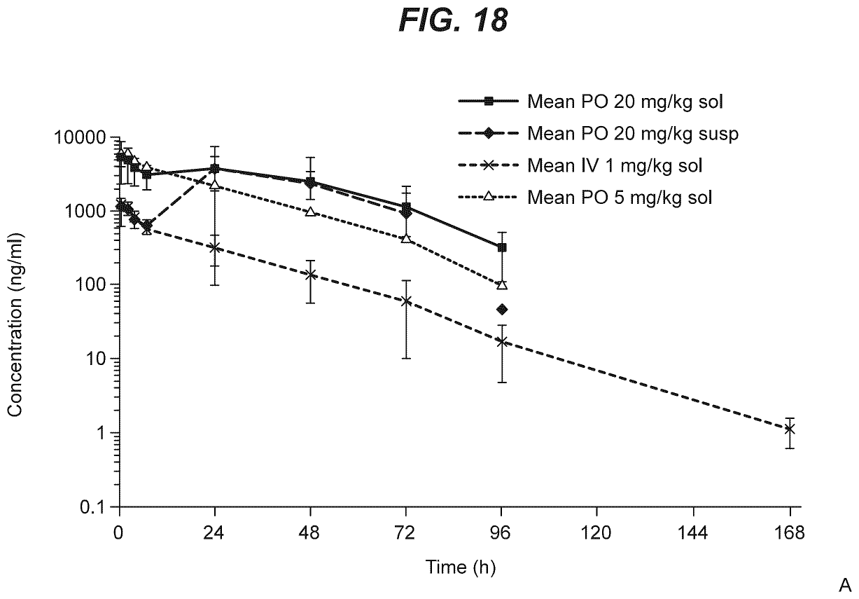

In dogs, presumed precipitation of Compound A from the dosing solution resulted in unusual concentration versus time PK profiles (FIG. 18). This double peak with a second Cₘₐₓ at ˜24 hours leads to nonlinearity in the dose escalation from 5 to 20 mg/kg when Compound A was administered as a solution in PEG400 (Table 11). Also shown in FIG. 18 is the profile following administration of Compound A (as a suspension at 20 mg/kg in 0.5% HPMC). With the suspension formulation the double-peak phenomenon is even more pronounced.

As observed in rats, dose escalation in dogs resulting in proportional or dose-related exposure with the PEG400 solution was unsuccessful in identifying a vehicle suitable for toxicology studies. In Study 7, administration of Compound A at doses of 100, 280, and 560 mg/kg did not increase Cₘₐₓ or AUC greater than approximately 25%. As with rats, a solution formulation of Compound A including the precipitation inhibitor PVP VA in PEG400 resulted in greater exposures and a dose-related but less than proportional increase (approximately 5-fold) in Cₘₐₓ and AUC from 22.5 to 280 mg/kg (Table 11) in dogs. At 480 mg/kg, exposure did not increase relative to the 280 mg/kg dose. These doses were greater than the maximum tolerated dose (MTD) upon 14 days of dosing in dogs.

TABLE 11
Mean (SD) plasma PK parameters of Compound A following a single
oral administration in fasted dogs

| Species, strain | N/ | Dose | Cₘₐₓ | Tₘₐₓ | t_{1/2} | AUCₗₐₛₜ | AUC_{inf} | % |
|---|---|---|---|---|---|---|---|---|
| (study) | sex | (mg/kg) | (ng/ml) | (h) | (h) | (ng · h/mL) | (ng · h/mL) | F |
| Dog, beagle | 3M | 5^{d} | 6,150 | 2 | 17.3 | 143,000^{j} | 146,000 | >100p |
| (Study 1)ª | | | | | | | | |
| Dog, beagle | 3M | 20^{e} | 4,010 | 16.7 | 17.8 | 153,000 | 15,600^{m} | 3.2- |
| (Study 2)^{b} | | | (3,370) | (12.7) | | (160,000)^{k} | | |
| Dog, beagle | 3M | 20^{d} | 5,770 | 8.33 | 18.6 | 215,000 | 224,000 | 33-72 |
| (Study 3) | | | (2,960) | (13.6) | (6.2) | (91,800)^{l} | (96,300) | |
| Dog, beagle | 3M | 5^{e} | 2,030 | 9 | 21.8 | 43,700 | 30,700 | 14-66 |
| (Study 4) | | | (836) | (13) | | (30,500)^{m} | | |
| Dog, beagle | 3M | 20^{e} | 4,080 | 16.7 | 14.3 | 160,000 | 31,800 | 7-89 |
| (Study 4) | | | (5,100) | (12.7) | | (219,000)^{m} | | |
| Dog, beagle | 3M | 5^{f} | 1,450 | 9.33 | 11.5 | 43,700 | 26,400 | 12-69 |
| (Study 5)^{c} | | | (627) | (12.7) | | (33,200)^{m} | | |
| Dog, beagle | 3M | 20^{f} | 3,510 | 9 | 16.6 | 111,000 | 87,000 | 6.7 |
| (Study 5)^{c} | | | (1,850) | (13) | | (72,300)^{m} | | |
| Dog, beagle | 3M | 22.5^{g,h} | 21,000 | 1.25 | 14.1 | 741,000^{l} | 757,000 | >100 |
| (Study 6) | | | | | | | | |
| Dog, beagle | 2F | 100^{d} | 24,300 | 15.5 | — | 1,250,000^{l} | 1,360,000 | — |
| (Study 7) | | | | (7-24) | | | | |
| Dog, beagle | 2F | 280^{d} | 30,600 | 24.0 | — | 1,510,000^{l} | 1,680,000 | — |
| (Study 7) | | | | (24-24)ⁱ | | | | |
| Dog, beagle | 2F | 560^{d} | 31,800 | 24.0 | — | 2,010,000^{l} | 1,760,000ⁿ | — |
| (Study 7) | | | | (24-24)ⁱ | | | | |
| Dog, beagle | 2F | 280^{g} | 106,000 | 24.0 | — | 1,970,000^{l} | —^{o} | — |
| (Study 7) | | | | (24-24)ⁱ | | | | |
| Dog, beagle | 2F | 480^{g} | 76,600 | 27.5 | — | 1,510,000^{l} | | — |
| (Study 7) | | | | (7-48)ⁱ | | | | |

—, not assessable/not done;
N, number of animals;
Tₘₐₓ, time corresponding to the maximum observed plasma concentration;
AUCₗₐₛₜ, AUC from time 0 to the time corresponding to the last quantifiable concentration;
AUC_{inf}, AUC from time = 0 to infinity with extrapolation of the terminal phase;
% F, oral bioavailability expressed as a percent;
F, female;
M, male.
^{a}Dosed 1 mg/kg IV then 5 mg/kg orally after a 1-week washout in a cross-over design. Plasma concentrations were observed in two of the dogs in pre-dose samples of the oral levels were equal to IV 168 hours post dose sample and were used for the PK calculations. Additionally, one dog vomited a large amount between 0.5- and 1-hour post dose, therefore all its data were excluded from parameter calculations (N = 2).
^{b}Dogs dosed in a crossover manner with a 1-week washout between a 5 mg/kg and a 20 mg/kg oral dose, both formulated as a 0.5% HPMC suspension. All 5 mg/kg concentrations were below limit of quantitation (BQL) and therefore only the 20 mg/kg data are reported.
^{c}-ZAF salt dosed as a nanosuspension. In all other studies -AAA salt dosed.
^{d}100% PEG400 solution.
^{e}0.5% HPMC suspension.
^{f}HPMC:sodium dodecyl sulfate (DOSS) suspension.
^{g}PEG400/PVP VA 90:10 solution.
^{h}Formulation ordered at the wrong concentration. Final dose 22.5 mg/kg, instead of 20 mg/kg.
ⁱMedian (range).
^{j}Tₗₐₛₜ = 48 hours.
^{k}Tₗₐₛₜ = 72 hours.
^{l}Tₗₐₛₜ = 96 hours.
^{m}N = 1 animal with low exposure. AUC_{inf} for the other two animals with much higher exposure could not be accurately calculated and therefore are not reported.
ⁿN = 1.
^{o}N = 0.
pCalculated using the IV data from Study 1.

To determine a potential solid oral dosage form for possible use in Phase 1 clinical studies, Compound A was formulated as three different 20 mg capsule blends each containing 10 mg of drug substance (DS) and 10 mg sodium laurel sulfate (SLS), i.e., Form 1, Form 3, and micronized Form 3, and administered to fasted male beagle dogs (N=3/group). Following a single dose, plasma levels were determined up to 24 hours post dose. In that interval, quantifiable concentrations were evident with no real terminal phase, thus limited PK parameters could be calculated.

Peak absorption was observed for all formulations at approximately two hours post dose, except for one dog which received Form 1 where the plasma concentration at 24 hours post dose was 5% greater than at two hours post dose (Table 12). Inter-animal variability was evident resulting in overlap of individual Cₘₐₓ and AUC values. No significant difference between the formulations was evident although the highest exposure was seen in Form 3 and the lowest in Form 1.

TABLE 12
Mean (SD) plasma PK parameters following a single dose
of three 20 mg capsule formulations of Compound A.

| | Form 1 | | Form 3 | | Micronized Form 3 | |
|---|---|---|---|---|---|---|
| Cₘₐₓ (ng/mL) | 396 | (204) | 509 | (162) | 643 | (188) |
| Tₘₐₓ (h) | 9.33 | (12.7) | 2 | | 1.33 | (0.577) |
| AUCₗₐₛₜ | 4,490 | (1,060) | 6,630 | (2,080) | 7,700 | (2,220) |
| (ng · h/mL) | | | | | | |

| | | | |
|---|---|---|---|
| Tₗₐₛₜ (h) | 24 | 24 | 24 |

AUCₗₐₛₜ, area under the plasma concentration-time curve from time 0 to the last quantifiable plasma concentration; SD, standard deviation; Tₗₐₛₜ, time correspondent to the last quantifiable plasma concentration; Tₘₐₓ, time correspondent to the maximum observed plasma concentration All capsules contained a blend of 10 mg drug substance and 10 mg SLS.

### Example 13: Repeated Dose Pharmacokinetics In Vivo in Rats and Dogs

Repeated-dose PK (toxicokinetics) were obtained as part of the repeated oral dose toleration toxicity studies in rats and dogs. Compound A was administered as oral solution formulations in PEG400/PVP VA 90:10.

### Rats

Compound A was administered orally to male and female rats (N=5/sex/group) at doses of 30, 200, and 1,000 mg/kg/day for 14 days, based on the lack of findings seen in the single-dose phase. The daily exposure (Cₘₐₓ and area under the plasma concentration-time curve from time 0 to 24 hours post dose (AUC₀₋₂₄ₕ)) increased from 30 to 200 mg/kg, but less than proportionally with dose (Table 13). There was no further increase in the daily exposure parameters at 1,000 mg/kg relative to those from 200 mg/kg/day. There was no effect of gender on exposure and there was no increase in daily exposure parameters on Day 14 relative to those observed after the first dose.

TABLE 13
Mean toxicokinetic parameters of Compound A, administered
as a PEG400/PVP VA 90:10 solution, in 14-day oral toxicity
studies in Sprague-Dawley rats^{a}

| | | Dose | Cₘₐₓ | Tₘₐₓ^{b} | AUC_{0-24 h} |
|---|---|---|---|---|---|
| N/sex | Day | (mg/kg/day) | (ng/mL) | (h) | (ng · h/mL) |
| 5M | 0 | 30 | 24,400 | 2-4 | 247,000 |
| 5F | 0 | 30 | 27,100 | 1-7 | 366,000 |
| 5M | 0 | 200 | 96,700 | 7-9 | 1,410,000 |
| 5F | 0 | 200 | 93,100 | 93,100 | 93,100 |
| 5M | 0 | 1,000 | 90,800 | 2-9 | 1,120,000 |
| 5F | 0 | 1,000 | 83,200 | 2-4 | 1,140,000 |
| 5M | 13 | 30 | 26,700 | 2-2 | 305,000 |
| 5F | 13 | 30 | 33,400 | 2-4 | 451,000 |
| 5M | 13 | 200 | 63,900 | 2-7 | 803,000 |
| 5F | 13 | 200 | 96,100 | 2-7 | 1,300,000 |
| 4M | 13 | 1,000 | 83,200 | 2-4 | 898,000 |
| 5F | 13 | 1,000 | 83,200 | 83,200 | 83,200 |

N, number of animals;
Tₘₐₓ, time corresponding to the maximum observed plasma concentration;
F, female;
M, male;
RD, repeat dose phase of toxicity study
^{a}Day 0 is the first day of dosing
^{b}Range

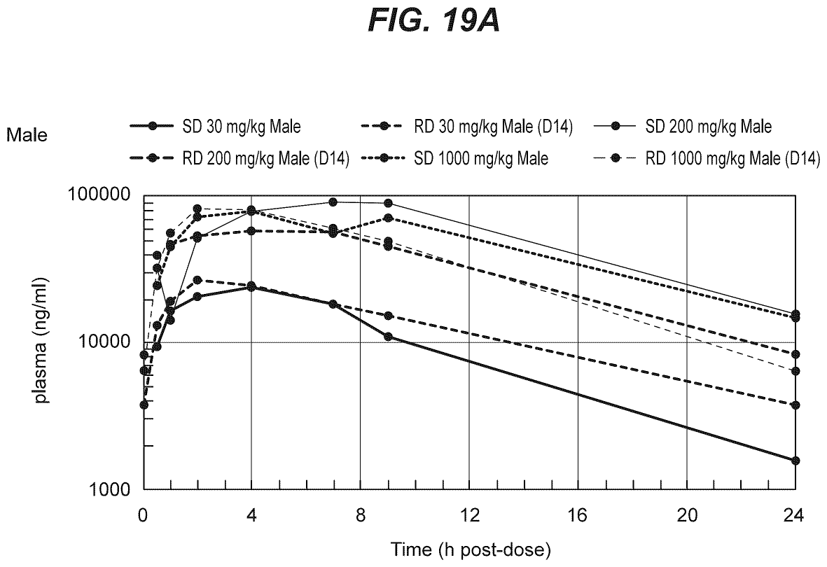

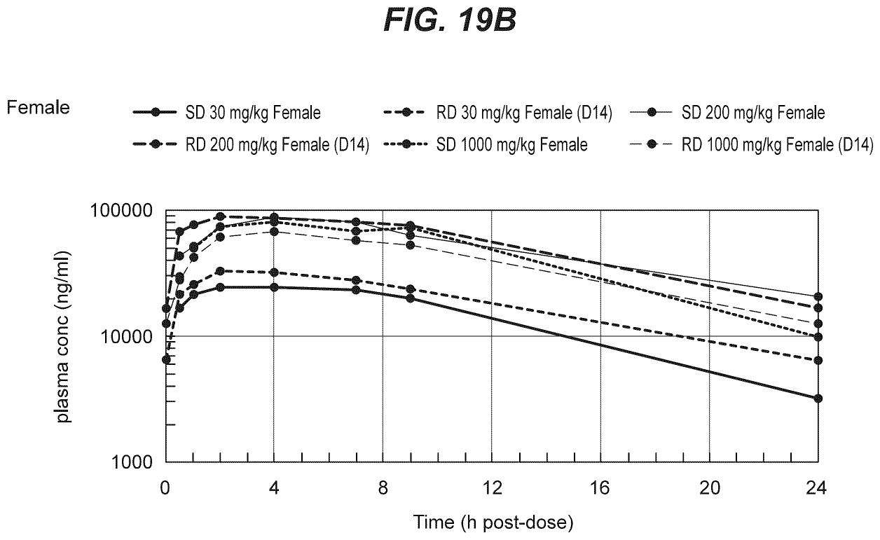

The exposure profiles from the rat toleration studies are shown in FIG. 19A and FIG. 19B, respectively. FIG. 19A shows the exposure profile in male rats. FIG. 19B shows the exposure profile in female rats.

### Dogs

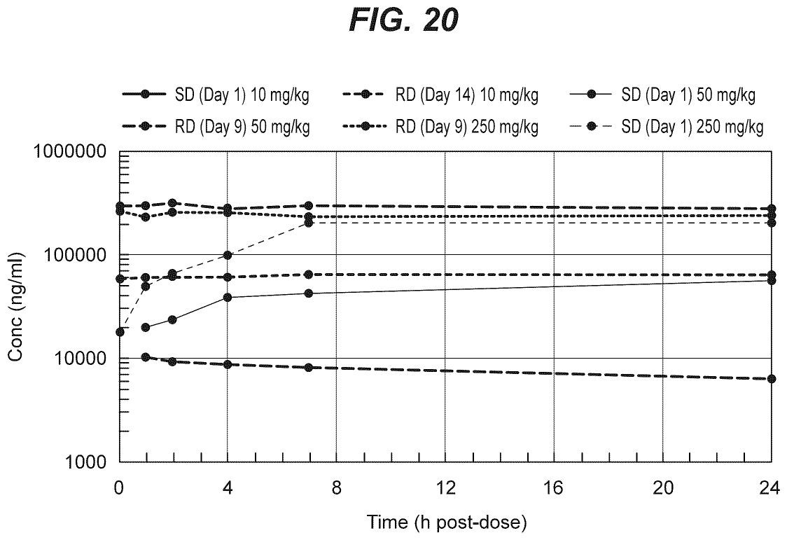

Based on the findings seen in the single-dose phase, Compound A was administered orally to female dogs (beagles) (N=2/group) at doses of 10, 50, and 250 mg/kg/day for 14 days. The daily exposure parameters (Cₘₐₓ and AUC₀₋₂₄ₕ) increased in proportion to dose from 10 to 250 mg/kg/day on Day 1 of dosing, as a 5- and 25-fold increase in dose resulted in a 5.5- and 20-fold increase in Cₘₐₓ and a 5.8- and 23-fold increase in AUC₀₋₂₄ₕ (Table 14). There was an increase in the daily exposure parameters on the last day of plasma sampling (moribundity and mortality resulted in early termination of the 50 and 250 mg/kg dose groups) relative to those measured after the first dose. On Day 14, Cₘₐₓ increased 6.5-fold and AUC₀₋₂₄ₕ increased 8.4-fold at 10 mg/kg/day, and on Day 9, Cₘₐₓ increased 5.6-fold and AUC₀₋₂₄ₕ increased 6.5-fold at 50 mg/kg/day. However, there was no increase in the exposure parameters at 250 mg/kg/day from the first day of dosing until Day 7 when the dose group was terminated. It appears that the increase in exposure seen at 50 mg/kg/day on Day 9 resulted in the highest exposures achieved in the study. The exposure profile from the dog toleration studies is shown in FIG. 20.

TABLE 14
Mean toxicokinetic parameters of Compound A, administered
as a PEG400/PVP VA 90:10 solution, in 14-day oral
toxicity studies in dogs (beagles)^{a}

| | | Dose | Cₘₐₓ | Tₘₐₓ^{b} | AUC_{0-24 h} |
|---|---|---|---|---|---|
| N/sex | Day | (mg/kg/day) | (ng/mL) | (h) | (ng · h/mL) |
| 2F | 1 | 10 | 10,200 | 1-1 | 180,000 |
| 2F | 1 | 50 | 55,900 | 24-24 | 1,050,000 |
| 2F | 7 | 10 | No sampling | | |
| 2F | 7 | 50 | No sampling | | |
| 2F | 7 | 250 | 290,000 | 2-2 | 2,550,000 |
| 2F | 9 | 10 | 41,900 | 2-7 | 936,000 |
| 1F | 9 | 50 | 311,000 | 2-2 | 6,880,000 |
| 1F | 9 | 250 | 263,000 | 0-0 | 5,800,000 |
| 2F | 14 | 10 | 66,500 | 7-24 | 1,520,000 |
| 2F | 14 | 50 | No sampling | | |
| 2F | 14 | 250 | No sampling | | |

N, number of animals;
Tₘₐₓ, time corresponding to the maximum observed plasma concentration;
F, female;
M, male;
RD, repeat dose phase of toxicity study
^{a}Day 1, 7, 9, and 14 recorded as Day 35, 41, 43, and 48 of the study.
^{b}Range

### Example 14: Administration in Humans

Study 67856633LYM1001 is an FIH, open-label study of JNJ-67856633 in subjects with NHL and CLL. The study consists of a dose escalation phase (Part 1), to determine the recommended Phase 2 dose(s) (RP2D[s]), followed by an expansion phase at the RP2D(s) (Part 2). The dose escalation phase is supported using an adaptive dose escalation strategy guided by the modified continual reassessment method (mCRM) based on a Bayesian Logistic Regression Model (BLRM) with escalation with overdose control (EWOC) principle. The mCRM design allows the use of all cumulative dose-limiting toxicities (DLT) data up to the current dose cohort.

Preliminary clinical safety and efficacy data were available for 99 subjects. In Part 1, subjects were enrolled in cohorts of daily oral doses at 50 mg, 100 mg, 200 mg (1×200 mg or 4×50 mg), 300 mg (3×100 mg or 6×50 mg), 400 mg (2×200 mg or 8×50 mg), and 600 mg (3×200 mg), and loading dosing cohorts—400 mg loading dose once daily for 14 days, followed by 300 mg once daily, and 300 mg loading dose twice daily for 7 days followed by 300 mg once daily. In Part 2, subjects received daily oral doses of 300 mg. Because of differences in PK properties between the 50 mg and 200 mg capsules that were observed during the study (i.e, higher and more consistent drug exposure using the 50 mg capsules), subjects treated at doses of 200 mg and 400 mg were assigned to discrete cohorts where the assigned dose was administered using multiples of either 50 mg or 200 mg capsules.

JNJ-67856633 plasma concentration data were available from 85 subjects enrolled in the ongoing FIH study 67856633LYM1001 (74 subjects from Part 1 and 11 subjects from Part 2). In Part 1, PK data were available in subjects receiving doses of 50 mg (1×50 mg), 100 mg (2×50 mg), 200 mg (4×50 mg or 1×200 mg), 300 mg (6×50 mg or 3×100 mg), 400 mg (8×50 mg or 2×200 mg), and 600 mg (3×200 mg). Pharmacokinetic data were also available from the loading dose regimens of 400 mg (8×50 mg) loading dose once daily for 14 days followed by 300 mg (6×50 mg) once daily, and 300 mg (6×50 mg) loading dose twice daily for 7 days followed by 300 mg (6×50 mg) once daily. Mean plasma concentrations following the first dose administration of JNJ-67856633 at Cycle 1 Day 1 and following multiple-dose administration of JNJ-67856633 at Cycle 2 Day 1 are summarized in Table 15.

TABLE 15
Preliminary Mean (% CV) Plasma Pharmacokinetic Parameters of JNJ-
67856633 in Part 1

| | Capsule | | | Cₘₐₓ | Tₘₐₓ^{a} | Cₘᵢₙ | AUC_{τ} | | |
|---|---|---|---|---|---|---|---|---|---|
| Time | strength | N | Cohort | (μg/mL) | (h) | (μg/mL) | (μg · h/mL) | AR_{Cmax} | AR_{AUC} |
| Cycle 1 | 50 mg | 7 | 50 mg | 1.60 | 3.00 | — | 29.1 | — | — |
| Day 1 | | | | (38.6) | (2.00-4.00) | | (37.1) | | |
| | | 7 | 2 × 50 mg | 3.00 | 4.00 | — | 51.4 | — | — |
| | | | | (40.3) | (2.00-4.00) | | (41.2) | | |
| | | 5 | 4 × 50 mg | 6.75^{b} | 4.00^{b} | — | 118^{b} | — | — |
| | | | | (32.0) | (2.00-24.0) | | (32.3) | | |
| | | 10 | 6 × 50 mg | 8.81 | 4.00 | — | 152^{c} | — | — |
| | | | | (43.1) | (1.00-8.00) | | (38.0) | | |
| | | 4 | 8 × 50 mg | 7.56 | 3.00 | — | 131 | — | — |
| | | | | (29.9) | (1.00-24.0) | | (48.4) | | |
| | | 7 | 8 × 50 mg > | 10.3^{d} | 3.00 | | 186^{d} | | |
| | | | 6 × 50 mg | (35.6) | (2.00-24.0) | | (31.0) | | |
| | | 3 | 6 × 50 mg | 4.31 | 6.00 | | — | | |
| | | | BID > QD | (44.6) | (6.00-8.00) | | | | |
| | 200 mg | 5 | 200 mg | 4.06 | 4.00 | — | 61.9 | — | — |
| | | | | (30.3) | (2.00-24.0) | | (31.5) | | |
| | | 6 | 2 × 200 mg | 4.19 | 5.00 | — | 68.0 | — | — |
| | | | | (51.6) | (2.00-24.0) | | (44.4) | | |
| | | 6 | 2 × 200 mg | 6.83 | 4.50 | — | 112 | — | — |
| | | | food adlib | (30.1) | (2.00-8.00) | | (21.9) | | |
| | | 2 | 3 × 200 mg | 7.79, | 3.00, 3.00 | — | 123, 76.6 | — | — |
| | | | | 4.50 | | | | | |
| Cycle 2 | 50 mg | 3 | 50 mg | 8.74 | 2.00 | 6.85 | 180 | 7.30 | 8.37 |
| Day 1 | | | | (3.80) | (0.00-3.00) | (8.00) | (10.0) | (4.70) | (21.4) |
| | | 5 | 2 × 50 mg | 19.9 | 2.00 | 15.6 | 366 | 6.56 | 7.80 |
| | | | | (40.6) | (0.00-3.00) | (58.9) | (34.9) | (27.6) | (23.3) |
| | | 5 | 4 × 50 mg | 44.8^{e} | 2.00^{e} | 30.4^{e} | 882^{e} | 7.32 | 8.10 |
| | | | | (16.6) | (1.00-4.00) | (10.7) | (13.2) | (40.4) | (31.8) |
| | | 7 | 6 × 50 mg | 55.2 | 4.00 | 39.5 | 1144 | 7.32 | 8.50 |
| | | | | (15.3) | (2.00-24.0) | (15.6) | (13.1) | (22.0) | (26.1) |
| | | 4 | 8 × 50 mg | 94.1 | 3.50 | 55.6 | 1588 | 11.3^{f} | 12.4^{f} |
| | | | | (42.9) | (2.00-6.00) | (35.8) | (39.6) | (55.9) | (68.3) |
| | | 7 | 8 × 50 mg > | 72.2^{g} | 3.00^{g} (0.50- | 55.8^{g} | 1700^{j} | 7.7^{g} | 9.80^{g, h} |
| | | | 6 × 50 mg | (30.6) | 24.0) | (31.1) | (14.5) | (40.8) | (24.2) |
| | | 2 | 6 × 50 mg | 29.2, | 3.00, 3.00 | 26.1, | 651, 1406 | 10.4^{j} | — |
| | | | BID > QD | 67.0 | | 50.6 | | | |
| | 200 mg | 2 | 200 mg | 25.7, | 0.5, 2.00 | 15.5, | 453^{j} | 4.81, | 5.51ⁱ |
| | | | | 14.5 | | 11.4 | | 3.21 | |
| | | 3 | 2 × 200 mg | 43.2 | 3.00 | 30.7 | 824 | 7.40 | 9.35 |
| | | | | (46.5) | (0.00-3.00) | (39.9) | (38.4) | (14.4) | (9.50) |
| | | 5 | 2 × 200 mg | 72.3 | 8.00 | 33.3 | 1326 | 12.0 | 12.7 |
| | | | food adlib | (24.4) | (0.50-24.0) | (24.8) | (14.0) | (26.0) | (21.1) |
| | | 2 | 3 × 200 mg | 21.2, | 0.00^{j}, 0.50 | 13.5, 71.2 | 458, 1811 | 2.72, 21.9 | 3.73, 23.7 |
| | | | | 98.6 | | | | | |

— = not assessable/not done;
AUC_{τ} = area under the plasma concentration-time curve over the dosing interval (24 hr);
Cₘₐₓ = maximum observed plasma concentration;
Cₘᵢₙ = minimum observed plasma concentration;
CV = coefficient of variation;
N = number of subjects;
PK = pharmacokinetic;
AR_{Cmax} = accumulation ratio of Cₘₐₓ at Cycle 2 Day 1 and Cycle 1 Day 1;
AR_{AUC} = accumulation ratio of AUC_{τ} at Cycle 2 Day 1 and Cycle 1 Day 1;
Tₘₐₓ = time correspondent to the maximum observed plasma concentration.
^{a}Median (range).
bData from Subject 100032 was excluded from descriptive statistics due to unexpected high exposure compared with other subjects in the same cohort. With Subject 100032 data included, the mean Cₘₐₓ is 8.06 μg/mL with CV of 46.4%, the mean AUC is 139 μg · h/mL with CV of 45.3%.
^{c}n = 9.
Data from Subject 100070 was excluded from descriptive statistics due to unexpected high exposure compared with other subjects in the same cohort. With Subject 100070 data included, the mean Cₘₐₓ is 12.8 μg/mL with CV of 61.3%, the mean AUC is 237 μg · h/mL with CV of 65.1%.
Data from Subject 100032 was excluded from descriptive statistics due to unexpected high exposure compared with other subjects in the same cohort. With Subject 100032 data included, the mean Cₘₐₓ is 58.2 μg/mL with CV of 57.4%, the mean AUC is 1140 μg · h/mL with CV of 56.2%, the Cₘᵢₙ is 40.6 μg/ml, with CV of 62.0%.
n = 3.
Data from Subject 100070 was excluded from descriptive statistics due to unexpected high exposure compared with other subjects in the same cohort. With Subject 100070 data included, the mean Cₘₐₓ is 79.7 μg/mL with CV of 36.9%, the mean AUC is 1813 μg · h/mL with CV of 22.5%, the mean Cₘᵢₙ is 58.8 μg/mL with CV of 30.9%.
n = 6.
n = 1.
Predose at Cycle 2 Day 1.

### PK Following the Daily Dose Regimens (No Loading Dose)

Preliminary PK results following the first JNJ-67856633 oral dose at Cycle 1 Day 1 showed that the median time to reach maximum plasma concentration (Tmax) was 3 to 6 hours at doses of 50 to 600 mg (Table 15). At the dose level of 200 mg (4×50 mg or 1×200 mg) or 400 mg (8×50 mg or 2×200 mg) where both 50 mg and 200 mg capsules were assessed at the same total dose, the maximum plasma concentration (Cmax) and area under the curve over the dosing interval (24 hr) (AUCτ) of JNJ-67856633 using 50 mg capsules were higher than that using 200 mg capsules (Table 15). At Cycle 1 Day 1, the mean Cₘₐₓ and mean AUCτ values at 4×50 mg are 1.66- and 1.91-fold, respectively, of those at 1×200 mg. The mean Cₘₐₓ and AUCτ values at 8×50 mg are 1.80- and 1.93-fold, respectively of those at 2×200 mg.

Preliminary multiple-dose PK of JNJ-67856633 after oral daily administration was evaluated at Cycle 2 Day 1. Median Tₘₐₓ was 2 to 4 hours and 3 to 8 hours following the 50 mg capsule and 200 mg capsule, respectively (Table 15). With the limited data, at the dose level of 200 mg (4×50 mg or 1×200 mg) or 400 mg (8×50 mg or 2×200 mg) where both 50 mg and 200 mg capsules were assessed at the same total dose, Cₘₐₓ and AUCτ of JNJ-67856633 using 50 mg capsules were higher than that using 200 mg capsules (Table 15). At Cycle 2 Day 1, the mean Cₘₐₓ and AUCτ values at 8×50 mg are 2.18- and 1.93-fold, respectively of those at 2×200 mg. When given the 50 mg capsule, Cₘₐₓ and AUCτ of JNJ-67856633 appeared to increase in an approximately dose proportional manner as the dose increased from 50 mg to 400 mg (8×50 mg). JNJ-67856633 accumulated upon multiple dosing after both the 50 mg and 200 mg capsules. Taking all the data from the 50 mg capsule, the mean accumulation ratio is 7.6 (based on Cmax) and 8.8 (based on AUCτ) between the first dose at Cycle 1 Day 1 and multiple doses at Cycle 2 Day 1.

### PK Following the Loading Dose Regimens

The PK exposure (Cₘₐₓ and AUCτ) at Cycle 2 Day 1 following 400 mg (8×50 mg) loading dose once daily for 14 days followed by 300 mg (6×50 mg) once daily was slightly higher than the 300 mg (6×50 mg) once daily (Table 15). This may be due to the variability given that the exposure from these subjects in this cohort also showed slightly higher exposure after the first dose of 400 mg (8×50 mg) compared with the subjects dosed in the 400 mg (8×50 mg) once daily cohort. Limited data are available at Cycle 2 Day 1 following 300 mg (6×50 mg) loading dose twice daily for 7 days followed by 300 mg (6×50 mg) once daily as shown in Table 15.

### Preliminary Food Effect Assessment

Preliminary food effect was assessed in 8 subjects at steady state: 2 subjects at 100 mg (2×50 mg), 1 subject at 400 mg (2×200 mg), 1 subject at 200 mg (4×50 mg) and 4 subjects at 300 mg (3×100 mg). In the fasting condition, JNJ-67856633 was administered after overnight fasting for at least 8 hours. In the fed condition, following an overnight fast of at least 8 hours, the subjects were given a high-fat meal 30 minutes before drug intake. In both conditions, no food was allowed for at least 4 hours after study drug intake. Individual plasma concentrations of JNJ-67856633 under fasting and fed conditions using 50 mg or 200 mg capsules are shown in Table 16. Tₘₐₓ was generally longer under fed conditions (range: 0.5 to 24 hours) compared with fasting conditions (range: 0 to 4 hours), suggesting that food may delay the absorption of JNJ-67856633 (Table 16). The steady state exposure (Cmax and AUCτ) under fasting or fed conditions was comparable.

TABLE 16
Plasma Pharmacokinetic Parameters of JNJ-
67856633 Under Fasting and Fed Conditions

| | | Fasted | Fed | Least squares | 90% |
|---|---|---|---|---|---|
| | | condition | condition | mean ratio | Confidence |
| Dose | | (reference) | (test) | (%)^{b} | Interval (%) |
| 2 × 50 mg | | N = 2 | N = 2 | | |
| | Cₘₐₓ (μg/mL) | 15.3, 18.2 | 13.4, 18.4 | — | — |
| | AUC_{τ} (μg · h/mL) | 313,392 | 304, 421 | — | — |
| | Tₘₐₓ (h) | 2.00, 4.00 | 6.00, 8.00 | — | — |
| 4 × 50 mg | | N = 1 | N = 1 | | |
| | Cₘₐₓ (μg/mL) | 47.5 | 48.4 | — | — |
| | AUC_{τ} (μg · h/mL) | 958 | 1013 | — | — |
| | Tₘₐₓ (h) | 1.00 | 24.0 | — | — |
| 2 × 200 mg | | N = 1 | N = 1 | | |
| | Cₘₐₓ (μg/mL) | 61.1 | 64.0 | — | — |
| | AUC_{τ} (μg · h/mL) | 1097 | 1318 | — | — |

While preferred embodiments of the present invention have been shown and described herein, it will be apparent to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention and that embodiments within the scope of these claims and their equivalents be covered thereby.

### Numbered Embodiments of the Inventions

Provided here are illustrative embodiments of the disclosed technology. These embodiments are illustrative only and do not limit the scope of the present disclosure or of the claims attached.

Embodiment 1. A method of treating a disorder or condition that is affected by the inhibition of MALT1 in a subject in need of treatment, comprising administering a therapeutically effective dose ranging from about 50 mg to about 1000 mg of 1-(1-oxo-1,2-dihydroisoquinolin-5-yl)-5-(trifluoromethyl)-N-[2-(trifluoromethyl)pyridin-4-yl]-1H-pyrazole-4-carboxamide (Compound A):

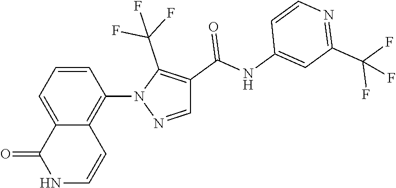 [SMILES: O=C(Nc1ccnc(C(F)(F)F)c1)c1cnn(-c2cccc3c(=O)[nH]ccc23)c1C(F)(F)F]

or a pharmaceutically acceptable salt form thereof to said subject.

Embodiment 2. The method of embodiment 1, wherein the subject is a human.

Embodiment 3. The method of embodiment 1 or 2, wherein the therapeutically effective dose is from about 50 to about 500 mg.

Embodiment 4. The method of embodiment 1 or 2, wherein the therapeutically effective dose is from about 100 to about 400 mg.

Embodiment 5. The method of embodiment 1 or 2, wherein the therapeutically effective dose is from about 150 to about 300 mg.

Embodiment 6. The method of embodiment 1 or 2, wherein the therapeutically effective dose is about 300 mg.

Embodiment 7. The method of embodiment 1 or 2, wherein the therapeutically effective dose is from about 100 to about 150 mg.

Embodiment 8. The method of embodiment 1 or 2, wherein the therapeutically effective dose is from about 150 to about 200 mg.

Embodiment 9. The method of embodiment 1 or 2, wherein the therapeutically effective dose is from about 200 to about 250 mg.

Embodiment 10. The method of embodiment 1 or 2, wherein the therapeutically effective dose is from about 250 to about 300 mg.

Embodiment 11. The method of embodiment 1 or 2, wherein the therapeutically effective dose is from about 300 to 350 mg.

Embodiment 12. The method of embodiment 1 or 2, wherein the therapeutically effective dose is from about 350 to 400 mg.

Embodiment 13. The method of any one of embodiments 1-12, wherein the therapeutically effective dose is divided in half, said half dose being administered twice (two times) a day.

Embodiment 14. The method of any one of embodiments 1-12, wherein the therapeutically effective dose is administered one time a day.

Embodiment 15. The method of any one of embodiments 1-14, wherein the therapeutically effective dose is administered daily on a continuous 28-day cycle.

Embodiment 16. The method of any one of embodiments 1-14, wherein the therapeutically effective dose is administered daily on a continuous 21-day cycle.

Embodiment 17. A method of treating a disorder or condition that is affected by the inhibition of MALT1 in a subject in need of treatment, comprising administering a therapeutic effective dose of 1-(1-oxo-1,2-dihydroisoquinolin-5-yl)-5-(trifluoromethyl)-N-[2-(trifluoromethyl)pyridin-4-yl]-1H-pyrazole-4-carboxamide (Compound A):

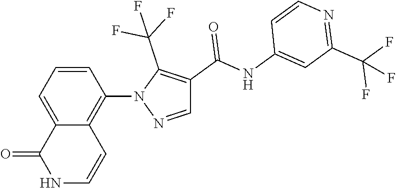 [SMILES: O=C(Nc1ccnc(C(F)(F)F)c1)c1cnn(-c2cccc3c(=O)[nH]ccc23)c1C(F)(F)F]

or a pharmaceutically acceptable salt form thereof to said subject in an amount sufficient to maintain a plasma level of Compound A from about 2 μg/ml to about 120 μg/ml, about 2 μg/ml to about 100 μg/ml, about 2 μg/ml to about 80 μg/ml, about 2 μg/ml to about 60 μg/ml, or about 2 μg/ml to about 20 μg/ml.

Embodiment 18. A method of treating a disorder or condition that is affected by the inhibition of MALT1 in a subject in need of treatment, comprising administering a therapeutic effective dose of 1-(1-oxo-1,2-dihydroisoquinolin-5-yl)-5-(trifluoromethyl)-N-[2-(trifluoromethyl)pyridin-4-yl]-1H-pyrazole-4-carboxamide (Compound A):

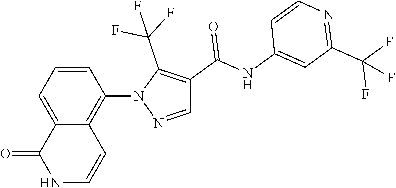 [SMILES: O=C(Nc1ccnc(C(F)(F)F)c1)c1cnn(-c2cccc3c(=O)[nH]ccc23)c1C(F)(F)F]

or a pharmaceutically acceptable salt form thereof to said subject in an amount sufficient to achieve an AUC from about 50 μg·h/ml to about 2500 μg·h/ml, about 50 μg·h/ml to about 2000 μg·h/ml, about 50 μg·h/ml to about 1500 μg·h/ml, about 50 μg·h/ml to about 1000 μg·h/ml, or about 50 μg·h/ml to about 600 μg·h/ml.

Embodiment 19. A method of treating a cancer or an immunological disease in a subject in need of treatment, comprising administering a therapeutic effective dose of 1-(1-oxo-1,2-dihydroisoquinolin-5-yl)-5-(trifluoromethyl)-N-[2-(trifluoromethyl)pyridin-4-yl]-1H-pyrazole-4-carboxamide (Compound A):

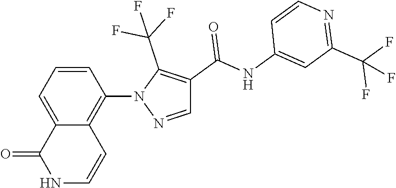 [SMILES: O=C(Nc1ccnc(C(F)(F)F)c1)c1cnn(-c2cccc3c(=O)[nH]ccc23)c1C(F)(F)F]

or a pharmaceutically acceptable salt form thereof to said subject in an amount sufficient to maintain a plasma level of Compound A from about 2 μg/ml to about 120 μg/ml, about 2 μg/ml to about 100 μg/ml, about 2 μg/ml to about 80 μg/ml, about 2 μg/ml to about 60 μg/ml, or about 2 μg/ml to about 20 μg/ml.

Embodiment 20. A method of treating a cancer or an immunological disease in a subject in need of treatment, comprising administering 1-(1-oxo-1,2-dihydroisoquinolin-5-yl)-5-(trifluoromethyl)-N-[2-(trifluoromethyl)pyridin-4-yl]-1H-pyrazole-4-carboxamide (Compound A):

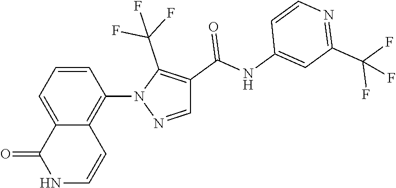 [SMILES: O=C(Nc1ccnc(C(F)(F)F)c1)c1cnn(-c2cccc3c(=O)[nH]ccc23)c1C(F)(F)F]

or a pharmaceutically acceptable salt form thereof to said subject in an amount sufficient to achieve an AUC from about 50 μg·h/ml to about 2500 μg·h/ml, about 50 μg·h/ml to about 2000 μg·h/ml, about 50 μg·h/ml to about 1500 μg·h/ml, about 50 μg·h/ml to about 1000 μg·h/ml, or about 50 μg·h/ml to about 600 μg·h/ml.

Embodiment 21. The method of any one of embodiments 1-20, wherein the disorder or condition is a cancer selected from lymphomas, leukemias, carcinomas, and sarcomas, e.g. non-Hodgkin's lymphoma (NHL (including B-cell NHL)), diffuse large B-cell lymphoma (DLBCL), mantle cell lymphoma (MCL), follicular lymphoma (FL), mucosa-associated lymphoid tissue (MALT) lymphoma, marginal zone lymphoma, T-cell lymphoma, Hodgkin's lymphoma, Burkitt's lymphoma, multiple myeloma, chronic lymphocytic leukemia (CLL), small lymphocytic lymphoma (SLL), Waldenström macroglobulinemia, lymphoblastic T cell leukemia, chronic myelogenous leukemia (CML), hairy-cell leukemia, acute lymphoblastic T cell leukemia, plasmacytoma, immunoblastic large cell leukemia, megakaryoblastic leukemia, acute megakaryocyte leukemia, promyelocytic leukemia, erythroleukemia, brain (gliomas), glioblastomas, breast cancer, colorectal/colon cancer, prostate cancer, lung cancer including non-small-cell, gastric cancer, endometrial cancer, melanoma, pancreatic cancer, liver cancer, kidney cancer, squamous cell carcinoma, ovarian cancer, sarcoma, osteosarcoma, thyroid cancer, bladder cancer, head & neck cancer, testicular cancer, Ewing's sarcoma, rhabdomyosarcoma, medulloblastoma, neuroblastoma, cervical cancer, renal cancer, urothelial cancer, vulval cancer, esophageal cancer, salivary gland cancer, nasopharyngeal cancer, buccal cancer, cancer of the mouth, primary and secondary central nervous system lymphoma, transformed follicular lymphoma, diseases/cancer caused by API2-MALT1 fusion, and GIST (gastrointestinal stromal tumor).

Embodiment 22. The method of any one of embodiments 1-20, wherein the the disorder or condition is an immunological disease selected from autoimmune and inflammatory disorders, e.g. arthritis, rheumatoid arthritis (RA), psoriatic arthritis (PsA), inflammatory bowel disease, gastritis, ankylosing spondylitis, ulcerative colitis, pancreatitis, Crohn's disease, celiac disease, multiple sclerosis, systemic lupus erythematosus, lupus nephritis, rheumatic fever, gout, organ or transplant rejection, chronic allograft rejection, acute or chronic graft-versus-host disease, dermatitis including atopic, dermatomyositis, psoriasis, Behcet's diseases, uveitis, myasthenia gravis, Grave's disease, Hashimoto thyroiditis, Sjoergen's syndrome, blistering disorders, antibody-mediated vasculitis syndromes, immune-complex vasculitides, allergic disorders, asthma, bronchitis, chronic obstructive pulmonary disease (COPD), cystic fibrosis, pneumonia, pulmonary diseases including oedema, embolism, fibrosis, sarcoidosis, hypertension and emphysema, silicosis, respiratory failure, acute respiratory distress syndrome, BENTA disease, berylliosis, and polymyositis.

Embodiment 23. The method of any one of embodiments 1-20, wherein the disorder or condition is selected from non-Hodgkin's lymphoma (NHL), diffuse large B-cell lymphoma (DLBCL), marginal zone lymphoma, mantle cell lymphoma (MCL), follicular lymphoma (FL), transformed follicular lymphoma, chronic lymphocytic leukemia, and Waldenström macroglobulinemia.

Embodiment 24. A method of treating non-Hodgkin's lymphoma (NHL) in a subject comprising administering to the subject a therapeutically effective dose of about 100 mg to about 300 mg of Compound A.

Embodiment 25. A method of treating diffuse large B-cell lymphoma (DLBCL) in a subject comprising administering to the subject a therapeutically effective dose of about 100 mg to about 300 mg of Compound A.

Embodiment 26. A method of treating marginal zone lymphoma (MZL) in a subject comprising administering to the subject a therapeutically effective dose of about 100 mg to about 300 mg of Compound A.

Embodiment 27. A method of treating mantle cell lymphoma (MCL) in a subject comprising administering to the subject a therapeutically effective dose of about 100 mg to about 300 mg of Compound A.

Embodiment 28. A method of treating follicular lymphoma (FL) in a subject comprising administering to the subject a therapeutically effective dose of about 100 mg to about 300 mg of Compound A.

Embodiment 29. A method of treating transformed follicular lymphoma (tFL) in a subject comprising administering to the subject a therapeutically effective dose of about 100 mg to about 300 mg of Compound A.

Embodiment 30. A method of treating chronic lymphocytic leukemia (CLL) in a subject comprising administering to the subject a therapeutically effective dose of about 100 mg to about 300 mg of Compound A.

Embodiment 31. A method of treating Waldenström macroglobulinemia in a subject comprising administering to the subject a therapeutically effective dose of about 100 mg to about 300 mg of Compound A.

Embodiment 32. The method of embodiment 25, wherein the DLBCL is the activated B cell like (ABC) subtype of diffuse large B-cell lymphoma (DLBCL).

Embodiment 33. The method of embodiment 25, wherein the DLBCL is germinal center B cell like (GCB) subtype of diffuse large B-cell lymphoma (DLBCL).

Embodiment 34. The method of embodiment 25, wherein the DLBCL is non-germinal center B cell like (non-GCB) subtype of diffuse large B-cell lymphoma (DLBCL).

Embodiment 35. The method of any one of embodiments 24-34, wherein the method comprises administering about 100 mg to about 300 mg of Compound A once daily for 7 to 21 continuous day cycle, and optionally 3-10 cycles.

Embodiment 36. The method of any one of embodiments 24-34, wherein the method comprises administering about 100 mg to about 300 mg of Compound A twice daily for 7 days followed by administration of about 100 mg to about 300 mg of Compound A once a day for 14 days, and optionally 3-10 cycles.

Embodiment 37. The method of any one of embodiments 24-34, wherein the method comprises administering a therapeutically effective dose 100 mg to about 300 mg of Compound A twice daily for 7 days followed by administration of about 100 mg to about 300 mg of Compound A once a day until remission.

Embodiment 38. The method of any one of embodiments 24-34, wherein said subject has received prior treatment with a Bruton tyrosine kinase inhibitor (BTKi).

Embodiment 39. The method of any one of embodiments 24-34, wherein said subject is relapsed or refractory to prior treatment with a Bruton tyrosine kinase inhibitor (BTKi).

Embodiment 40. The method of any one of embodiments 1-39, wherein Compound A is used as a hydrate or a monohydrate form thereof.

Embodiment 41. The method of any one of embodiments 1-40, wherein said subject is administered a pharmaceutical composition of Compound A or a pharmaceutically acceptable salt form thereof further comprising a pharmaceutically acceptable carrier, a pharmaceutically acceptable excipient, and/or a pharmaceutically acceptable diluent.

Embodiment 42. 1-(1-oxo-1,2-dihydroisoquinolin-5-yl)-5-(trifluoromethyl)-N-[2-(trifluoromethyl)pyridin-4-yl]-1H-pyrazole-4-carboxamide (Compound A):

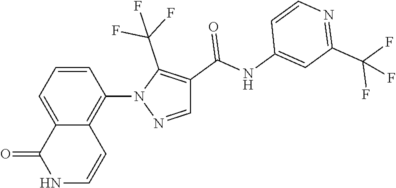 [SMILES: O=C(Nc1ccnc(C(F)(F)F)c1)c1cnn(-c2cccc3c(=O)[nH]ccc23)c1C(F)(F)F]

or a pharmaceutically acceptable salt form thereof for use in treating a disorder or condition that is affected by the inhibition of MALT1, wherein the use comprises a therapeutically effective dose of from about 50 mg to about 1000 mg of Compound A or a pharmaceutically acceptable salt form thereof.

Embodiment 43. A pharmaceutical composition comprising 1-(1-oxo-1,2-dihydroisoquinolin-5-yl)-5-(trifluoromethyl)-N-[2-(trifluoromethyl)pyridin-4-yl]-1H-pyrazole-4-carboxamide (Compound A):

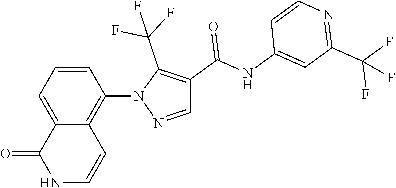 [SMILES: O=C(Nc1ccnc(C(F)(F)F)c1)c1cnn(-c2cccc3c(=O)[nH]ccc23)c1C(F)(F)F]

or a pharmaceutically acceptable salt form thereof for use in treating a disorder or condition that is affected by the inhibition of MALT1, wherein the use comprises a therapeutically effective dose of about 50 mg to about 1000 mg of Compound A or a pharmaceutically acceptable salt form thereof.

Embodiment 44. The Compound A or a pharmaceutically acceptable salt form thereof for use according to embodiment 42 or the pharmaceutical composition for use according to embodiment 43, wherein the use comprises: a therapeutically effective dose of from about 50 to about 1000 mg; a therapeutically effective dose of from about 50 to about 500 mg; a therapeutically effective dose of from about 100 to about 400 mg; a therapeutically effective dose of from about 150 to about 300 mg; a therapeutically effective dose of about 200 mg; a therapeutically effective dose of from about 100 to about 150 mg; a therapeutically effective dose of from about 150 to about 200 mg; a therapeutically effective dose of from about 200 to about 250 mg; a therapeutically effective dose of from about 250 to about 300 mg; a therapeutically effective dose of from about 300 to 350 mg; or a therapeutically effective dose of from about 350 to 400 mg.

Embodiment 45. The Compound A or a pharmaceutically acceptable salt form thereof for use according to embodiments 42 or 44, or the pharmaceutical composition for use according to embodiments 43 or 44, wherein the therapeutically effective dose is divided in half, said half dose being administered twice (two times) a day.

Embodiment 46. The Compound A or a pharmaceutically acceptable salt form thereof for use according to embodiments 42 or 44, or the pharmaceutical composition for use according to embodiments 43 or 44, wherein the therapeutically effective dose is administered one time a day.

Embodiment 47. The Compound A or a pharmaceutically acceptable salt form thereof for use according to embodiments 42 or 44, or the pharmaceutical composition for use according to embodiments 43 or 44, wherein the therapeutically effective dose is administered daily on a continuous 28-day cycle.

Embodiment 48. The Compound A or a pharmaceutically acceptable salt form thereof for use according to embodiments 42 or 44, or the pharmaceutical composition for use according to embodiments 43 or 44, wherein the therapeutically effective dose is administered daily on a continuous 21-day cycle.

Embodiment 49. The Compound A or a pharmaceutically acceptable salt form thereof for use according to embodiments 42 or 44, wherein Compound A is used as ta hydrate or a monohydrate form thereof.

Embodiment 50. The pharmaceutical composition for use according to embodiments 43 or 44, wherein Compound A is used as a hydrate or a monohydrate form thereof.

Embodiment 51. The Compound A or a pharmaceutically acceptable salt form thereof for use or the pharmaceutical composition for use according to any one of embodiments 42-50, wherein said disorder or condition is a cancer selected from lymphomas, leukemias, carcinomas, and sarcomas, e.g. non-Hodgkin's lymphoma (NHL (including B-cell NHL)), diffuse large B-cell lymphoma (DLBCL), mantle cell lymphoma (MCL), follicular lymphoma (FL), mucosa-associated lymphoid tissue (MALT) lymphoma, marginal zone lymphoma, T-cell lymphoma, Hodgkin's lymphoma, Burkitt's lymphoma, multiple myeloma, chronic lymphocytic leukemia (CLL), small lymphocytic lymphoma (SLL), Waldenström macroglobulinemia, lymphoblastic T cell leukemia, chronic myelogenous leukemia (CML), hairy-cell leukemia, acute lymphoblastic T cell leukemia, plasmacytoma, immunoblastic large cell leukemia, megakaryoblastic leukemia, acute megakaryocyte leukemia, promyelocytic leukemia, erythroleukemia, brain (gliomas), glioblastomas, breast cancer, colorectal/colon cancer, prostate cancer, lung cancer including non-small-cell, gastric cancer, endometrial cancer, melanoma, pancreatic cancer, liver cancer, kidney cancer, squamous cell carcinoma, ovarian cancer, sarcoma, osteosarcoma, thyroid cancer, bladder cancer, head & neck cancer, testicular cancer, Ewing's sarcoma, rhabdomyosarcoma, medulloblastoma, neuroblastoma, cervical cancer, renal cancer, urothelial cancer, vulval cancer, esophageal cancer, salivary gland cancer, nasopharyngeal cancer, buccal cancer, cancer of the mouth, primary and secondary central nervous system lymphoma, transformed follicular lymphoma, diseases/cancer caused by API2-MALT1 fusion, and GIST (gastrointestinal stromal tumor).

Embodiment 52. The Compound A or a pharmaceutically acceptable salt form thereof for use or the pharmaceutical composition for use according to any one of embodiments 42-50, wherein said disorder or condition is an immunological disease selected from autoimmune and inflammatory disorders, e.g. arthritis, rheumatoid arthritis (RA), psoriatic arthritis (PsA), inflammatory bowel disease, gastritis, ankylosing spondylitis, ulcerative colitis, pancreatitis, Crohn's disease, celiac disease, multiple sclerosis, systemic lupus erythematosus, lupus nephritis, rheumatic fever, gout, organ or transplant rejection, chronic allograft rejection, acute or chronic graft-versus-host disease, dermatitis including atopic, dermatomyositis, psoriasis, Behcet's diseases, uveitis, myasthenia gravis, Grave's disease, Hashimoto thyroiditis, Sjoergen's syndrome, blistering disorders, antibody-mediated vasculitis syndromes, immune-complex vasculitides, allergic disorders, asthma, bronchitis, chronic obstructive pulmonary disease (COPD), cystic fibrosis, pneumonia, pulmonary diseases including oedema, embolism, fibrosis, sarcoidosis, hypertension and emphysema, silicosis, respiratory failure, acute respiratory distress syndrome, BENTA disease, berylliosis, and polymyositis.

Embodiment 53. Use of 1-(1-oxo-1,2-dihydroisoquinolin-5-yl)-5-(trifluoromethyl)-N-[2-(trifluoromethyl)pyridin-4-yl]-1H-pyrazole-4-carboxamide (Compound A):

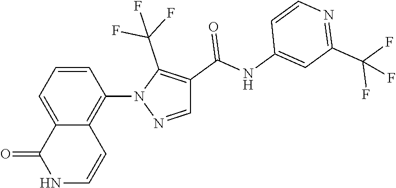 [SMILES: O=C(Nc1ccnc(C(F)(F)F)c1)c1cnn(-c2cccc3c(=O)[nH]ccc23)c1C(F)(F)F]

or a pharmaceutically acceptable salt form thereof for treating a disorder or condition that is affected by the inhibition of MALT1, wherein the use comprises a therapeutically effective dose of from about 50 mg to about 1000 mg of Compound A or a pharmaceutically acceptable salt form thereof.

Embodiment 54. The use of embodiment 53, wherein the use comprises: a therapeutically effective dose of from about 50 to about 1000 mg; a therapeutically effective dose of from about 50 to about 500 mg; a therapeutically effective dose of from about 100 to about 400 mg; a therapeutically effective dose of from about 150 to about 300 mg; a therapeutically effective dose of from about 200 mg; a therapeutically effective dose of from about 100 to about 150 mg; a therapeutically effective dose of from about 150 to about 200 mg; a therapeutically effective dose of from about 200 to about 250 mg; a therapeutically effective dose of from about 250 to about 300 mg; a therapeutically effective dose of from about 300 to 350 mg; or a therapeutically effective dose of from about 350 to 400 mg.

Embodiment 55. The use of embodiments 53 or 54, wherein the therapeutically effective dose is divided in half, said half dose being administered twice (two times) a day.

Embodiment 56. The use of any one of embodiments 53-55, wherein the therapeutically effective dose is: administered one time a day; administered daily on a continuous 28-day cycle; or administered daily on a continuous 21-day cycle.

Embodiment 57. The use of any one of embodiments 53-56, wherein the use comprises: an amount sufficient to maintain a plasma level of Compound A from about 2,300 ng/mL to about 9,300 ng/ml; an amount sufficient to maintain a plasma level of Compound A from about 2,320 ng/mL to about 9,280 ng/ml; an amount sufficient to maintain a plasma level of Compound A from about 3,000 ng/mL to about 9,000 ng/ml; an amount sufficient to maintain a plasma level of Compound A from about 3,500 ng/mL to about 8,500 ng/ml; an amount sufficient to maintain a plasma level of Compound A from about 4,000 ng/ml to about 8,000 ng/ml; an amount sufficient to maintain a plasma level of Compound A from about 4,000 ng/ml to about 6,000 ng/ml; an amount sufficient to maintain a plasma level of Compound A of at least 4,600 ng/mL; an amount sufficient to maintain a plasma level of Compound A from about 4,500 ng/ml to about 4,750 ng/ml; an amount sufficient to maintain a plasma level of Compound A from about 4,640 ng/ml; an amount sufficient to maintain a plasma level of Compound A from about 4,550 to about 4,700 ng/ml; or an amount sufficient to maintain a plasma level of Compound A from about 4,550 to about 4,680 ng/ml.

Embodiment 58. The use of any one of embodiments 53-57, wherein Compound A is used as a hydrate or a monohydrate form thereof.

Embodiment 59. The use of any one of embodiments 53-58, wherein the use comprises a pharmaceutical composition of Compound A or a solvate or pharmaceutically acceptable salt form thereof further comprising a pharmaceutically acceptable carrier, a pharmaceutically acceptable excipient, and/or a pharmaceutically acceptable diluent.

Embodiment 60. The use of any one of embodiments 53-59, wherein said disorder or condition is a cancer selected from lymphomas, leukemias, carcinomas, and sarcomas, e.g. non-Hodgkin's lymphoma (NHL (including B-cell NHL)), diffuse large B-cell lymphoma (DLBCL), mantle cell lymphoma (MCL), follicular lymphoma (FL), mucosa-associated lymphoid tissue (MALT) lymphoma, marginal zone lymphoma, T-cell lymphoma, Hodgkin's lymphoma, Burkitt's lymphoma, multiple myeloma, chronic lymphocytic leukemia (CLL), small lymphocytic lymphoma (SLL), Waldenström macroglobulinemia, lymphoblastic T cell leukemia, chronic myelogenous leukemia (CML), hairy-cell leukemia, acute lymphoblastic T cell leukemia, plasmacytoma, immunoblastic large cell leukemia, megakaryoblastic leukemia, acute megakaryocyte leukemia, promyelocytic leukemia, erythroleukemia, brain (gliomas), glioblastomas, breast cancer, colorectal/colon cancer, prostate cancer, lung cancer including non-small-cell, gastric cancer, endometrial cancer, melanoma, pancreatic cancer, liver cancer, kidney cancer, squamous cell carcinoma, ovarian cancer, sarcoma, osteosarcoma, thyroid cancer, bladder cancer, head & neck cancer, testicular cancer, Ewing's sarcoma, rhabdomyosarcoma, medulloblastoma, neuroblastoma, cervical cancer, renal cancer, urothelial cancer, vulval cancer, esophageal cancer, salivary gland cancer, nasopharyngeal cancer, buccal cancer, cancer of the mouth, primary and secondary central nervous system lymphoma, transformed follicular lymphoma, diseases/cancer caused by API2-MALT1 fusion, and GIST (gastrointestinal stromal tumor).

Embodiment 61. The use of any one of embodiments 53-59, wherein said disorder or condition is an immunological disease selected from autoimmune and inflammatory disorders, e.g. arthritis, rheumatoid arthritis (RA), psoriatic arthritis (PsA), inflammatory bowel disease, gastritis, ankylosing spondylitis, ulcerative colitis, pancreatitis, Crohn's disease, celiac disease, multiple sclerosis, systemic lupus erythematosus, lupus nephritis, rheumatic fever, gout, organ or transplant rejection, chronic allograft rejection, acute or chronic graft-versus-host disease, dermatitis including atopic, dermatomyositis, psoriasis, Behcet's diseases, uveitis, myasthenia gravis, Grave's disease, Hashimoto thyroiditis, Sjoergen's syndrome, blistering disorders, antibody-mediated vasculitis syndromes, immune-complex vasculitides, allergic disorders, asthma, bronchitis, chronic obstructive pulmonary disease (COPD), cystic fibrosis, pneumonia, pulmonary diseases including oedema, embolism, fibrosis, sarcoidosis, hypertension and emphysema, silicosis, respiratory failure, acute respiratory distress syndrome, BENTA disease, berylliosis, and polymyositis.

Embodiment 62. The use of any one of embodiments 53-61, wherein said disorder or condition is relapsed or refractory to prior treatment

Embodiment 63. The use of any one of embodiments 53-61, wherein said disorder or condition is relapsed or refractory to prior treatment with a Bruton tyrosine kinase inhibitor (BTKi).

Embodiment 64. A method of reducing the Tree/T_{eff} ratio in a patient suffering from a disorder or condition that is affected by the inhibition of MALT1 comprising administering a therapeutically effective dose of 1-(1-oxo-1,2-dihydroisoquinolin-5-yl)-5-(trifluoromethyl)-N-[2-(trifluoromethyl)pyridin-4-yl]-1H-pyrazole-4-carboxamide (Compound A):

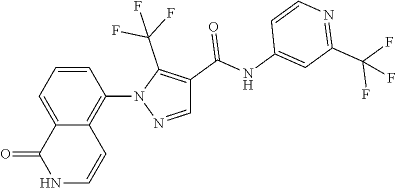 [SMILES: O=C(Nc1ccnc(C(F)(F)F)c1)c1cnn(-c2cccc3c(=O)[nH]ccc23)c1C(F)(F)F]

or a pharmaceutically acceptable salt form thereof to said patient.

Embodiment 65. The method of embodiment 64, wherein the therapeutically effective dose is:
from about 50 to about 500 mg;
from about 100 to about 500 mg; or
from about 100 to about 400 mg.

Embodiment 66. The method of embodiment 64, wherein the therapeutically effective dose is:
from about 150 to about 350 mg;
from about 200 to about 350 mg;
from about 275 to about 375 mg; or about 300 mg.

Embodiment 67. The method of any one of embodiments 64-66, wherein the therapeutically effective dose is divided in half, said half dose being administered twice (two times) a day.

Embodiment 68. The method of any one of embodiments 64-66, wherein the therapeutically effective dose is administered one time a day.

Embodiment 69. The method of any one of embodiments 64-68, wherein the therapeutically effective dose is administered daily on a continuous 28-day cycle.

Embodiment 70. The method of any one of embodiments 64-68, wherein the therapeutically effective dose is administered daily on a continuous 7-day to 21-day cycle.

Embodiment 71. The method of embodiments 69 or 70, wherein the cycle is repeated.

Embodiment 72. The method of any one of embodiments 64-71, wherein the method further comprises determining the proportion of CD8⁺ T_{eff} and CD4⁺CD25^{hi}FOXP3^{hi} T_{reg} cells.

Embodiment 73. 1-(1-oxo-1,2-dihydroisoquinolin-5-yl)-5-(trifluoromethyl)-N-[2-(trifluoromethyl)pyridin-4-yl]-1H-pyrazole-4-carboxamide (Compound A):

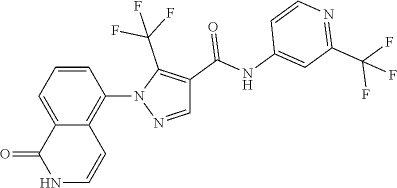 [SMILES: O=C(Nc1ccnc(C(F)(F)F)c1)c1cnn(-c2cccc3c(=O)[nH]ccc23)c1C(F)(F)F]

or a pharmaceutically acceptable salt form thereof for use in treating a disorder or condition that is affected by the inhibition of MALT1 in a subject in need of treatment, comprising administering a therapeutically effective dose ranging from about 50 mg to about 1000 mg of Compound A or a pharmaceutically acceptable salt form thereof to said subject.

Embodiment 74. Compound A or a pharmaceutically acceptable salt form thereof for the use of embodiment 73, wherein the subject is a human.

Embodiment 75. Compound A or a pharmaceutically acceptable salt form thereof for the use of embodiment 73 or 74, wherein the therapeutically effective dose is from about 50 to about 500 mg.

Embodiment 76. Compound A or a pharmaceutically acceptable salt form thereof for the use of embodiment 73 or 74, wherein the therapeutically effective dose is from about 100 to about 400 mg.

Embodiment 77. Compound A or a pharmaceutically acceptable salt form thereof for the use of embodiment 73 or 74, wherein the therapeutically effective dose is from about 150 to about 300 mg.

Embodiment 78. Compound A or a pharmaceutically acceptable salt form thereof for the use of embodiment 73 or 74, wherein the therapeutically effective dose is about 300 mg.

Embodiment 79. Compound A or a pharmaceutically acceptable salt form thereof for the use of embodiment 73 or 74, wherein the therapeutically effective dose is from about 100 to about 150 mg.

Embodiment 80. Compound A or a pharmaceutically acceptable salt form thereof for the use of embodiment 73 or 74, wherein the therapeutically effective dose is from about 150 to about 200 mg.

Embodiment 81. Compound A or a pharmaceutically acceptable salt form thereof for the use of embodiment 73 or 74, wherein the therapeutically effective dose is from about 200 to about 250 mg.

Embodiment 82. Compound A or a pharmaceutically acceptable salt form thereof for the use of embodiment 73 or 74, wherein the therapeutically effective dose is from about 250 to about 300 mg.

Embodiment 83. Compound A or a pharmaceutically acceptable salt form thereof for the use of embodiment 73 or 74, wherein the therapeutically effective dose is from about 300 to 350 mg.

Embodiment 84. Compound A or a pharmaceutically acceptable salt form thereof for the use of embodiment 73 or 74, wherein the therapeutically effective dose is from about 350 to 400 mg.

Embodiment 85. Compound A or a pharmaceutically acceptable salt form thereof for the use of any one of embodiments 73-84, wherein the therapeutically effective dose is divided in half, said half dose being administered twice (two times) a day.

Embodiment 86. Compound A or a pharmaceutically acceptable salt form thereof for the use of any one of embodiments 73-84, wherein the therapeutically effective dose is administered one time a day.

Embodiment 87. Compound A or a pharmaceutically acceptable salt form thereof for the use of any one of embodiments 73-86, wherein the therapeutically effective dose is administered daily on a continuous 28-day cycle.

Embodiment 88. Compound A or a pharmaceutically acceptable salt form thereof for the use of any one of embodiments 73-86, wherein the therapeutically effective dose is administered daily on a continuous 21-day cycle.

Embodiment 89. 1-(1-oxo-1,2-dihydroisoquinolin-5-yl)-5-(trifluoromethyl)-N-[2-(trifluoromethyl)pyridin-4-yl]-1H-pyrazole-4-carboxamide (Compound A):

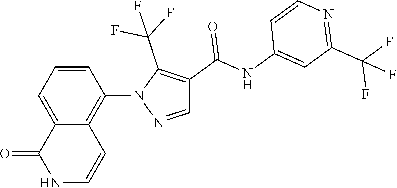 [SMILES: O=C(Nc1ccnc(C(F)(F)F)c1)c1cnn(-c2cccc3c(=O)[nH]ccc23)c1C(F)(F)F]

or a pharmaceutically acceptable salt form thereof for use in treating a disorder or condition that is affected by the inhibition of MALT1 in a subject in need of treatment, comprising administering a therapeutic effective dose of Compound A or a pharmaceutically acceptable salt form thereof to said subject in an amount sufficient to maintain a plasma level of Compound A from about 2 μg/ml to about 120 μg/ml, about 2 μg/ml to about 100 μg/ml, about 2 μg/ml to about 80 μg/ml, about 2 μg/ml to about 60 μg/ml, or about 2 μg/ml to about 20 μg/ml.

Embodiment 90. 1-(1-oxo-1,2-dihydroisoquinolin-5-yl)-5-(trifluoromethyl)-N-[2-(trifluoromethyl)pyridin-4-yl]-1H-pyrazole-4-carboxamide (Compound A):

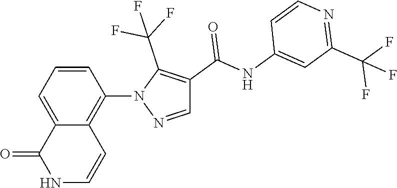 [SMILES: O=C(Nc1ccnc(C(F)(F)F)c1)c1cnn(-c2cccc3c(=O)[nH]ccc23)c1C(F)(F)F]

or a pharmaceutically acceptable salt form thereof for use in treating a disorder or condition that is affected by the inhibition of MALT1 in a subject in need of treatment, comprising administering a therapeutic effective dose of Compound A or a pharmaceutically acceptable salt form thereof to said subject in an amount sufficient to achieve an AUC from about 50 μg·h/ml to about 2500 μg·h/ml, about 50 μg·h/ml to about 2000 μg·h/ml, about 50 μg·h/ml to about 1500 μg·h/ml, about 50 μg·h/ml to about 1000 μg·h/ml, or about 50 μg·h/ml to about 600 μg·h/ml.

Embodiment 91. 1-(1-oxo-1,2-dihydroisoquinolin-5-yl)-5-(trifluoromethyl)-N-[2-(trifluoromethyl)pyridin-4-yl]-1H-pyrazole-4-carboxamide (Compound A):

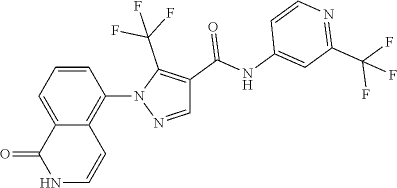 [SMILES: O=C(Nc1ccnc(C(F)(F)F)c1)c1cnn(-c2cccc3c(=O)[nH]ccc23)c1C(F)(F)F]

or a pharmaceutically acceptable salt form thereof for use in treating a cancer or an immunological disease in a subject in need of treatment, comprising administering a therapeutic effective dose of Compound A or a pharmaceutically acceptable salt form thereof to said subject in an amount sufficient to maintain a plasma level of Compound A from about 2 μg/ml to about 120 μg/ml, about 2 μg/ml to about 100 μg/ml, about 2 μg/ml to about 80 μg/ml, about 2 μg/ml to about 60 μg/ml, or about 2 μg/ml to about 20 μg/ml.

Embodiment 92. 1-(1-oxo-1,2-dihydroisoquinolin-5-yl)-5-(trifluoromethyl)-N-[2-(trifluoromethyl)pyridin-4-yl]-1H-pyrazole-4-carboxamide (Compound A):

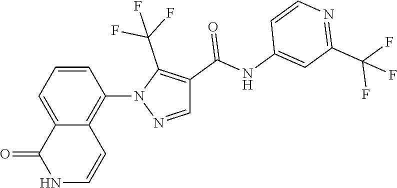 [SMILES: O=C(Nc1ccnc(C(F)(F)F)c1)c1cnn(-c2cccc3c(=O)[nH]ccc23)c1C(F)(F)F]

or a pharmaceutically acceptable salt form thereof for use in treating a cancer or an immunological disease in a subject in need of treatment, comprising administering Compound A or a pharmaceutically acceptable salt to said subject in an amount sufficient to achieve an AUC from about 50 μg·h/ml to about 2500 μg·h/ml, about 50 μg·h/ml to about 2000 μg·h/ml, about 50 μg·h/ml to about 1500 μg·h/ml, about 50 μg·h/ml to about 1000 μg·h/ml, or about 50 μg·h/ml to about 600 μg·h/ml.

Embodiment 93. Compound A or a pharmaceutically acceptable salt form thereof for use of any one of embodiments 73-92, wherein the disorder or condition is a cancer selected from lymphomas, leukemias, carcinomas, and sarcomas, e.g. non-Hodgkin's lymphoma (NHL (including B-cell NHL)), diffuse large B-cell lymphoma (DLBCL), mantle cell lymphoma (MCL), follicular lymphoma (FL), mucosa-associated lymphoid tissue (MALT) lymphoma, marginal zone lymphoma, T-cell lymphoma, Hodgkin's lymphoma, Burkitt's lymphoma, multiple myeloma, chronic lymphocytic leukemia (CLL), small lymphocytic lymphoma (SLL), Waldenström macroglobulinemia, lymphoblastic T cell leukemia, chronic myelogenous leukemia (CML), hairy-cell leukemia, acute lymphoblastic T cell leukemia, plasmacytoma, immunoblastic large cell leukemia, megakaryoblastic leukemia, acute megakaryocyte leukemia, promyelocytic leukemia, erythroleukemia, brain (gliomas), glioblastomas, breast cancer, colorectal/colon cancer, prostate cancer, lung cancer including non-small-cell, gastric cancer, endometrial cancer, melanoma, pancreatic cancer, liver cancer, kidney cancer, squamous cell carcinoma, ovarian cancer, sarcoma, osteosarcoma, thyroid cancer, bladder cancer, head & neck cancer, testicular cancer, Ewing's sarcoma, rhabdomyosarcoma, medulloblastoma, neuroblastoma, cervical cancer, renal cancer, urothelial cancer, vulval cancer, esophageal cancer, salivary gland cancer, nasopharyngeal cancer, buccal cancer, cancer of the mouth, primary and secondary central nervous system lymphoma, transformed follicular lymphoma, diseases/cancer caused by API2-MALT1 fusion, and GIST (gastrointestinal stromal tumor).

Embodiment 94. Compound A or a pharmaceutically acceptable salt form thereof for use of any one of embodiments 73-92, wherein the disorder or condition is an immunological disease selected from autoimmune and inflammatory disorders, e.g. arthritis, rheumatoid arthritis (RA), psoriatic arthritis (PsA), inflammatory bowel disease, gastritis, ankylosing spondylitis, ulcerative colitis, pancreatitis, Crohn's disease, celiac disease, multiple sclerosis, systemic lupus erythematosus, lupus nephritis, rheumatic fever, gout, organ or transplant rejection, chronic allograft rejection, acute or chronic graft-versus-host disease, dermatitis including atopic, dermatomyositis, psoriasis, Behcet's diseases, uveitis, myasthenia gravis, Grave's disease, Hashimoto thyroiditis, Sjoergen's syndrome, blistering disorders, antibody-mediated vasculitis syndromes, immune-complex vasculitides, allergic disorders, asthma, bronchitis, chronic obstructive pulmonary disease (COPD), cystic fibrosis, pneumonia, pulmonary diseases including oedema, embolism, fibrosis, sarcoidosis, hypertension and emphysema, silicosis, respiratory failure, acute respiratory distress syndrome, BENTA disease, berylliosis, and polymyositis.

Embodiment 95. Compound A or a pharmaceutically acceptable salt form thereof for use of any one of embodiments 73-92, wherein the disorder or condition is selected from non-Hodgkin's lymphoma (NHL), diffuse large B-cell lymphoma (DLBCL), marginal zone lymphoma, mantle cell lymphoma (MCL), follicular lymphoma (FL), transformed follicular lymphoma, chronic lymphocytic leukemia, and Waldenström macroglobulinemia.

Embodiment 96. Compound A for use in treating non-Hodgkin's lymphoma (NHL) in a subject comprising administering to the subject a therapeutically effective dose of about 100 mg to about 300 mg of Compound A.

Embodiment 97. Compound A for use in treating diffuse large B-cell lymphoma (DLBCL) in a subject comprising administering to the subject a therapeutically effective dose of about 100 mg to about 300 mg of Compound A.

Embodiment 98. Compound A for use in treating marginal zone lymphoma (MZL) in a subject comprising administering to the subject a therapeutically effective dose of about 100 mg to about 300 mg of Compound A.

Embodiment 99. Compound A for use in treating mantle cell lymphoma (MCL) in a subject comprising administering to the subject a therapeutically effective dose of about 100 mg to about 300 mg of Compound A.

Embodiment 100. Compound A for use in treating follicular lymphoma (FL) in a subject comprising administering to the subject a therapeutically effective dose of about 100 mg to about 300 mg of Compound A.

Embodiment 101. Compound A for use in treating transformed follicular lymphoma (tFL) in a subject comprising administering to the subject a therapeutically effective dose of about 100 mg to about 300 mg of Compound A.

Embodiment 102. Compound A for use in treating chronic lymphocytic leukemia (CLL) in a subject comprising administering to the subject a therapeutically effective dose of about 100 mg to about 300 mg of Compound A.

Embodiment 103. Compound A for use in treating Waldenström macroglobulinemia in a subject comprising administering to the subject a therapeutically effective dose of about 100 mg to about 300 mg of Compound A.

Embodiment 104. Compound A or a pharmaceutically acceptable salt form thereof for use of embodiment 97, wherein the DLBCL is the activated B cell like (ABC) subtype of diffuse large B-cell lymphoma (DLBCL).

Embodiment 105. Compound A or a pharmaceutically acceptable salt form thereof for use of embodiment 97, wherein the DLBCL is germinal center B cell like (GCB) subtype of diffuse large B-cell lymphoma (DLBCL).

Embodiment 106. Compound A or a pharmaceutically acceptable salt form thereof for use of embodiment 97, wherein the DLBCL is non-germinal center B cell like (non-GCB) subtype of diffuse large B-cell lymphoma (DLBCL).

Embodiment 107. Compound A or a pharmaceutically acceptable salt form thereof for use of any one of embodiments 96-106, wherein the method comprises administering about 100 mg to about 300 mg of Compound A once daily for 7 to 21 continuous day cycle, and optionally 3-10 cycles.

Embodiment 108. Compound A or a pharmaceutically acceptable salt form thereof for use of any one of embodiments 96-106, wherein the method comprises administering about 100 mg to about 300 mg of Compound A twice daily for 7 days followed by administration of about 100 mg to about 300 mg of Compound A once a day for 14 days, and optionally 3-10 cycles.

Embodiment 109. Compound A or a pharmaceutically acceptable salt form thereof for use of any one of embodiments 96-106, wherein the method comprises administering a therapeutically effective dose 100 mg to about 300 mg of Compound A twice daily for 7 days followed by administration of about 100 mg to about 300 mg of Compound A once a day until remission.

Embodiment 110. Compound A or a pharmaceutically acceptable salt form thereof for use of any one of embodiments 96-106, wherein said subject has received prior treatment with a Bruton tyrosine kinase inhibitor (BTKi).

Embodiment 111. Compound A or a pharmaceutically acceptable salt form thereof for use of any one of embodiments 96-106, wherein said subject is relapsed or refractory to prior treatment with a Bruton tyrosine kinase inhibitor (BTKi).

Embodiment 112. Compound A or a pharmaceutically acceptable salt form thereof for use of any one of embodiments 73-111, wherein Compound A is used as a hydrate or a monohydrate form thereof.

Embodiment 113. Compound A or a pharmaceutically acceptable salt form thereof for use of any one of embodiments 73-112, wherein said subject is administered a pharmaceutical composition of Compound A or a pharmaceutically acceptable salt form thereof further comprising a pharmaceutically acceptable carrier, a pharmaceutically acceptable excipient, and/or a pharmaceutically acceptable diluent.

Embodiment 114. Compound A or a pharmaceutically acceptable salt form thereof for use of any one of embodiments 73-88, wherein the use comprises: an amount sufficient to maintain a plasma level of Compound A from about 2,300 ng/mL to about 9,300 ng/mL; an amount sufficient to maintain a plasma level of Compound A from about 2,320 ng/ml to about 9,280 ng/ml; an amount sufficient to maintain a plasma level of Compound A from about 3,000 ng/mL to about 9,000 ng/ml; an amount sufficient to maintain a plasma level of Compound A from about 3,500 ng/ml to about 8,500 ng/ml; an amount sufficient to maintain a plasma level of Compound A from about 4,000 ng/mL to about 8,000 ng/ml; an amount sufficient to maintain a plasma level of Compound A from about 4,000 ng/mL to about 6,000 ng/ml; an amount sufficient to maintain a plasma level of Compound A of at least 4,600 ng/ml; an amount sufficient to maintain a plasma level of Compound A from about 4,500 ng/mL to about 4,750 ng/mL; an amount sufficient to maintain a plasma level of Compound A from about 4,640 ng/ml; an amount sufficient to maintain a plasma level of Compound A from about 4,550 to about 4,700 ng/ml; or an amount sufficient to maintain a plasma level of Compound A from about 4,550 to about 4,680 ng/ml.

Embodiment 115. Compound A or a pharmaceutically acceptable salt form thereof for use of any one of embodiments 73-88 or 114, wherein Compound A is used as a hydrate or a monohydrate form thereof.

Embodiment 116. Compound A or a pharmaceutically acceptable salt form thereof for use of any one of embodiments 73-88 or 114-115, wherein the use comprises a pharmaceutical composition of Compound A or a solvate or pharmaceutically acceptable salt form thereof further comprising a pharmaceutically acceptable carrier, a pharmaceutically acceptable excipient, and/or a pharmaceutically acceptable diluent.

Embodiment 117. Compound A or a pharmaceutically acceptable salt form thereof for use of any one of embodiments 73-88 or 114-116, wherein said disorder or condition is a cancer selected from lymphomas, leukemias, carcinomas, and sarcomas, e.g. non-Hodgkin's lymphoma (NHL (including B-cell NHL)), diffuse large B-cell lymphoma (DLBCL), mantle cell lymphoma (MCL), follicular lymphoma (FL), mucosa-associated lymphoid tissue (MALT) lymphoma, marginal zone lymphoma, T-cell lymphoma, Hodgkin's lymphoma, Burkitt's lymphoma, multiple myeloma, chronic lymphocytic leukemia (CLL), small lymphocytic lymphoma (SLL), Waldenström macroglobulinemia, lymphoblastic T cell leukemia, chronic myelogenous leukemia (CML), hairy-cell leukemia, acute lymphoblastic T cell leukemia, plasmacytoma, immunoblastic large cell leukemia, megakaryoblastic leukemia, acute megakaryocyte leukemia, promyelocytic leukemia, erythroleukemia, brain (gliomas), glioblastomas, breast cancer, colorectal/colon cancer, prostate cancer, lung cancer including non-small-cell, gastric cancer, endometrial cancer, melanoma, pancreatic cancer, liver cancer, kidney cancer, squamous cell carcinoma, ovarian cancer, sarcoma, osteosarcoma, thyroid cancer, bladder cancer, head & neck cancer, testicular cancer, Ewing's sarcoma, rhabdomyosarcoma, medulloblastoma, neuroblastoma, cervical cancer, renal cancer, urothelial cancer, vulval cancer, esophageal cancer, salivary gland cancer, nasopharyngeal cancer, buccal cancer, cancer of the mouth, primary and secondary central nervous system lymphoma, transformed follicular lymphoma, diseases/cancer caused by API2-MALT1 fusion, and GIST (gastrointestinal stromal tumor).

Embodiment 118. Compound A or a pharmaceutically acceptable salt form thereof for use of any one of embodiments 73-88 or 114-116, wherein said disorder or condition is an immunological disease selected from autoimmune and inflammatory disorders, e.g. arthritis, rheumatoid arthritis (RA), psoriatic arthritis (PsA), inflammatory bowel disease, gastritis, ankylosing spondylitis, ulcerative colitis, pancreatitis, Crohn's disease, celiac disease, multiple sclerosis, systemic lupus erythematosus, lupus nephritis, rheumatic fever, gout, organ or transplant rejection, chronic allograft rejection, acute or chronic graft-versus-host disease, dermatitis including atopic, dermatomyositis, psoriasis, Behcet's diseases, uveitis, myasthenia gravis, Grave's disease, Hashimoto thyroiditis, Sjoergen's syndrome, blistering disorders, antibody-mediated vasculitis syndromes, immune-complex vasculitides, allergic disorders, asthma, bronchitis, chronic obstructive pulmonary disease (COPD), cystic fibrosis, pneumonia, pulmonary diseases including oedema, embolism, fibrosis, sarcoidosis, hypertension and emphysema, silicosis, respiratory failure, acute respiratory distress syndrome, BENTA disease, berylliosis, and polymyositis.

Embodiment 119. Compound A or a pharmaceutically acceptable salt form thereof for use of any one of embodiments 73-88 or 114-118, wherein said disorder or condition is relapsed or refractory to prior treatment

Embodiment 120. Compound A or a pharmaceutically acceptable salt form thereof for use of any one of embodiments 73-88 or 114-118, wherein said disorder or condition is relapsed or refractory to prior treatment with a Bruton tyrosine kinase inhibitor (BTKi).

Embodiment 121. Compound A or a pharmaceutically acceptable salt form thereof for use in a method of reducing the T_{reg}/T_{eff} ratio in a patient suffering from a disorder or condition that is affected by the inhibition of MALT1 comprising administering a therapeutically effective dose of Compound A or a pharmaceutically acceptable salt form thereof to said patient.

Embodiment 122. Compound A or a pharmaceutically acceptable salt form thereof for use of embodiment 121, wherein the therapeutically effective dose is:
from about 50 to about 500 mg;
from about 100 to about 500 mg; or
from about 100 to about 400 mg.

Embodiment 123. Compound A or a pharmaceutically acceptable salt form thereof for use of embodiment 121, wherein the therapeutically effective dose is:
from about 150 to about 350 mg;
from about 200 to about 350 mg;
from about 275 to about 375 mg; or about 300 mg.

Embodiment 124. Compound A or a pharmaceutically acceptable salt form thereof for use of any one of embodiments 121-123, wherein the therapeutically effective dose is divided in half, said half dose being administered twice (two times) a day.

Embodiment 125. Compound A or a pharmaceutically acceptable salt form thereof for use of any one of embodiments 121-123, wherein the therapeutically effective dose is administered one time a day.

Embodiment 126. Compound A or a pharmaceutically acceptable salt form thereof for use of any one of embodiments 121-125, wherein the therapeutically effective dose is administered daily on a continuous 28-day cycle.

Embodiment 127. Compound A or a pharmaceutically acceptable salt form thereof for use of any one of embodiments 121-125, wherein the therapeutically effective dose is administered daily on a continuous 7-day to 21-day cycle.

Embodiment 128. Compound A or a pharmaceutically acceptable salt form thereof for use of embodiments 126 or 127, wherein the cycle is repeated.

Embodiment 129. Compound A or a pharmaceutically acceptable salt form thereof for use of any one of embodiments 121-127, wherein the method further comprises determining the proportion of CD8⁺ T_{eff} and CD4⁺CD25^{hi}FOXP3^{hi} T_{reg} cells.

Embodiment 130. The use of 1-(1-oxo-1,2-dihydroisoquinolin-5-yl)-5-(trifluoromethyl)-N-[2-(trifluoromethyl)pyridin-4-yl]-1H-pyrazole-4-carboxamide (Compound A):

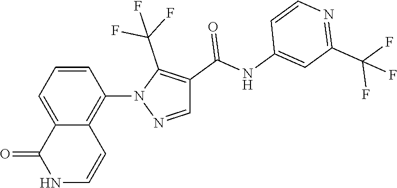 [SMILES: O=C(Nc1ccnc(C(F)(F)F)c1)c1cnn(-c2cccc3c(=O)[nH]ccc23)c1C(F)(F)F]

or a pharmaceutically acceptable salt form thereof for the manufacture of a medicament for the treatment of a disorder or condition that is affected by the inhibition of MALT1 in a subject in need of treatment, comprising administering a therapeutically effective dose ranging from about 50 mg to about 1000 mg of Compound A or a pharmaceutically acceptable salt form thereof to said subject.

Embodiment 131. The use of embodiment 130, wherein the subject is a human.

Embodiment 132. The use of embodiment 130 or 131, wherein the therapeutically effective dose is from about 50 to about 500 mg.

Embodiment 133. The use of embodiment 130 or 131, wherein the therapeutically effective dose is from about 100 to about 400 mg.

Embodiment 134. The use of embodiment 130 or 131, wherein the therapeutically effective dose is from about 150 to about 300 mg.

Embodiment 135. The use of embodiment 130 or 131, wherein the therapeutically effective dose is about 300 mg.

Embodiment 136. The use of embodiment 130 or 131, wherein the therapeutically effective dose is from about 100 to about 150 mg.

Embodiment 137. The use of embodiment 130 or 131, wherein the therapeutically effective dose is from about 150 to about 200 mg.

Embodiment 138. The use of embodiment 130 or 131, wherein the therapeutically effective dose is from about 200 to about 250 mg.

Embodiment 139. The use of embodiment 130 or 131, wherein the therapeutically effective dose is from about 250 to about 300 mg.

Embodiment 140. The use of embodiment 130 or 131, wherein the therapeutically effective dose is from about 300 to 350 mg.

Embodiment 141. The use of embodiment 130 or 131, wherein the therapeutically effective dose is from about 350 to 400 mg.

Embodiment 142. The use of any one of embodiments 130-141, wherein the therapeutically effective dose is divided in half, said half dose being administered twice (two times) a day.

Embodiment 143. The use of any one of embodiments 130-141, wherein the therapeutically effective dose is administered one time a day.

Embodiment 144. The use of any one of embodiments 130-143, wherein the therapeutically effective dose is administered daily on a continuous 28-day cycle.

Embodiment 145. The use of any one of embodiments 130-143, wherein the therapeutically effective dose is administered daily on a continuous 21-day cycle.

Embodiment 146. The use of 1-(1-oxo-1,2-dihydroisoquinolin-5-yl)-5-(trifluoromethyl)-N-[2-(trifluoromethyl)pyridin-4-yl]-1H-pyrazole-4-carboxamide (Compound A):

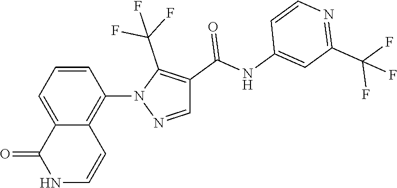 [SMILES: O=C(Nc1ccnc(C(F)(F)F)c1)c1cnn(-c2cccc3c(=O)[nH]ccc23)c1C(F)(F)F]

or a pharmaceutically acceptable salt form thereof for the manufacture of a medicament for the treatment of a disorder or condition that is affected by the inhibition of MALT1 in a subject in need of treatment, comprising administering a therapeutic effective dose of Compound A or a pharmaceutically acceptable salt form thereof to said subject in an amount sufficient to maintain a plasma level of Compound A from about 2 μg/ml to about 120 μg/ml, about 2 μg/ml to about 100 μg/ml, about 2 μg/ml to about 80 μg/ml, about 2 μg/ml to about 60 μg/ml, or about 2 μg/ml to about 20 μg/ml.

Embodiment 147. The use of 1-(1-oxo-1,2-dihydroisoquinolin-5-yl)-5-(trifluoromethyl)-N-[2-(trifluoromethyl)pyridin-4-yl]-1H-pyrazole-4-carboxamide (Compound A):

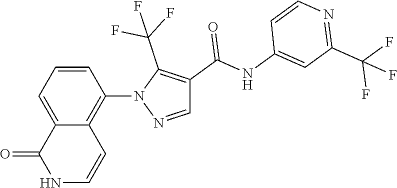 [SMILES: O=C(Nc1ccnc(C(F)(F)F)c1)c1cnn(-c2cccc3c(=O)[nH]ccc23)c1C(F)(F)F]

or a pharmaceutically acceptable salt form thereof for the manufacture of a medicament for the treatment of a disorder or condition that is affected by the inhibition of MALT1 in a subject in need of treatment, comprising administering a therapeutic effective dose of Compound A or a pharmaceutically acceptable salt form thereof to said subject in an amount sufficient to achieve an AUC from about 50 μg·h/ml to about 2500 μg·h/ml, about 50 μg·h/ml to about 2000 μg·h/ml, about 50 μg·h/ml to about 1500 μg·h/ml, about 50 μg·h/ml to about 1000 μg·h/ml, or about 50 μg·h/ml to about 600 μg·h/ml.

Embodiment 148. The use of 1-(1-oxo-1,2-dihydroisoquinolin-5-yl)-5-(trifluoromethyl)-N-[2-(trifluoromethyl)pyridin-4-yl]-1H-pyrazole-4-carboxamide (Compound A):

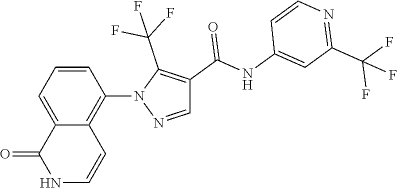 [SMILES: O=C(Nc1ccnc(C(F)(F)F)c1)c1cnn(-c2cccc3c(=O)[nH]ccc23)c1C(F)(F)F]

or a pharmaceutically acceptable salt form thereof for the manufacture of a medicament for the treatment of a cancer or an immunological disease in a subject in need of treatment, comprising administering a therapeutic effective dose of Compound A or a pharmaceutically acceptable salt form thereof to said subject in an amount sufficient to maintain a plasma level of Compound A from about 2 μg/ml to about 120 μg/ml, about 2 μg/ml to about 100 μg/ml, about 2 μg/ml to about 80 μg/ml, about 2 μg/ml to about 60 μg/ml, or about 2 μg/ml to about 20 μg/ml.

Embodiment 149. The use of 1-(1-oxo-1,2-dihydroisoquinolin-5-yl)-5-(trifluoromethyl)-N-[2-(trifluoromethyl)pyridin-4-yl]-1H-pyrazole-4-carboxamide (Compound A):

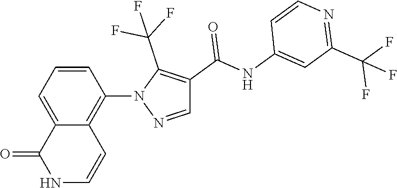 [SMILES: O=C(Nc1ccnc(C(F)(F)F)c1)c1cnn(-c2cccc3c(=O)[nH]ccc23)c1C(F)(F)F]

or a pharmaceutically acceptable salt form thereof for the manufacture of a medicament for the treatment of a cancer or an immunological disease in a subject in need of treatment, comprising administering Compound A or a pharmaceutically acceptable salt form thereof to said subject in an amount sufficient to achieve an AUC from about 50 μg·h/ml to about 2500 μg·h/ml, about 50 μg·h/ml to about 2000 μg·h/ml, about 50 μg·h/ml to about 1500 μg·h/ml, about 50 μg·h/ml to about 1000 μg·h/ml, or about 50 μg·h/ml to about 600 μg·h/ml.

Embodiment 150. The use of any one of embodiments 130-149, wherein the disorder or condition is a cancer selected from lymphomas, leukemias, carcinomas, and sarcomas, e.g. non-Hodgkin's lymphoma (NHL (including B-cell NHL)), diffuse large B-cell lymphoma (DLBCL), mantle cell lymphoma (MCL), follicular lymphoma (FL), mucosa-associated lymphoid tissue (MALT) lymphoma, marginal zone lymphoma, T-cell lymphoma, Hodgkin's lymphoma, Burkitt's lymphoma, multiple myeloma, chronic lymphocytic leukemia (CLL), small lymphocytic lymphoma (SLL), Waldenström macroglobulinemia, lymphoblastic T cell leukemia, chronic myelogenous leukemia (CML), hairy-cell leukemia, acute lymphoblastic T cell leukemia, plasmacytoma, immunoblastic large cell leukemia, megakaryoblastic leukemia, acute megakaryocyte leukemia, promyelocytic leukemia, erythroleukemia, brain (gliomas), glioblastomas, breast cancer, colorectal/colon cancer, prostate cancer, lung cancer including non-small-cell, gastric cancer, endometrial cancer, melanoma, pancreatic cancer, liver cancer, kidney cancer, squamous cell carcinoma, ovarian cancer, sarcoma, osteosarcoma, thyroid cancer, bladder cancer, head & neck cancer, testicular cancer, Ewing's sarcoma, rhabdomyosarcoma, medulloblastoma, neuroblastoma, cervical cancer, renal cancer, urothelial cancer, vulval cancer, esophageal cancer, salivary gland cancer, nasopharyngeal cancer, buccal cancer, cancer of the mouth, primary and secondary central nervous system lymphoma, transformed follicular lymphoma, diseases/cancer caused by API2-MALT1 fusion, and GIST (gastrointestinal stromal tumor).

Embodiment 151. The use of any one of embodiments 130-149, wherein the the disorder or condition is an immunological disease selected from autoimmune and inflammatory disorders, e.g. arthritis, rheumatoid arthritis (RA), psoriatic arthritis (PsA), inflammatory bowel disease, gastritis, ankylosing spondylitis, ulcerative colitis, pancreatitis, Crohn's disease, celiac disease, multiple sclerosis, systemic lupus erythematosus, lupus nephritis, rheumatic fever, gout, organ or transplant rejection, chronic allograft rejection, acute or chronic graft-versus-host disease, dermatitis including atopic, dermatomyositis, psoriasis, Behcet's diseases, uveitis, myasthenia gravis, Grave's disease, Hashimoto thyroiditis, Sjoergen's syndrome, blistering disorders, antibody-mediated vasculitis syndromes, immune-complex vasculitides, allergic disorders, asthma, bronchitis, chronic obstructive pulmonary disease (COPD), cystic fibrosis, pneumonia, pulmonary diseases including oedema, embolism, fibrosis, sarcoidosis, hypertension and emphysema, silicosis, respiratory failure, acute respiratory distress syndrome, BENTA disease, berylliosis, and polymyositis.

Embodiment 152. The use of any one of embodiments 130-149, wherein the disorder or condition is selected from non-Hodgkin's lymphoma (NHL), diffuse large B-cell lymphoma (DLBCL), marginal zone lymphoma, mantle cell lymphoma (MCL), follicular lymphoma (FL), transformed follicular lymphoma, chronic lymphocytic leukemia, and Waldenström macroglobulinemia.

Embodiment 153. The use of compound A for the manufacture of a medicament for the treatment of non-Hodgkin's lymphoma (NHL) in a subject comprising administering to the subject a therapeutically effective dose of about 100 mg to about 300 mg of Compound A.

Embodiment 154. The use of compound A for the manufacture of a medicament for the treatment of diffuse large B-cell lymphoma (DLBCL) in a subject comprising administering to the subject a therapeutically effective dose of about 100 mg to about 300 mg of Compound A.

Embodiment 155. The use of compound A for the manufacture of a medicament for the treatment of marginal zone lymphoma (MZL) in a subject comprising administering to the subject a therapeutically effective dose of about 100 mg to about 300 mg of Compound A.

Embodiment 156. The use of compound A for the manufacture of a medicament for the treatment of mantle cell lymphoma (MCL) in a subject comprising administering to the subject a therapeutically effective dose of about 100 mg to about 300 mg of Compound A.

Embodiment 157. The use of compound A for the manufacture of a medicament for the treatment of follicular lymphoma (FL) in a subject comprising administering to the subject a therapeutically effective dose of about 100 mg to about 300 mg of Compound A.

Embodiment 158. The use of compound A for the manufacture of a medicament for the treatment of transformed follicular lymphoma (tFL) in a subject comprising administering to the subject a therapeutically effective dose of about 100 mg to about 300 mg of Compound A.

Embodiment 159. The use of compound A for the manufacture of a medicament for the treatment of chronic lymphocytic leukemia (CLL) in a subject comprising administering to the subject a therapeutically effective dose of about 100 mg to about 300 mg of Compound A.

Embodiment 160. The use of compound A for the manufacture of a medicament for the treatment of Waldenström macroglobulinemia in a subject comprising administering to the subject a therapeutically effective dose of about 100 mg to about 300 mg of Compound A.

Embodiment 161. The use of embodiment 154, wherein the DLBCL is the activated B cell like (ABC) subtype of diffuse large B-cell lymphoma (DLBCL).

Embodiment 162. The use of embodiment 154, wherein the DLBCL is germinal center B cell like (GCB) subtype of diffuse large B-cell lymphoma (DLBCL).

Embodiment 163. The use of embodiment 154, wherein the DLBCL is non-germinal center B cell like (non-GCB) subtype of diffuse large B-cell lymphoma (DLBCL).

Embodiment 164. The use of any one of embodiments 153-163, wherein the use comprises administering about 100 mg to about 300 mg of Compound A once daily for 7 to 21 continuous day cycle, and optionally 3-10 cycles.

Embodiment 165. The use of any one of embodiments 153-163, wherein the use comprises administering about 100 mg to about 300 mg of Compound A twice daily for 7 days followed by administration of about 100 mg to about 300 mg of Compound A once a day for 14 days, and optionally 3-10 cycles.

Embodiment 166. The use of any one of embodiments 153-163, wherein the use comprises administering a therapeutically effective dose 100 mg to about 300 mg of Compound A twice daily for 7 days followed by administration of about 100 mg to about 300 mg of Compound A once a day until remission.

Embodiment 167. The use of any one of embodiments 153-163, wherein said subject has received prior treatment with a Bruton tyrosine kinase inhibitor (BTKi).

Embodiment 168. The use of any one of embodiments 153-163, wherein said subject is relapsed or refractory to prior treatment with a Bruton tyrosine kinase inhibitor (BTKi).

Embodiment 169. The use of any one of embodiments 130-168, wherein Compound A is used as a hydrate or a monohydrate form thereof.

Embodiment 170. The use of any one of embodiments 130-169, wherein said subject is administered a pharmaceutical composition of Compound A or a pharmaceutically acceptable salt form thereof further comprising a pharmaceutically acceptable carrier, a pharmaceutically acceptable excipient, and/or a pharmaceutically acceptable diluent.

Embodiment 171. The use of 1-(1-oxo-1,2-dihydroisoquinolin-5-yl)-5-trifluoromethyl)-N-[2-(trifluoromethyl)pyridin-4-yl]-1H-pyrazole-4-carboxamide (Compound A):

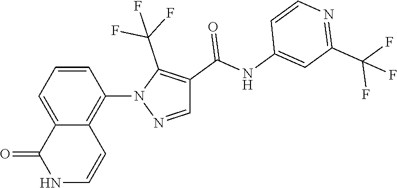 [SMILES: O=C(Nc1ccnc(C(F)(F)F)c1)c1cnn(-c2cccc3c(=O)[nH]ccc23)c1C(F)(F)F]

or a pharmaceutically acceptable salt form thereof for the manufacture of a medicament for the treatment of a disorder or condition that is affected by the inhibition of MALT1, wherein the use comprises a therapeutically effective dose of from about 50 mg to about 1000 mg of Compound A.

Embodiment 172. The use of embodiment 171 wherein the use comprises: a therapeutically effective dose of from about 50 to about 1000 mg; a therapeutically effective dose of from about 50 to about 500 mg; a therapeutically effective dose of from about 100 to about 400 mg; a therapeutically effective dose of from about 150 to about 300 mg; a therapeutically effective dose of about 200 mg; a therapeutically effective dose of from about 100 to about 150 mg; a therapeutically effective dose of from about 150 to about 200 mg; a therapeutically effective dose of from about 200 to about 250 mg; a therapeutically effective dose of from about 250 to about 300 mg; a therapeutically effective dose of from about 300 to 350 mg; or a therapeutically effective dose of from about 350 to 400 mg.

Embodiment 173. The use of embodiments 171 or 172, wherein the therapeutically effective dose is divided in half, said half dose being administered twice (two times) a day.

Embodiment 174. The use of embodiments 171 or 172, wherein the therapeutically effective dose is administered one time a day.

Embodiment 175. The use of any one of embodiments 171-174, wherein the therapeutically effective dose is administered daily on a continuous 28-day cycle.

Embodiment 176. The use of any one of embodiments 171-174, wherein the therapeutically effective dose is administered daily on a continuous 21-day cycle.

Embodiment 177. The use of any one of embodiments 171-176, wherein Compound A is used as a hydrate or a monohydrate form thereof.

Embodiment 178. The use of any one of embodiments 171-177, wherein said disorder or condition is a cancer selected from lymphomas, leukemias, carcinomas, and sarcomas, e.g. non-Hodgkin's lymphoma (NHL (including B-cell NHL)), diffuse large B-cell lymphoma (DLBCL), mantle cell lymphoma (MCL), follicular lymphoma (FL), mucosa-associated lymphoid tissue (MALT) lymphoma, marginal zone lymphoma, T-cell lymphoma, Hodgkin's lymphoma, Burkitt's lymphoma, multiple myeloma, chronic lymphocytic leukemia (CLL), small lymphocytic lymphoma (SLL), Waldenström macroglobulinemia, lymphoblastic T cell leukemia, chronic myelogenous leukemia (CML), hairy-cell leukemia, acute lymphoblastic T cell leukemia, plasmacytoma, immunoblastic large cell leukemia, megakaryoblastic leukemia, acute megakaryocyte leukemia, promyelocytic leukemia, erythroleukemia, brain (gliomas), glioblastomas, breast cancer, colorectal/colon cancer, prostate cancer, lung cancer including non-small-cell, gastric cancer, endometrial cancer, melanoma, pancreatic cancer, liver cancer, kidney cancer, squamous cell carcinoma, ovarian cancer, sarcoma, osteosarcoma, thyroid cancer, bladder cancer, head & neck cancer, testicular cancer, Ewing's sarcoma, rhabdomyosarcoma, medulloblastoma, neuroblastoma, cervical cancer, renal cancer, urothelial cancer, vulval cancer, esophageal cancer, salivary gland cancer, nasopharyngeal cancer, buccal cancer, cancer of the mouth, primary and secondary central nervous system lymphoma, transformed follicular lymphoma, diseases/cancer caused by API2-MALT1 fusion, and GIST (gastrointestinal stromal tumor).

Embodiment 179. The use of any one of embodiments 171-177, wherein said disorder or condition is an immunological disease selected from autoimmune and inflammatory disorders, e.g. arthritis, rheumatoid arthritis (RA), psoriatic arthritis (PsA), inflammatory bowel disease, gastritis, ankylosing spondylitis, ulcerative colitis, pancreatitis, Crohn's disease, celiac disease, multiple sclerosis, systemic lupus erythematosus, lupus nephritis, rheumatic fever, gout, organ or transplant rejection, chronic allograft rejection, acute or chronic graft-versus-host disease, dermatitis including atopic, dermatomyositis, psoriasis, Behcet's diseases, uveitis, myasthenia gravis, Grave's disease, Hashimoto thyroiditis, Sjoergen's syndrome, blistering disorders, antibody-mediated vasculitis syndromes, immune-complex vasculitides, allergic disorders, asthma, bronchitis, chronic obstructive pulmonary disease (COPD), cystic fibrosis, pneumonia, pulmonary diseases including oedema, embolism, fibrosis, sarcoidosis, hypertension and emphysema, silicosis, respiratory failure, acute respiratory distress syndrome, BENTA disease, berylliosis, and polymyositis.

Embodiment 180. The use of any one of embodiments 171-179, wherein the use comprises: an amount sufficient to maintain a plasma level of Compound A from about 2,300 ng/mL to about 9,300 ng/ml; an amount sufficient to maintain a plasma level of Compound A from about 2,320 ng/ml to about 9,280 ng/ML; an amount sufficient to maintain a plasma level of Compound A from about 3,000 ng/ml to about 9,000 ng/ml; an amount sufficient to maintain a plasma level of Compound A from about 3,500 ng/ml to about 8,500 ng/ml; an amount sufficient to maintain a plasma level of Compound A from about 4,000 ng/mL to about 8,000 ng/ml; an amount sufficient to maintain a plasma level of Compound A from about 4,000 ng/mL to about 6,000 ng/ml; an amount sufficient to maintain a plasma level of Compound A of at least 4,600 ng/ml; an amount sufficient to maintain a plasma level of Compound A from about 4,500 ng/ml to about 4,750 ng/ml; an amount sufficient to maintain a plasma level of Compound A from about 4,640 ng/ml; an amount sufficient to maintain a plasma level of Compound A from about 4,550 to about 4,700 ng/ml; or an amount sufficient to maintain a plasma level of Compound A from about 4,550 to about 4,680 ng/ml.

Embodiment 181. The use of any one of embodiments 171-180, wherein the use comprises a pharmaceutical composition of Compound A or a solvate or pharmaceutically acceptable salt form thereof further comprising a pharmaceutically acceptable carrier, a pharmaceutically acceptable excipient, and/or a pharmaceutically acceptable diluent.

Embodiment 182. The use of any one of embodiments 171-181, wherein said disorder or condition is relapsed or refractory to prior treatment.

Embodiment 183. The use of any one of embodiments 171-181, wherein said disorder or condition is relapsed or refractory to prior treatment with a Bruton tyrosine kinase inhibitor (BTKi).

Embodiment 184. The use of 1-(1-oxo-1,2-dihydroisoquinolin-5-yl)-5-(trifluoromethyl)-N-[2-(trifluoromethyl)pyridin-4-yl]-1H-pyrazole-4-carboxamide (Compound A):

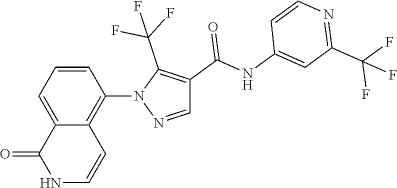 [SMILES: O=C(Nc1ccnc(C(F)(F)F)c1)c1cnn(-c2cccc3c(=O)[nH]ccc23)c1C(F)(F)F]

or a pharmaceutically acceptable salt form thereof for the manufacture of a medicament for the treatment of a method of reducing the T_{reg}/T_{eff} ratio in a patient suffering from a disorder or condition that is affected by the inhibition of MALT1 comprising administering a therapeutically effective dose of Compound A or a pharmaceutically acceptable salt form thereof to said patient.

Embodiment 185. The use of embodiment 184, wherein the therapeutically effective dose is:
from about 50 to about 500 mg;
from about 100 to about 500 mg; or
from about 100 to about 400 mg.

Embodiment 186. The use of embodiment 184, wherein the therapeutically effective dose is:
from about 150 to about 350 mg;
from about 200 to about 350 mg;
from about 275 to about 375 mg; or about 300 mg.

Embodiment 187. The use of any one of embodiments 184-186, wherein the therapeutically effective dose is divided in half, said half dose being administered twice (two times) a day.

Embodiment 188. The use of any one of embodiments 184-186, wherein the therapeutically effective dose is administered one time a day.

Embodiment 189. The use of any one of embodiments 184-188, wherein the therapeutically effective dose is administered daily on a continuous 28-day cycle.

Embodiment 190. The use of any one of embodiments 184-188, wherein the therapeutically effective dose is administered daily on a continuous 7-day to 21-day cycle.

Embodiment 191. The use of embodiments 189 or 190, wherein the cycle is repeated.

Embodiment 192. The use of any one of embodiments 184-191, wherein the method further comprises determining the proportion of CD8⁺ T_{eff} and CD4⁺CD25^{hi}FOXP3^{hi} T_{reg} cells.

All embodiments described herein for methods of treating a disorder or condition, are also applicable for use in treating said disorder or condition.

All embodiments described herein for methods of treating a disorder or condition, are also applicable for use in a method of treating a disorder or condition.

While preferred embodiments of the present invention have been shown and described herein, it will be apparent to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention and that embodiments within the scope of these claims and their equivalents be covered thereby

1-74. (canceled)

75. A method of treating cancer in a subject comprising administering a therapeutically effective dose ranging from about 50 mg to about 1000 mg of 1-(1-oxo-1,2-dihydroisoquinolin-5-yl)-5-(trifluoromethyl)-N-[2-(trifluoromethyl)pyridin-4-yl]-1H-pyrazole-4-carboxamide (Compound A): 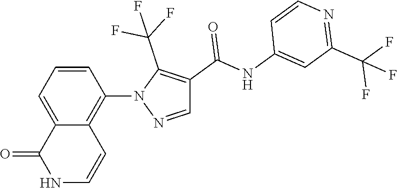 [SMILES: O=C(Nc1ccnc(C(F)(F)F)c1)c1cnn(-c2cccc3c(=O)[nH]ccc23)c1C(F)(F)F]
or a pharmaceutically acceptable salt form thereof to said subject.

76. The method of claim 75, wherein the therapeutically effective dose is from about 50 to about 500 mg.

77. The method of claim 75, wherein the therapeutically effective dose is from about 100 to about 400 mg.

78. The method of claim 75, wherein the therapeutically effective dose is from about 150 to about 300 mg.

79. The method of claim 75, wherein the therapeutically effective dose is administered one time a day.

80. The method of claim 75, where in the cancer is selected from non-Hodgkin's lymphoma (NHL), diffuse large B-cell lymphoma (DLBCL), mantle cell lymphoma (MCL), follicular lymphoma (FL), mucosa-associated lymphoid tissue (MALT) lymphoma, marginal zone lymphoma, T-cell lymphoma, Hodgkin's lymphoma, Burkitt's lymphoma, multiple myeloma, chronic lymphocytic leukemia (CLL), small lymphocytic lymphoma (SLL), Waldenström macroglobulinemia, lymphoblastic T cell leukemia, chronic myelogenous leukemia (CML), hairy-cell leukemia, acute lymphoblastic T cell leukemia, plasmacytoma, immunoblastic large cell leukemia, megakaryoblastic leukemia, acute megakaryocyte leukemia, promyelocytic leukemia, erythroleukemia, glioblastomas, breast cancer, colon cancer, prostate cancer, lung cancer, gastric cancer, endometrial cancer, melanoma, pancreatic cancer, liver cancer, kidney cancer, squamous cell carcinoma, ovarian cancer, sarcoma, osteosarcoma, thyroid cancer, bladder cancer, head & neck cancer, testicular cancer, Ewing's sarcoma, rhabdomyosarcoma, medulloblastoma, neuroblastoma, cervical cancer, renal cancer, urothelial cancer, vulval cancer, esophageal cancer, salivary gland cancer, nasopharyngeal cancer, buccal cancer, cancer of the mouth, primary and secondary central nervous system lymphoma, transformed follicular lymphoma, cancer caused by API2-MALT1 fusion, and GIST (gastrointestinal stromal tumor).

81. The method of claim 80, wherein the cancer is selected from non-Hodgkin's lymphoma (NHL), diffuse large B-cell lymphoma (DLBCL), marginal zone lymphoma, mantle cell lymphoma (MCL), follicular lymphoma (FL), transformed follicular lymphoma, chronic lymphocytic leukemia, and Waldenström macroglobulinemia.

82. The method of claim 75, wherein said subject is relapsed or refractory to prior treatment with a Bruton tyrosine kinase inhibitor (BTKi).

83. The method of claim 75, wherein Compound A is used as a hydrate or a monohydrate form thereof.

84. A method of treating cancer in a subject comprising administering to the subject a therapeutically effective dose of about 100 mg to about 300 mg of Compound A.

85. The method of claim 84, wherein the cancer is selected from non-Hodgkin's lymphoma (NHL), diffuse large B-cell lymphoma (DLBCL), marginal zone lymphoma, mantle cell lymphoma (MCL), follicular lymphoma (FL), transformed follicular lymphoma, chronic lymphocytic leukemia, and Waldenström macroglobulinemia.

86. The method of claim 84, wherein the method comprises administering about 100 mg to about 300 mg of Compound A once daily for 7 to 21 continuous day cycle, and optionally 3-10 cycles.

87. The method of claim 84, wherein the method comprises administering a therapeutically effective dose 100 mg to about 300 mg of Compound A twice daily for 7 days followed by administration of about 100 mg to about 300 mg of Compound A once a day until remission.

88. The method of claim 84, wherein said subject is relapsed or refractory to prior treatment with a Bruton tyrosine kinase inhibitor (BTKi).

89. The method of claim 84, wherein Compound A is used as a hydrate or a monohydrate form thereof.

90. A method of treating cancer in a subject comprising administering a therapeutic effective dose of 1-(1-oxo-1,2-dihydroisoquinolin-5-yl)-5-(trifluoromethyl)-N-[2-(trifluoromethyl)pyridin-4-yl]-1H-pyrazole-4-carboxamide (Compound A): 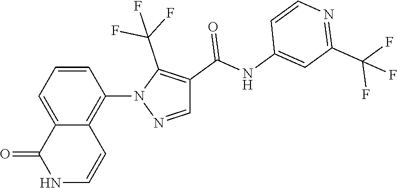 [SMILES: O=C(Nc1ccnc(C(F)(F)F)c1)c1cnn(-c2cccc3c(=O)[nH]ccc23)c1C(F)(F)F]
or a pharmaceutically acceptable salt form thereof to said subject in an amount sufficient to maintain a plasma level of Compound A from about 2 mg/ml to about 120 mg/ml, about 2 mg/ml to about 100 mg/ml, about 2 mg/ml to about 80 mg/ml, about 2 mg/ml to about 60 mg/ml, or about 2 mg/ml to about 20 mg/ml.

91. A method of treating cancer in a subject in need of treatment comprising administering a therapeutic effective dose of 1-(1-oxo-1,2-dihydroisoquinolin-5-yl)-5-(trifluoromethyl)-N-[2-(trifluoromethyl)pyridin-4-yl]-1H-pyrazole-4-carboxamide (Compound A): 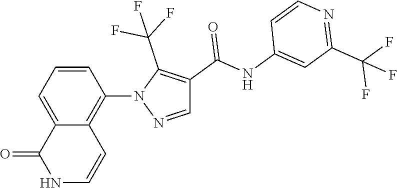 [SMILES: O=C(Nc1ccnc(C(F)(F)F)c1)c1cnn(-c2cccc3c(=O)[nH]ccc23)c1C(F)(F)F]
or a pharmaceutically acceptable salt form thereof to said subject in an amount sufficient to achieve an AUC from about 50 mg·h/ml to about 2500 mg·h/ml, about 50 mg·h/ml to about 2000 mg·h/ml, about 50 mg·h/ml to about 1500 mg·h/ml, about 50 mg·h/ml to about 1000 mg·h/ml, or about 50 mg·h/ml to about 600 mg·h/ml.
